(19) United States
(12) Patent Application Publication
(10) Pub. No.: US 2023/0060588 A1
(43) Pub. Date: Mar. 2, 2023

(54) METHODS OF AMELIORATING AND/OR PREVENTING CORONAVIRUS-RELATED INFECTIONS

(71) Applicant: Glycure LLC, Aurora, CO (US)

(72) Inventors: Xi CHEN, Aurora, CO (US); Jian ZHANG, Aurora, CO (US)

(73) Assignee: Glycure LLC, Aurora, CO (US)

(21) Appl. No.: 17/898,653

(22) Filed: Aug. 30, 2022

Related U.S. Application Data
(60) Provisional application No. 63/239,093, filed on Aug. 31, 2021.

(51) Int. Cl. A61K 31/7012; A61K 31/7004; A61K 31/7008; A61P 31/14

(52) U.S. Cl. CPC A61K 31/7012; A61K 31/7004; A61K 31/7008; A61P 31/14

(57) ABSTRACT

The invention provides a method of ameliorating and/or preventing a coronavirus infection in a subject, the method comprising administering to the subject a therapeutically effective amount of a sugar or a derivative thereof. The invention also provides a method of ameliorating and/or preventing a coronavirus infection in a subject, the method comprising upregulating sialylation of a glycan, downregulating N-glycosylation, and/or downregulating O-glycosylation of a virus particle of the coronavirus.

### CROSS-REFERENCE TO RELATED APPLICATIONS

This patent application claims the priority of copending U.S. Provisional Application 63/239,093, filed Aug. 31, 2021, which is hereby incorporated by reference in its entirety.

### BACKGROUND OF THE INVENTION

Coronavirus Disease 2019 (COVID-19), an infectious disease resulting from the Severe Acute Respiratory Syndrome Coronavirus 2 (SARS-CoV-2) infection, has ravaged the world since December of 2019. Throughout history, there were several outbreaks of coronavirus, including the Severe Acute Respiratory Syndrome (SARS) during 2002-2003 in South East Asia and Middle East Respiratory Syndrome (MERS) in 2012 in the Middle East and 2015 in Korea (Cui et al., Nat. Rev. Microbiol., 17: 181-192 (2019)). However, none of these outbreaks caused such a huge impact on public heath as COVID-19. The World Health Organization (WHO) officially declared COVID-19 as a global pandemic on Mar. 11, 2020 due to the rapid dissemination of SARS-CoV-2. By July 2021, there were over 188 million confirmed cases and 4 million deaths globally.

SARS-CoV-2 is a member of the Coronavirinae subfamily which belongs to the family Coronaviridae and the order Nidovirales. Based on genetic and phylogenetic features, Coronavirinae subfamily is further classified into four genera: α-, β-, γ-, and δ-CoV (Cui et al., Nat. Rev. Microbiol, 17: 181-192 (2019)). SARS-CoV-2 is a novel betacoronavirus, where the genome organization is shared with other betacoronaviruses. For example, SARS-CoV-2 shares 50% genome sequence identity with MERS-CoV and 79% genome sequence identity with SARS-CoV (Lu et al., Lancet, 395: 565-574 (2020)). The genome sequence of SARS-CoV-2 contains several functional open reading frames (ORFs) that are arranged in order from 5′ to 3′: non-structural proteins (ORF1a/ORF1b), spike (S), envelope (E), membrane (M) and nucleocapsid (N). In addition, several ORFs are interspersed between those structural genes. These putative ORFs encode accessory proteins critical for transcription and virus replication (Chan et al., Emerg. Microbes Infect., 9: 221-236 (2020). ORF1a and ORF1b encode two coterminal polyproteins pp1a and pp1b, which are proteolytically cleaved into 16 non-structural proteins (NSP1-16), including viral protease, RNA dependent RNA polymerase (RdRp), and other viral accessory proteins, which are all critical for SARS-CoV-2 replication (Cui et al., Nat. Rev. Microbiol, 17: 181-192 (2019)).

Coronavirus enters the host cells by either host receptor-mediated fusion or fusing after being swallowed into an endosome. For example, the spike (S) protein of SARS-CoV-2 plays a key role in the receptor recognition and cell membrane fusion process. The spike (S) protein is composed of two subunits, S1 and S2. The S1 subunit contains a receptor-binding domain (RBD) that recognizes and binds to the host receptor, while the S2 subunit mediates viral cell membrane fusion (Huang et al., Acta Pharmacol. Sin., 41: 1141-1149 (2020)). For example, SARS-CoV-2 viruses utilize the human angiotensin-converting enzyme 2 (ACE2) type I membrane protein as an entry receptor (Hoffmann et al., Cell, 181: 271-280 (2020)). Upon binding to the ACE2 receptor by viral S protein, with the help of transmembrane serine protease 2 (TMPRSS2) on the cell surface, SARS-Cov-2 enters the host cells through direct membrane fusion to the host cells. Alternatively, SARS-CoV-2 can also enter the host cells by clathrin-mediated endocytosis. In this case, after binding to the ACE2 receptor by viral S protein, endosomal proteases prime the viral particle for viral—endosomal membrane fusion to swallow viruses into endosomes (Wilde et al., Curr. Top. Microbiol. Immunol., 419: 1-42 (2018)). In addition to the ACE2 receptor, CD147 can also serve as an alternative receptor for viral entry by endocytosis route (Wang et al., Signal Transduct. Target. Ther., 5: 1-10 (2020)). Neuropilin-1 (NRP1) and lectins are other host factors that facilitate SARS-CoV-2 entry and infectivity (Lempp et al., Nature, 598: 342-347 (2021); Cantuti-Castelvetri et al., Science, 370: 856-860 (2020); Daly et al., Science, 370: 861-865 (2020)).

After entering host cells, the SARS-CoV-2 genome is released into the cytoplasm and viral replication is initiated. Viral RNA is translated into long strings of amino acids by ribosomes, which are then cleaved into 16 non-structural proteins (NSPs) critical for viral RNA synthesis. Viral RNAs are translated into 26 known viral proteins which are important for virion assembly (V'kovski et al., Nat. Rev. Microbiol., 193(19): 155-170 (2020)). New SARS-CoV-2 particles are assembled in the ER-Golgi intermediate compartment (ERGIC) followed by spike protein glycosylation and maturation in the Golgi apparatus (Hoffmann et al., Cell, 181: 271-280 (2020)).

Generating progeny virions is highly dependent on the host metabolism. Viruses exploit the metabolic machinery of host cells to efficiently biosynthesize building blocks for optimal replication capacity. SARS-CoV-2 hijacks multiple key metabolic pathways of host cells for its reproduction advantage. In one study, by using in vitro infection assays of lung epithelial cells (Calu-3), the researchers report that glycolysis and glutaminolysis are essential for SARS-CoV-2 replication (Krishnan et al., bioRxiv (2021), 48 pp.), and pharmacologically blocking these metabolic pathways significantly reduced virus production. In another study, by using in vitro infection assays of Vero-E6 expressing TMPRSS2 (Vero-E6 TMPRSS2+) and A549 cells that overexpress ACE2 (A549 ACE2+), the researchers demonstrate that SARS-CoV-2 induces glycolysis and remodels host folate and one-carbon metabolism at the post-transcriptional level to support optimal viral replication (Zhang et al., Nat. Commun., 12: 1676-1686 (2021)). The study shows that SARS-CoV-2 replication is reduced by methotrexate, an inhibitor notably targets folate and one-carbon metabolism. In another study, the researchers report that elevated glucose levels and glycolysis promote SARS-CoV-2 replication and cytokine production in human monocytes, which in-turn induces T cell dysfunction and epithelial cell death, thereby promoting host pathogenesis (Codo et al., Cell Metab., 32: 437-446 (2020)). Specifically, SARS-CoV-2 infection triggers the production of ROS in mitochondria, which stabilizes hypoxia-inducible factor-1α (HIF-1α) and consequently promotes glycolysis. As a result, metabolism shift of monocytes inhibits T cell response and enhances epithelial cell death. In another study, by metabolic profiling changes conferred by SARS-CoV-2 infection in kidney epithelial cells (Vero cells and HEK293T cells that overexpress ACE2) and lung air-liquid interface cultures, the researchers found that SARS-CoV-2 infection enhances glucose carbon entry into the TCA cycle through increased pyruvate carboxylase expression. At the same time, the viral infection also reduces oxidative glutamine metabolism while maintaining reductive carboxylation. Consistent with these metabolism changes, SARS-CoV-2 infection increases the activity of mTORC1 (Mullen et al., Nat. Commun., 12: 1876 (2021)). In another study, researchers analyzed transcriptomic data obtained from different human respiratory cell lines and patient samples and revealed that SARS-CoV-2 infection dysregulated host glycolysis, mitochondrial, amino acid, glutathione, lipid metabolism, and polyamine synthesis (Moolamalla et al., bioRxiv (2020), 30 pp.). The studies show that carbohydrate and amino acid metabolism, polyamine synthesis, and redox homeostasis are major metabolic pathways hijacked by SARS-CoV-2. In another study, the switch of host lipid metabolism has also been speculated to be critical for optimal SARS-CoV-2 replication (Casari et al., Prog. Lipid Res., 82: 101092 (2021)). These studies show that SARS-CoV-2 utilizes host metabolic pathways for optimal propagation.

As one of the paths to enter the host cells, the spike (S) protein of SARS-CoV-2 mediates cell entry and membrane fusion by recognizing the host ACE2 receptor. Both viral S protein and host ACE2 receptor are highly glycosylated, including sites near their binding interface. The SARS-CoV-2 S gene encodes 22 N-linked glycan sequons (Watanabe et al., Science, 369: 330-333 (2020)) and at least 17 O-glycosites (Tian et al., Cell Res., 31: 1123-1125 (2021); Bagdonaite et al., Viruses, 13: 551 (2021)). Experimentally, by using recombinant S proteins expressed from HEK293 cells, researchers identified that most N-glycosylation sequons are occupied by oligomannose, hybrid, complex, sialylated, and fucosylated structures (Watanabe et al., Science, 369: 330-333 (2020); Zhang, Y. et al., Mol. Cell. Proteomics, 20, 100058 (2021); Shajahan et al., Glycobiology, 30(12): 981-988 (2020)). The distribution of these structures is highly dependent on the protein structure and host expression system (Watanabe et al., Science, 369: 330-333 (2020); Shajahan et al., Glycobiology, 30(12): 981-988 (2020)). O-linked glycans have also been observed experimentally. One study identified two O-glycosylation sites at the receptor-binding domain (RBD) of subunit S1 (Shajahan et al., Glycobiology, 30(12): 981-988 (2020)). The other study identified a novel glycopeptide near the furin cleavage site of the S protein (Sanda et al., Anal. Chem., 93: 2003-2009 (2021)). Another study identified 17 sites of O-glycosylation on the S protein extracted from SARS-CoV-2 virions (Tian et al., Cell Res., 31: 1123-1125 (2021)). These studies show that glycans play essential roles in modulating the conformational dynamics of S protein. In that regard, deletion of N-glycans through mutation of asparagine in certain sequons of S protein reduces its binding to ACE2 (Casalino, et al., ACS Cent. Sci., 6: 1722-1734 (2020)). On the other hand, the host ACE2 gene encodes 7 N-linked glycan sequons. Glycans on the ACE2 receptor contribute substantially to the binding of S protein. By using atomistic molecular dynamics simulation, one study found that N90 glycan of ACE2 weakens the binding of SARS-CoV-2 S protein while N322 glycan strengthens such binding (Mehdipour et al., PNAS, 118(19): 1-8 (2021)). Thus, glycans play critical roles in modulating SARS-CoV-2 S protein—host ACE2 receptor interaction.

Sialic acids are nine-carbon acidic α-keto sugars located at the end of glycans (e.g., N-glycans or O-glycans). N-linked glycosylation sites of both viral S protein and host ACE2 receptor are predominantly modified by hybrid or complex N-glycans (Zhao et al., bioRxiv, 30: 1133-1134 (2020); Watanabe et al., Science, 369: 330-333 (2020)). At the terminus of these N-glycans, sialic acid can be additionally added. Similarly, all of the identified O-glycans can be terminated with sialic acid. Sialoglycan microarrays were initially used to evaluate S protein binding; however, it didn't detect significant signals (Hao et al., bioRxiv, (2020), 18 pp.). The result needs to be further verified as immobilized sialoglycans may not fully mimic native glycoconjugates floating in the flexible plasma membrane. Another study found that recombinant S-protein binds to a single sialic acid, to a less extent, α2,3-sialyllactose and α2,6-sialyllactose present on the polymer-stabilized gold nanoparticles (Baker et al., ACS Cent. Sci., 6: 2046-2052 (2020)). This result deserves further investigation as the assay is irrelevant to the native biological condition. In-vitro and ex-vivo studies were further carried out to reveal the effect of sialic acids on host cell—viral particle interaction. In one study, neuraminidase (Arthrobacter ureafaciens) treatment of HEK293T ACE2+ cells enhanced recombinant RBD and 51 binding by 26% and 56%, respectively (Yang et al., elife, 9: 1-44 (2020)). In another study, neuraminidase (Arthrobacter ureafaciens) treatment of Calu-3 human airway cells increased SARS-CoV infection by 492% and SARS-CoV-2 infection by 80.3% (quantified at 24 hours post-infection) (Chu et al., Nat. Commun., 12: 134 (2021)). By conducting ACE2 glycosylation mutant study, the same study also found that sialic acid on ACE2 prevents SARS-CoV-2 S protein and ACE2 binding (Chu et al., Nat. Commun., 12: 134 (2021)). In another study, researchers generated a panel of ACE2 variants with modified glycans. By analyzing binding between SARS-CoV-2 S protein and each of these ACE2 variants, they observed a modest decrease in binding affinity if ACE2 glycans are hypersialylated; upon removing sialic acids, a statistically significant increase in binding affinity was observed (Allen et al., J. Mol. Biol., 433: 166762 (2021)).

Thus, previous studies focused on the sialic acids present on the ACE2 receptor or other glycoconjugates on the host cell surface. However, it is still unknown whether sialic acids on the viral S protein prevent SARS-CoV-2 S protein—ACE2 interaction.

The SARS-CoV-2 S protein is heavily glycosylated. There are 22 N-glycosites linked with various types of N-glycans, which includes the high-mannose type of N-glycans, the hybrid type of N-glycans, and the complex type of N-glycans (Tian et al., Cell Res., 31: 1123-1125 (2021)). Dynamic simulations of SARS-CoV-2 S protein suggest a critical role of N-glycans in stabilizing the protein (Henderson et al., Nat. Struct. Mol. Biol., 27: 925-933 (2020); Choi et al., J. Chem. Theory and Comput., 17: 2479-2487 (2021)). Beyond the molecular dynamics simulations, one study found that deletion of N-glycans through mutation of asparagine in certain sequons of S protein reduces its binding to ACE2 (Casalino et al., ACS Cent. Sci., 6: 1722-1734 (2020)). Another study found that, in the host cells, blocking N-glycan biosynthesis of SARS-CoV-2 S protein partially reduced ACE2 binding (Yang et al., elife, 9: 1-44 (2020)). In this study, researchers generated [N]⁻293 T cells, in which the N-glycan branching β1,2GlcNAc-transferase MGAT1 is knocked out. By expressing SARS-CoV-2 S protein in [N]⁻293 T cells, researchers obtained SARS-CoV-2 S protein without the complex type of N-glycans. They found that SARS-CoV-2 S protein without complex N-glycans showed reduced binding to ACE2. Accordingly, pseudotyped virus generated from [N]⁻293 T cells showed significantly less infection. Consistent with these findings, another study showed that inhibition of N-glycan biosynthesis in the host cells blocks SARS-CoV-2 infection (Casas-Sanchez et al., mBio, 13(1): e03718-21 (2022)). In this study, PNGase F was used to remove N-glycans on the SARS-CoV-2 viral particles. PNGase F is an amidase that removes all types of N-glycans except those with core α1,3-fucose. SARS-CoV-2 viral particles treated with PNGase F before inoculation showed significantly less infection to the host cells. N-glycans on the RBD are essential for SARS-CoV-2 infection. These N-glycans (N331 and N343) are thought to support the opening conformation of active RBD (Sztain et al., Nat. Chem., 13: 963-968 (2021)) and deletion of these N-glycans drastically reduced SARS-CoV-2 infectivity (Li et al., Cell, 182: 1284-1294 (2020)). Together, these studies suggest that reduction of N-glycans play an important role in SARS-CoV-2 infectivity.

There are multiple O-glycosites on the SARS-CoV-2 S protein. A total of 25 O-glycosites were identified on the SARS-CoV-2 S proteins expressed in the HEK293F cells (Bagdonaite et al., Viruses, 13: 551 (2021)) and a total of 17 O-glycosites were identified on the same proteins directly extracted from SARS-CoV-2 virions (Tian et al., Cell Res., 31: 1123-1125 (2021)). One study found that, in host cells, blocking O-glycan extension of SARS-CoV-2 S protein moderately enhanced ACE2 binding; however, significantly reduced viral infection (Yang et al., elife, 9: 1-44 (2020)). In this study, researchers generated [O]⁻293T cells, in which the core-1 O-glycan forming galactosyltransferase C1GalT1 is knocked out to block the extension of core 1 and core 2 structures. By expressing SARS-CoV-2 S protein in [O]⁻293 T cells, researchers obtained SARS-CoV-2 S protein without core 1 and core 2 glycan structures. Although SARS-CoV-2 S protein without these core structures showed moderately enhanced binding to ACE2, pseudotyped virus generated from the same [O]⁻293 T cells showed significantly less infection. Together, these studies suggest that reduction of O-glycans play an important role in SARS-CoV-2 infectivity.

SARS-CoV-2 infections evoke cytokine storm that highly correlates with mortality (Ragab et al., Front. Immunol. 11: 1446 (2020)). Cytokine storm leads to acute respiratory distress syndrome (ARDS), which is characterized by lung inflammation, neutrophil infiltration and increased oxidative stress. ARDS also accompanies excessive production of plasma pro-inflammatory cytokines IL-2, 7, 10, and 17; GM-CSF; interferon-γ-inducible protein 10; MCP-1; macrophage inflammatory protein-1 alpha; and TNF-α (Mehta et al., Lancet, 395: 1033-1034 (2020)). Systematic inflammation causes tissue damage and multi-organ failure. Cytokine storm appears to be one of the common causes of mortality.

Consequently, prevention and treatment of SARS-CoV-2 infections can include strategies that target virus entry, virus replication, release of virus and host-directed metabolism. Additionally, methods to manage the COVID-19 cytokine storm can improve survival rates and reduce mortality. Currently, only a few drugs have been conditionally approved with only modest efficacies. These drugs target virus entry, virus replication, or control the cytokine storm. While approved vaccines and monoclonal antibodies are thought to be the long-term solution for COVID-19, these therapies have already faced challenges as the outbreak continues precariously. Some vaccines and antibody-based therapies become less effective as SARS-CoV-2 strains with escape mutations continuously evolve. These therapies are strain-specific and require development time for each new strain. (Harvey et al., Nat. Rev. Microbiol. 19: 409-424 (2021); Wang et al., Genomics, 113: 2158-2170 (2021); Starr et al., Science, 371: 850-854 (2021); Wibmer et al., Nat. Med., 27: 622-625 (2021); Wang et al., Nature, 593: 130-135 (2021)).

Thus, treatment options for ameliorating and/or preventing the severity of SARS-CoV-2 infections is still urgently needed. The present invention provides such methods of ameliorating and/or preventing a coronavirus infection. These and other advantages of the invention, as well as additional inventive features, will be apparent from the description of the invention provided herein.

### BRIEF SUMMARY OF THE INVENTION

The present invention provides a method of ameliorating and/or preventing a coronavirus infection (e.g., a SARS-CoV-2 infection) in a subject, the method comprising administering to the subject a therapeutically effective amount of a sugar or a derivative thereof selected from D-mannose, fructose, neuraminic acid, mannosamine, glucosamine, galactosamine, a metabolite thereof, a prodrug thereof, or a combination thereof.

The present invention also provides a method of ameliorating and/or preventing a coronavirus infection (e.g., a SARS-CoV-2 infection) in a subject, the method comprising upregulating sialylation of a glycan, downregulating N-glycosylation, and/or downregulating O-glycosylation of a virus particle of the coronavirus.

### BRIEF DESCRIPTION OF THE DRAWINGS

FIG. 1 shows the effect of D-mannose on ACE2 expression for both D-mannose treated (“Pre”) and non-treated cells (“Control”), as analyzed by flow cytometry and summarized with a bar graph, as described in Example 1. Graph Abbreviations: Control=A549 ACE2+ cells without treatment; Pre=A549 ACE2+ cells treated with D-mannose until addition of virions; None=no anti-ACE2 antibody was added; ACE2 AB=anti-ACE2-antibody was added.

FIG. 2 is a bar graph showing the effect of D-mannose on SARS-CoV-2 replication for (i) non-treated cells (“Control”), (ii) D-mannose pretreated cells (“Pre”), and (iii) D-mannose continuously treated cells (“Pre/Cont.”), as analyzed by a Celigo image cytometer, as described in Example 1. * P≤0.05, ** P≤0.01, ***P≤0.001, ****P≤0.0001. Graph Abbreviations: Control=A549 ACE2+ cells without treatment; Pre=A549 ACE2+ cells treated with D-mannose until addition of virions; Pre/Cont.=A549 ACE2+ cells treated with D-mannose and continued with the same treatment until the end of the experiment; MOI=multiplicity of infection.

FIG. 3 is a bar graph showing the effect of D-mannose on total cell count for (i) non-treated cells (“Control”), (ii) D-mannose pretreated cells (“Pre”), and (iii) D-mannose continuously treated cells (“Pre/Cont.”), as analyzed by a Celigo image cytometer, as described in Example 1. Graph Abbreviations: Control=A549 ACE2+ cells without treatment; Pre=A549 ACE2+ cells treated with D-mannose until addition of virions; Pre/Cont.=A549 ACE2+ cells treated with D-mannose and continued with the same treatment until the end of the experiment.

FIG. 4 is a table (Table. 1) showing a list of glycan structures on the Catch-All microarray as described in Example 2. Abbreviations: Glc=glucose; Man=mannose; Gal=galactose; GlcNAc=N-Acetyl-D-Glucosamine; GalNAc=N-Acetyl-D-Galactosamine; Fuc=fucose; Neu5Ac=N-Acetyl-Neuraminic Acid; Neu5Gc=N-Glycolyl-Neuraminic acid.

FIG. 5 is a table (Table 2) showing a list of glycan structures on the O-glycan microarray (from ZBiotech) as described in Example 2. Graph Abbreviations: Glc=glucose; Man=mannose; Gal=galactose; GlcNAc=N-Acetyl-D-Glucosamine; GalNAc=N-Acetyl-D-Galactosamine; Fuc=fucose; Neu5Ac=N-Acetyl-Neuraminic Acid; Neu5Gc=N-Glycolyl-Neuraminic acid.

FIGS. 6A-6C provide bar graphs showing the binding specificities of lectins that preferentially recognize O-glycosylation. The binding specificities of Peanut Agglutinin (PNA) lectin (see FIG. 6A) and Vicia Villosa (VVL) lectin (see FIG. 6C) were determined by Catch-All glycan microarray. The binding specificity of PNA (see FIG. 6B) was additionally validated by O-glycan microarray (from ZBiotech) as descried in Example 2. Graph Abbreviation: PC1=Biotin-PEG-amine (BIO-PEG-NH2).

FIGS. 7A-7D provide bar graphs showing the binding specificities of lectins that preferentially recognize N-glycosylation. The binding specificities of Calystegia Sepium (Calsepa) lectin (see FIG. 7A), Erythrina Cristagalli (ECL) lectin (see FIG. 7B), and Musa Paradisiaca (BanLec) lectin (see FIG. 7D) were determined by Catch-All glycan microarray. The binding specificity of ECL (see FIG. 7C) was additionally validated by O-glycan microarray (from ZBiotech) as descried in Example 2. Graph Abbreviation: PC1=Biotin-PEG-amine (BIO-PEG-NH2).

FIGS. 8A and 8B provide bar graphs showing the binding specificities of lectins that recognize fucose of N- or O-linked glycans. The binding specificities of Ralstonia solanacearum (RSL) lectin (see FIG. 8A) and Pisum Sativum Agglutinin (PSA) lectin (see FIG. B) were determined by Catch-All glycan microarray as descried in Example 2. Graph Abbreviation: PC1=Biotin-PEG-amine (BIO-PEG-NH2).

FIGS. 9A-9D provide bar graphs showing the binding specificities of lectins that recognize terminal sialic acids on N- or O-linked glycans. The binding specificities of Sambucus Nigra (SNA) lectin (see FIG. 9A), Maackia Amurensis Lectin I (MAL1) (see FIG. 9B), and Maackia Amurensis Lectin II (MAL2) (see FIG. 9C) were determined by Catch-All glycan microarray. The binding specificity of MAL2 (see FIG. 9D) was additionally validated by O-glycan microarray (from ZBiotech) as descried in Example 2. Graph Abbreviation: PC1=Biotin-PEG-amine (BIO-PEG-NH2).

FIG. 10 provides bar graphs showing the effect on hACE2 binding, ECL binding, and SNA binding, exhibited by SARS-CoV-2 S trimers, as a result of modulating the prevalence of sialic acids on the S trimers, as described in Example 3. * P≤0.05, ** P≤0.01, ***P≤0.001, ****P≤0.0001. Graph Abbreviations: Vehicle=vehicle buffer only; Neu=neuraminidase from Clostridium perfringens; SiaT=sialyltransferase; α(2,3) or α(2,6)SiaT=α2,3 or α2,6-sialyltransferase.

FIG. 11 provides images of stained array slides showing the level of neuraminidase on SARS-CoV-2 S trimers before ACE2 addition, as described in Example 3.

FIG. 12 shows the effect of neuraminidase on S trimer binding to A549 ACE2+ cells, as analyzed by flow cytometry and summarized with a bar graph, as described in Example 3. Graph Abbreviations: Control=cells without staining; anti-ACE2 AB=cells stained with anti-human ACE2 antibody; Neu=neuraminidase from Clostridium perfringens.

FIG. 13 provides bar graphs showing the effect on hACE2 binding, Calsepa binding, ECL binding, and BanLec binding, exhibited by SARS-CoV-2 S trimers and RBD protein, as a result of modulating the prevalence of N-glycans on the S trimers and RBD protein, as described in Example 4. * P≤0.05, ** P≤0.01, ***P≤0.001, ****P≤0.0001. Graph Abbreviation: Vehicle=vehicle buffer only.

FIGS. 14A and 14B show the ACE2 Expression on A549 ACE+ cells (see FIG. 14A) and the effect of PNGase F on S trimer binding to A549 ACE2+ cells (see FIG. 14B), as analyzed by flow cytometry and summarized with a bar graph, as described in Example 4. Graph Abbreviations: Control=cells without staining; anti-ACE2 AB=cells stained with anti-human ACE2 antibody.

FIG. 15 provides bar graphs showing the presence of O-glycans on RBD protein, exhibited by PNA bindings, VVL bindings, SNA bindings, and MAL2 bindings before and after neuraminidase treatment, as described in Example 5. * P≤0.05, ** P≤0.01, ***P≤0.001, ****P≤0.0001. Graph Abbreviations: Vehicle=vehicle buffer only; Neu=neuraminidase from Clostridium perfringens.

FIG. 16 provides bar graphs showing the effect on hACE2 binding and PNA binding, exhibited by SARS-CoV-2 RBD protein, as a result of modulating the prevalence of O-glycans on the RBD protein, as described in Example 5. * P≤0.05, ** P≤0.01, ***P≤0.001, ****P≤0.0001. Graph Abbreviations: Vehicle=vehicle buffer only; Neu=neuraminidase from Clostridium perfringens; Panel=a panel of enzymes used to break down the O-glycans which includes neuraminidase from Clostridium perfringens, α1-2,3,4,6 Fucosidase, β1-4 Galactosidase S, β-N-Acetylglucosaminidase S, and O-Glycosidase.

FIG. 17 shows the effect of D-mannose treatment on sialylation of MCF7 cells, as analyzed by flow cytometry and summarized with bar graphs, as described in Example 6. * P≤0.05, ** P≤0.01, ***P≤0.001, ****P≤0.0001.

FIG. 18 shows the effect of D-mannose treatment on sialylation of PANC1 cells, as analyzed by flow cytometry and summarized with bar graphs, as described in Example 6. * P≤0.05, ** P≤0.01, ***P≤0.001, ****P≤0.0001.

FIG. 19 shows the effect of D-mannose treatment on cell surface sialylation of A549 cells, as analyzed by flow cytometry and summarized with bar graphs, as described in Example 6. * P≤0.05, ** P≤0.01, ***P≤0.001, ****P≤0.0001.

FIG. 20 shows the effect of D-mannose treatment on sialylation of complex N-glycans of MCF7 cells, as profiled by MALDI-TOF, as described in Example 6.

FIG. 21 shows the effect of D-mannose treatment on sialylation of T antigens (O-glycans) of MCF7 cells, as profiled by MALDI-TOF, as described in Example 6.

FIG. 22 shows the effect of D-mannose treatment on sialylation of T antigens (O-glycans) of PANC1 cells, as profiled by MALDI-TOF, as described in Example 6.

FIG. 23 provides a table (Table. 3) summarizing the mass spectrometry intensity of T antigens, sialyl T antigens, and disialyl T antigens of MCF7 cells and PANC1 cells, as described in Example 6.

FIG. 24 shows the effect of D-mannose treatment on N- and O-glycosylation of LN18 cells, as analyzed by flow cytometry and summarized with bar graphs, as described in Example 7. * P≤0.05, ** P≤0.01, ***P≤0.001, ****P≤0.0001.

FIG. 25 shows the effect of D-mannose treatment on N- and O-glycosylation of MDA-MB-231 cells, as analyzed by flow cytometry and summarized with bar graphs, as described in Example 7. * P≤0.05, ** P≤0.01, ***P≤0.001, ****P≤0.0001.

FIG. 26 shows the effect of 1,4-di-O-acetyl-D-mannopyranose treatment on O-glycosylation of A375 cells, as analyzed by flow cytometry and summarized with bar graphs, as described in Example 7. * P≤0.05, ** P≤0.01, ***P≤0.001, ****P≤0.0001. Graph Abbreviations: D1,4=1,4-di-O-acetyl-D-mannopyranose.

FIG. 27 shows the effect of 1,2,3,4-tetra-O-acetyl-D-mannopyranose treatment on N- and O-glycosylation of MCF7 cells, as analyzed by flow cytometry and summarized with bar graphs, as described in Example 7. * P≤0.05, ** P≤0.01, ***P≤0.001, ****P≤0.0001. Graph Abbreviations: D1,2,3,4=1,2,3,4-tetra-O-acetyl-D-mannopyranose.

FIG. 28 is a plot showing the cytotoxicity effect of 1,4-di-O-acetyl-D-mannopyranose treatment on A375 cells, as analyzed by a cell viability assay, as described in Example 7.

FIG. 29 is a plot showing the cytotoxicity effect of 1,2,3,4-tetra-O-acetyl-D-mannopyranose treatment on MCF7 cells, as analyzed by a cell viability assay, as described in Example 7.

FIG. 30 provides bar graphs showing bindings of ECL, PNA, MALL MAL2, SNA, RSL and hACE2, exhibited by SARS-CoV-2 S1 protein expressed in the HEK293F cells treated with or without D-mannose, as described in Example 8. * P≤0.05, ** P≤0.01, ***P≤0.001, ****P≤0.0001. Graph Abbreviations: Vehicle=vehicle buffer only; S1(−DM)=SARS-CoV-2 S1 protein expressed in the HEK293F cells without D-mannose treatment; S1(+DM)=SARS-CoV-2 S1 protein expressed in the HEK293F cells with D-mannose treatment.

### DETAILED DESCRIPTION OF THE INVENTION

The present invention provides methods for ameliorating and/or preventing coronavirus-related infections (e.g., diseases or disorders), such as a SARS-CoV-2 infection or more specifically COVID-19, in a subject.

### Definitions

As used herein, the terms “coronavirus infection” and “coronavirus-related infection” can be used interchangeably and refer to a disease or disorder resulting from invasion and multiplication of a coronavirus in the body of a subject.

As used herein, the term “coronavirus” refers to any member of the Coronavirinae subfamily which belongs to the family Coronaviridae and the order Nidovirales. The coronavirus can exist as any one of the genera of alphacoronaviruses, betacoronaviruses, gammacoronaviruses, and deltacoronaviruses. In some embodiments, the coronavirus is SARS-CoV-2 (severe acute respiratory syndrome coronavirus 2), SARS-CoV (severe acute respiratory syndrome coronavirus), or MERS-CoV (Middle East respiratory syndrome (MERS) coronavirus).

As used herein, the term “host cell” refers to the cells that constitute the human body that will be or have been infected by coronavirus.

As used herein, the term “viral progeny” refers to the viruses generated from the parental viruses within a host cell.

As used herein, the term “subject” refers to a mammal (e.g., human, rat, mouse, cat, dog, cow, pig, sheep, horse, goat, rabbit), preferably a human, in need of prevention and/or treatment of a coronavirus infection. In some embodiments, the subject may be a non-human animal in a veterinary context.

As used herein, the term “therapeutically effective amount” refers to the amount of the sugar or the derivative thereof (e.g., D-mannose) necessary to alleviate or prevent one or more signs and/or symptoms of the treated subject, whether by inducing regression or elimination of such signs and/or symptoms or by inhibiting the progression of such signs and/or symptoms. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); Goodman & Gilman's The Pharmacological Basis of Therapeutics, 11^{th} Edition (McGraw-Hill, 2006); and Remington: The Science and Practice of Pharmacy, 22^{nd} Edition, (Pharmaceutical Press, London, 2012)).

As used herein, the term “treatment” refers to the administration of the sugar or the derivative thereof (e.g., D-mannose) to a subject having one or more signs and/or symptoms of a coronavirus infection.

As used herein, the term “preventing” refers to the prophylactic administration of the sugar or the derivative thereof (e.g., D-mannose) to a subject who is at risk of a coronavirus infection to inhibit the manifestation of a disease or infection in the body of the subject.

As used herein, the term “ameliorating” refers to the administration of the sugar or the derivative thereof (e.g., D-mannose) to a subject having one or more signs and/or symptoms of a coronavirus infection to induce regression or elimination of one or more signs and/or symptoms or to inhibit the progression of one or more signs and/or symptoms.

As used herein, the term “host metabolism” refers to a set of chemical interactions between genes and their products (e.g., proteins) within host cells, resulting in the formation or change of molecules for cellular processes (e.g., alcohol metabolism, carbohydrate metabolism, sugar metabolism, cell cycle metabolism, mitosis metabolism, lipid and fatty acid metabolism, nucleotide and nucleoside metabolism, peptide hormone metabolism, protein and amino acid metabolism, steroid metabolism, vitamin and coenzyme metabolism, and iron, nitric oxide, nitrogen, reversible hydration of carbon dioxide, selenium, sulfur, benzo(a)pyrene, and porphyrin metabolism).

As used herein, the “sign” or “symptom” of a coronavirus infection refer to the survival or proliferation of virus in the body of the subject determined and quantified by the molecular assays for detection of viral nucleic acids (e.g., reverse transcription-polymerase chain reaction) and/or serological and immunological assays (e.g., enzyme-linked immunosorbent assay). The “sign” or “symptoms” of a coronavirus infection also refers to a sign or symptom which is secondary to the viral infection in the body of a subject. For example, the sign or symptom can include fever, feverish/chills, cough, sore throat, runny or stuffy nose, sneezing, muscle or body aches, joint and/or bone pain, headaches, fatigue (tiredness), vomiting, diarrhea, respiratory tract infection, chest discomfort, shortness of breath, bronchitis, pneumonia, cognitive impairment, or a combination thereof.

As used herein, the term “prodrug” denotes a compound, which when administered to a subject, e.g., a human, is converted into the sugar or the derivative thereof (e.g., D-mannose).

As used herein, the term “metabolite” denotes a compound, which is formed from the host cell metabolism of the sugar or the derivative thereof in a subject, e.g., a human.

As used herein, the terms “pharmaceutical formulation” and “pharmaceutical composition” can be used interchangeably and each refer to a mixture comprising the sugar or a derivative thereof (e.g., D-mannose) and one or more pharmaceutically acceptable excipients or pharmaceutically acceptable carriers. Non-limiting examples of pharmaceutically acceptable excipients or pharmaceutically acceptable carriers include water, diluents, salts, buffers, pH adjusters, stabilizers, solubilizers, solvents, and preservatives.

### Methods of Treatment

In some embodiments, the invention provides a method of ameliorating and/or preventing a coronavirus infection in a subject, the method comprising administering to the subject a therapeutically effective amount of a sugar or a derivative thereof selected from D-mannose, fructose, neuraminic acid, mannosamine, glucosamine, galactosamine, a metabolite thereof, a prodrug thereof, or a combination thereof.

The method comprises ameliorating and/or preventing a coronavirus infection in a subject. The coronavirus infection can be any disease or disorder resulting from invasion and multiplication of a coronavirus in the body of a subject. The coronavirus can exist as any one of the genera of alphacoronaviruses, betacoronaviruses, gammacoronaviruses, and deltacoronaviruses. For example, the coronavirus infection can be a severe acute respiratory syndrome coronavirus 2 (SARS-CoV-2) related infection, a severe acute respiratory syndrome coronavirus (SARS-CoV) related infection, or a Middle East respiratory syndrome (MERS) related infection. In certain embodiments, the coronavirus infection is a severe acute respiratory syndrome coronavirus 2 (SARS-CoV-2) related infection, and more particularly, COVID-19.

The sugar or a derivative thereof can be any suitable compound selected from D-mannose, fructose, neuraminic acid, mannosamine, glucosamine, galactosamine, a metabolite thereof, a prodrug thereof, or a combination thereof. In other words, the sugar or a derivative thereof can comprise D-mannose, fructose, neuraminic acid, mannosamine, glucosamine, galactosamine, or a portion thereof. For example, the sugar or a derivative thereof can be D-mannose, mannose-6-phosphate (Man-6-P), fructose-6-phosphate (Fruc-6-P), uridine 5′-diphospho-N-acetylglucosamine (UDP-GlcNAc), mannose-1-phosphate (Man-1-P), guanosine diphosphate mannose (GDP-Man), N-acetylglucosamine (GlcNAc), N-acetylgalactosamine (GalNAc), uridine 5′-diphospho-N-acetylgalactosamine (UDP-GalNAc), N-acetyl-D-mannosamine (ManNAc), N-acetyl-D-mannosamine-6-phosphate (ManNAc-6-P), N-acetylneuraminic-acid-9-phosphate (Neu5Ac-9-P), N-acetylneuraminic acid (Neu5Ac), cytidine-5′-monophospho-N-acetylneuraminic acid (CMP-Neu5Ac), acylated D-mannose, a metabolite thereof, a prodrug thereof, or a combination thereof.

In some embodiments, the sugar or a derivative thereof is D-mannose, mannose-6-phosphate (Man-6-P), fructose-6-phosphate (Fruc-6-P), uridine 5′-diphospho-N-acetylglucosamine (UDP-GlcNAc), mannose-1-phosphate (Man-1-P), guanosine diphosphate mannose (GDP-Man), N-acetylglucosamine (GlcNAc), N-acetylgalactosamine (GalNAc), uridine 5′-diphospho-N-acetylgalactosamine (UDP-GalNAc), N-acetyl-D-mannosamine (ManNAc), N-acetyl-D-mannosamine-6-phosphate (ManNAc-6-P), N-acetylneuraminic-acid-9-phosphate (Neu5Ac-9-P), N-acetylneuraminic acid (Neu5Ac), cytidine-5′-monophospho-N-acetylneuraminic acid (CMP-Neu5Ac), acylated D-mannose, or a combination thereof.

In certain embodiments, the sugar or a derivative thereof is D-mannose, a metabolite thereof, a prodrug thereof, or a combination thereof. For example, the sugar or a derivative thereof can be an acylated D-mannose of the formula:

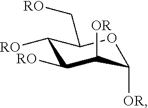

wherein each R independently is hydrogen or

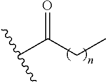

and n is an integer from 0 to 17 (e.g., 1, 2, 3, 4, 5, 6, 7, 8, 9, 10, 11, 12, 13, 14, 15, 16, or 17), and at least one R is

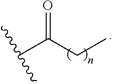

In preferred embodiments, the sugar or a derivative thereof is D-mannose. In some embodiments, the acylated D-mannose comprises 1-O-acetyl-D-mannopyranose, 2-O-acetyl-D-mannopyranose, 3-O-acetyl-D-mannopyranose, 4-O-acetyl-D-mannopyranose, 6-O-acetyl-D-mannopyranose, 1,2-di-O-acetyl-D-mannopyranose, 1,3-di-O-acetyl-D-mannopyranose, 1,4-di-O-acetyl-D-mannopyranose, 1,6-di-O-acetyl-D-mannopyranose, 2,3-di-O-acetyl-D-mannopyranose, 2,4-di-O-acetyl-D-mannopyranose, 2,6-di-O-acetyl-D-mannopyranose, 3,4-di-O-acetyl-D-mannopyranose, 3,6-di-O-acetyl-D-mannopyranose, 4,6-di-O-acetyl-D-mannopyranose, 1,2,3-tri-O-acetyl-D-mannopyranose, 1,2,4-tri-O-acetyl-D-mannopyranose, 1,2,6-tri-O-acetyl-D-mannopyranose, 1,3,4-tri-O-acetyl-D-mannopyranose, 1,3,6-tri-O-acetyl-D-mannopyranose, 1,4,6-tri-O-acetyl-D-mannopyranose, 2,3,4-tri-O-acetyl-D-mannopyranose, 2,3,6-tri-O-acetyl-D-mannopyranose, 2,4,6-tri-O-acetyl-D-mannopyranose, 3,4,6-tri-O-acetyl-D-mannopyranose, 1,2,3,4-tetra-O-acetyl-D-mannopyranose, 1,2,3,6-tetra-O-acetyl-D-mannopyranose, 1,2,4,6-tetra-O-acetyl-D-mannopyranose, 1,3,4,6-tetra-O-acetyl-D-mannopyranose, 2,3,4,6-tetra-O-acetyl-D-mannopyranose, 1,2,3,4,6-penta-O-acetyl-D-mannopyranose, or a combination thereof.

In other embodiments, the sugar or a derivative thereof is mannose-6-phosphate (Man-6-P), fructose-6-phosphate (Fruc-6-P), uridine 5′-diphospho-N-acetylglucosamine (UDP-GlcNAc), mannose-1-phosphate (Man-1-P), guanosine diphosphate mannose (GDP-Man), N-acetylglucosamine (GlcNAc), N-acetylgalactosamine (GalNAc), uridine 5′-diphospho-N-acetylgalactosamine (UDP-GalNAc), N-acetyl-D-mannosamine (ManNAc), N-acetyl-D-mannosamine-6-phosphate (ManNAc-6-P), N-acetylneuraminic-acid-9-phosphate (Neu5Ac-9-P), N-acetylneuraminic acid (Neu5Ac), cytidine-5′-monophospho-N-acetylneuraminic acid (CMP-Neu5Ac), or a combination thereof.

The present invention further provides a method of ameliorating and/or preventing a coronavirus infection (e.g., a SARS-COV-2 infection) in a subject, the method comprising administering to the subject a therapeutically effective amount of D-mannose, a metabolite thereof, a prodrug thereof, or a combination thereof.

Mannose is a sugar monomer of the aldohexose family. There are two mirror isomers of mannose, D- and L-mannose; however, L-mannose does not naturally occur and is not normally used in biological systems (Sharma et al., Biochem. Biophys. Res. Commun. 453, 220 (2014)). In contrast, D-mannose is widely present in the biological systems. D-mannose exists is a C-2 epimer of glucose and exists as α- or β-anomer of the pyranose. The α-anomer is sweet (sucrose-like) and the β-anomer is bitter (quinine-like) (Stewart et al., Nature, 234, 220 (1971); Steinhardt et al., Science, 135, 367-368 (1962)). Mannose can be found in microbes, plants and animals, with free mannose existing in many fruits. Mannose can exist in homo- or hetero-polymers such as mannans and galactomannans. However, many of these polysaccharides only provide a limited amount of bio-available D-mannose for mammalian glycan synthesis as they are not degraded in gastrointestinal track (Sharma et al., Biochem. Biophys. Res. Commun., 453, 220 (2014)). In the human body, D-mannose can be produced from glucose or converted back into glucose.

D-mannose enters into cells through facilitated diffusion by hexose transport facilitators—SLC2A group (GLUT). These transport facilitators are primarily present on the plasma membrane. After entering into the cells, D-mannose is phosphorylated by hexokinase (HK) to produce mannose-6-phosphate (Man-6-P). Man-6-P is either catabolized by phosphomannose isomerase (WI) or directed into N-glycosylation through phosphomannomutase (PMM2). A lower ratio of MPI to PMM2 leads to glycosylation pathway, while a higher ratio leads to catabolism (Sharma et al., J. Biol. Chem., 286(12): 10193-10200 (2011)). PMM2 converts Man-6-P into mannose-1-phosphate (Man-1-P), which is incorporated into a few intermediates critical for N-glycosylation, O-glycosylation, C-mannosylation, and glycosylphosphatidylinositol (GPI) anchor synthesis. These intermediates include GDP-mannose (GDP-Man), GDP-fucose, and dolichol phosphate mannose (Dol-P-Man) (Sharma et al., Biochem. Biophys. Res. Commun. 453, 220 (2014)). There is considerable crosstalk between mannose and other sugars (e.g., glucose) in human metabolism. It has been reported that Man-6-P, one of the major products from mannose metabolism, can inhibit three critical enzymes involving glucose metabolism: hexokinases (HK), phosphoglucose isomerase (PGI) and glucose-6-phosphate dehydrogenase (DeRossi et al., J. Biol. Chem., 281, 5916-5927 (2006)). After D-mannose is phosphorylated into Man-6-P by HK. Man-6-P can also be isomerized into fructose-6-phosphate by phosphomannose isomerase (PMI), therefore entering into glycolysis. Both PMI and PGI can also generate Man-6-P from glucose-6-phosphate (Gonzalez et al., Nature, 563, 719-723 (2018)). Therefore, D-mannose is bioavailable and actively participates in the metabolism regulations.

Without wishing to be bound by any particular theory, it is believed that administering the therapeutically effective amount of the sugar or a derivative thereof (e.g., D-mannose) prevents entry of the virus particle into the host cell and inhibits virus multiplication upon infection by upregulating sialylation of a glycan, downregulating N-glycosylation, and/or downregulating O-glycosylation of a virus particle of the coronavirus. It is believed that the sugar or a derivative thereof (e.g., D-mannose) alters host cell glycosylation pathways to upregulate sialylation, downregulate N-glycosylation, and/or downregulate O-glycosylation, thereby generating more sialylated glycans, less N-glycans and/or less O-glycans on the virus particle of the coronavirus upon entering into host cells being treated with the sugar or a derivative thereof (e.g., D-mannose). Accordingly, the viral progeny are less infectious as more sialic acids are capped at the terminus of glycoconjugates on or within the virus particle (e.g., glycoconjugates that impact the viral life cycle), and less N-glycans and/or O-glycans are present on or within the virus particle (e.g., glycoconjugates that impact the viral life cycle).

Thus, in some embodiments, the invention provides a method of ameliorating and/or preventing a coronavirus infection in a subject, the method comprising upregulating sialylation of a glycan of a virus particle of the coronavirus. The method (e.g., administering the therapeutically effective amount of the sugar or a derivative thereof) can upregulate sialylation of any suitable glycan of the virus particle of the coronavirus. For example, the method may comprise upregulating sialylation of a glycan of a spike protein, a membrane glycoprotein, an envelope protein, a nucleocapsid protein, a phospholipid, or a combination thereof of the virus particle of the coronavirus. In certain embodiments, the method comprises upregulating sialylation of a glycan of a spike protein of the virus particle of the coronavirus.

The glycan can be any suitable glycan of the virus particle of the coronavirus. For example the glycan can be an N-linked glycan, an O-linked glycan, a glycosphingolipid glycan, or a combination thereof. Common N-linked glycans, O-linked glycans, and glycosphingolipid glycans will be readily apparent to a person of ordinary skill in the art. In some embodiments, the glycan is an N-linked glycan selected from high-mannose N-glycans, complex N-glycans, hybrid N-glycans, or a combination thereof. In other embodiments, the glycan is an O-linked glycan selected from O-GalNAc O-glycans, O-GlcNAc O-glycans, O-Mannose O-glycans, O-Galactose O-glycans, O-Fucose O-glycans, O-Glucose O-glycans, or a combination thereof. In certain embodiments, the glycan is a glycosphingolipid glycan selected from cerebrosides, gangliosides, globosides, and a combination thereof.

As used herein, sialylation refers to the addition or reduction (i.e., abundance) of one or more sialic acid residues, which are covalently bound to a glycan of the virus particle of the coronavirus. The one or more sialic acid residues can be bound to the glycan of the virus particle of the coronavirus by any suitable means. For example, the one or more sialic acids can be bound to the glycan of the virus particle of the coronavirus as an alpha (2,3) linkage and/or an alpha (2,6) linkage to one or more sugar residues (e.g., glucose, fucose, galactose, mannose, etc.) of the glycan. Alternatively, or additionally, the one or more sialic acids can be bound to the glycan of the virus particle as an alpha (2,8) linkage and/or an alpha (2,9) linkage to one or more sialic acids of the glycan. In other words, sialylation can refer to the addition or reduction (i.e., abundance) of one sialic acid (monosialylation) or the addition or reduction (i.e., abundance) of more than one sialic acid (polysialylation).

In some embodiments, the method comprises upregulating sialylation of a glycan of a virus particle of the coronavirus. As used herein, upregulating sialylation refers to an increase in the abundance of sialic acids covalently bound to the glycan of the virus particle of the coronavirus relative to the level of sialylation intrinsically present in the virus particle of the coronavirus (e.g., SARS-CoV-2). For example, upregulating sialylation can refer to an increase in sialic acids covalently bound to a glycan of a viral progeny of the coronavirus relative to the level of sialic acids covalently bound to a glycan of a virus particle of the parental coronavirus in the subject prior to being treated with the sugar or the derivative thereof. Similarly, upregulating sialylation can refer to an increase in sialic acids covalently bound to a glycan of viral progeny of the coronavirus relative to the level of sialic acids covalently bound to a glycan of virus particles of the parental coronavirus in the subject prior to being treated with the sugar or the derivative thereof. In other words, upregulating sialylation can refer to an increase in sialic acids covalently bound to the glycan of the virus particle of the coronavirus relative to the level of sialylation present in a virus particle of the coronavirus in the subject prior to being treated with the sugar or the derivative thereof.

The level of sialylation can be upregulated by any suitable amount. For example, the sialylation of the virus particle of the coronavirus can be upregulated by at least 4% (e.g., at least 8%, at least 12%, at least 16%, or at least 20%) relative to a virus particle of the coronavirus in the subject prior to being treated with the sugar or the derivative thereof. In some embodiments, the sialylation of the virus particle of the coronavirus can be upregulated by as much as 50% (e.g., as much as 40%, as much as 30%, or as much as 20%) relative to a virus particle of the coronavirus in the subject prior to being treated with the sugar or the derivative thereof. Thus, the sialylation of the virus particle of the coronavirus can be increased from 4% to 50%, for example, from 4% to 40%, from 4% to 30%, from 4% to 20%, from 8% to 50%, from 4% to 40%, from 4% to 30%, from 4% to 20%, from 12% to 50%, from 12% to 40%, from 12% to 30%, from 12% to 20%, from 16% to 50%, from 16% to 40%, from 16% to 30%, from 16% to 20%, from 20% to 50%, from 20% to 40%, or from 20% to 30%.

In some embodiments, the sialylation of the spike (S) protein of the virus particle of the coronavirus is upregulated by at least 4% (e.g., at least 8%, at least 12%, at least 16%, or at least 20%) relative to a virus particle of the coronavirus in the subject prior to being treated. In some embodiments, the sialylation of the spike (S) protein of the virus particle of the coronavirus is upregulated by as much as 50% (e.g., as much as 40%, as much as 30%, or as much as 20%) relative to a virus particle of the coronavirus in the subject prior to being treated. Thus, the sialylation of the spike (S) protein of the virus particle of the coronavirus can be increased from 4% to 50%, for example, from 4% to 40%, from 4% to 30%, from 4% to 20%, from 8% to 50%, from 4% to 40%, from 4% to 30%, from 4% to 20%, from 12% to 50%, from 12% to 40%, from 12% to 30%, from 12% to 20%, from 16% to 50%, from 16% to 40%, from 16% to 30%, from 16% to 20%, from 20% to 50%, from 20% to 40%, or from 20% to 30%.

In some embodiments, the sialylation of an envelope (E) protein of the virus particle of the coronavirus is upregulated by at least 4% (e.g., at least 8%, at least 12%, at least 16%, or at least 20%) relative to a virus particle of the coronavirus in the subject prior to being treated. In some embodiments, the sialylation of an envelope (E) protein of the virus particle of the coronavirus is upregulated by as much as 50% (e.g., as much as 40%, as much as 30%, or as much as 20%) relative to a virus particle of the coronavirus in the subject prior to being treated. Thus, the sialylation of an envelope (E) protein of the virus particle of the coronavirus can be increased from 4% to 50%, for example, from 4% to 40%, from 4% to 30%, from 4% to 20%, from 8% to 50%, from 4% to 40%, from 4% to 30%, from 4% to 20%, from 12% to 50%, from 12% to 40%, from 12% to 30%, from 12% to 20%, from 16% to 50%, from 16% to 40%, from 16% to 30%, from 16% to 20%, from 20% to 50%, from 20% to 40%, or from 20% to 30%.

In some embodiments, the sialylation of a membrane (M) protein of the virus particle of the coronavirus is upregulated by at least 4% (e.g., at least 8%, at least 12%, at least 16%, or at least 20%) relative to a virus particle of the coronavirus in the subject prior to being treated. In some embodiments, the sialylation of a membrane (M) protein of the virus particle of the coronavirus is upregulated by as much as 50% (e.g., as much as 40%, as much as 30%, or as much as 20%) relative to a virus particle of the coronavirus in the subject prior to being treated. Thus, the sialylation of a membrane (M) protein of the virus particle of the coronavirus can be increased from 4% to 50%, for example, from 4% to 40%, from 4% to 30%, from 4% to 20%, from 8% to 50%, from 4% to 40%, from 4% to 30%, from 4% to 20%, from 12% to 50%, from 12% to 40%, from 12% to 30%, from 12% to 20%, from 16% to 50%, from 16% to 40%, from 16% to 30%, from 16% to 20%, from 20% to 50%, from 20% to 40%, or from 20% to 30%.

In some embodiments, the sialylation of a nucleocapsid (N) protein of the virus particle of the coronavirus is upregulated by at least 4% (e.g., at least 8%, at least 12%, at least 16%, or at least 20%) relative to a virus particle of the coronavirus in the subject prior to being treated. In some embodiments, the sialylation of a nucleocapsid (N) protein of the virus particle of the coronavirus is upregulated by as much as 50% (e.g., as much as 40%, as much as 30%, or as much as 20%) relative to a virus particle of the coronavirus in the subject prior to being treated. Thus, the sialylation of a nucleocapsid (N) protein of the virus particle of the coronavirus can be increased from 4% to 50%, for example, from 4% to 40%, from 4% to 30%, from 4% to 20%, from 8% to 50%, from 4% to 40%, from 4% to 30%, from 4% to 20%, from 12% to 50%, from 12% to 40%, from 12% to 30%, from 12% to 20%, from 16% to 50%, from 16% to 40%, from 16% to 30%, from 16% to 20%, from 20% to 50%, from 20% to 40%, or from 20% to 30%.

The degree of sialylation can be determined by any suitable method, many of which will be readily apparent to a person of ordinary skill in the art. For example, the degree of sialylation can be determined using mass spectrometry (MS) analysis and/or lectin-array analysis. The sample for analysis can be the spike (S) protein, envelope (E) protein, membrane (M) protein, and/or nucleocapsid (N) protein of the coronavirus (e.g., SARS-CoV-2) prepared in a specific expression system (e.g., cultured cells treated with the sugar or the derivative thereof). Alternatively, or additionally, the sample for analysis can be (i) virions obtained from a cell culture of a biological sample, (ii) cell extract obtained from the virus (e.g., infected cells), (iii) tissue extract obtained from the virus (e.g., infected tissue), or (iv) a biological sample (e.g., nasopharyngeal swabs, oropharyngeal swabs, sputum, saliva, blood, pleural effusion, urine, feces, and/or anal swabs) from a subject. Using MS analysis, the sample can be enzymatically or chemically digested to release the glycans or glycopeptides, and the resulting glycans and/or glycopeptides can be subjected to MS, liquid chromatography/MS (LC/MS), or LC/MS-MS analysis. The degree of sialylation can be quantified by normalization of the LC or MS peaks to a standard glycan (e.g., a glycan of a virus particle of the coronavirus in the subject prior to being treated). Using lectin-array analysis, the sample can be fluorescently labeled or reacted with an antibody (e.g., anti-SARS-CoV-2 spike antibody) for detection. The sample can be processed with an antibody (e.g., anti-SARS-CoV-2 spike antibody) with a carrier (e.g., a bead or a surface) to enrich protein/s, viral particles, or other analytes for analysis. The degree of sialylation can be reflected by the binding signals of sialic acid-binding lectins (e.g., SNA, MAL-II) and/or the asialogalactose-binding lectins (e.g., ECL, PNA).

Alternatively, or additionally, in some embodiments, the invention provides a method of ameliorating and/or preventing a coronavirus infection in a subject, the method comprising downregulating the degree of N-glycosylation of a virus particle of the coronavirus. The method (e.g., administering the therapeutically effective amount of the sugar or a derivative thereof) can downregulate the degree (i.e., the prevalence) of any suitable N-glycan of the virus particle of the coronavirus. For example, the method may comprise downregulating N-glycosylation of a spike protein, a membrane glycoprotein, an envelope protein, a nucleocapsid protein, or a combination thereof of the virus particle of the coronavirus. In certain embodiments, the method comprises downregulating N-glycosylation of a spike protein of the virus particle of the coronavirus.

The N-glycan can be any suitable N-linked glycan of the virus particle of the coronavirus. For example the N-glycan can be a high-mannose N-glycan, a complex N-glycan, a hybrid N-glycan, or a combination thereof. Common N-linked glycans will be readily apparent to a person of ordinary skill in the art. In some embodiments, the N-glycan is a N-linked glycan selected from high-mannose N-glycans, complex N-glycans, hybrid N-glycans, and a combination thereof.

As used herein, N-glycosylation refers to the addition or reduction (i.e., abundance) of oligosaccharides with a core sequence, Manα1-3(Manα1-6)Manβ1-4GlcNAcβ1-4GlcNAcβ1-Asn-X-Ser/Thr, which are covalently attached to a nitrogen atom (the amide nitrogen) of an asparagine (Asn) residue of a protein of the virus particle of the coronavirus by an N-glycosidic bond. In some embodiments, the one or more N-glycans is attached to a protein of the virus particle of the coronavirus. For example, the one or more N-glycans can be attached to a spike protein, a membrane glycoprotein, an envelope protein, a nucleocapsid protein, or a combination thereof.

In some embodiments, the method comprises downregulating N-glycosylation of a virus particle of the coronavirus. As used herein, downregulating N-glycosylation refers to a decrease in the abundance of one or more N-glycans covalently attached to an asparagine (Asn) residue of a protein of a viral progeny of coronavirus relative to the level of the same N-glycan(s) intrinsically present on a protein of a parental virus of the coronavirus (e.g., SARS-CoV-2). For example, downregulating N-glycosylation can refer to a decrease in N-glycosylation present on or within a viral progeny of the coronavirus relative to the level of the N-glycosylation present on or within a virus particle of the parental coronavirus in the subject prior to being treated with the sugar or the derivative thereof. Similarly, downregulating N-glycosylation can refer to a decrease in N-glycosylation present on or within viral progeny of the coronavirus relative to the level of the N-glycosylation present on or within virus particles of the parental coronavirus in the subject prior to being treated with the sugar or the derivative thereof. In other words, downregulating N-glycosylation can refer to a decrease in one or more N-glycan(s) covalently attached to a protein of a viral progeny of the coronavirus relative to the level of the same N-glycan(s) present on a protein of a parental virus particle of the coronavirus in the subject prior to being treated with the sugar or the derivative thereof. Similarly, downregulating N-glycosylation can refer to a decrease in one or more N-glycan(s) covalently attached to a protein of viral progeny of the coronavirus relative to the level of the same N-glycan(s) present on a protein of parental virus particles of the coronavirus in the subject prior to being treated with the sugar or the derivative thereof.

The level of N-glycosylation can be downregulated by any suitable amount. For example, the N-glycosylation of the virus particle of the coronavirus can be downregulated by at least 4% (e.g., at least 8%, at least 12%, at least 16%, or at least 20%) relative to a virus particle of the coronavirus in the subject prior to being treated with the sugar or the derivative thereof. In some embodiments, the N-glycosylation of the virus particle of the coronavirus can be downregulated by as much as 100% (e.g., as much as 80%, as much as 60%, as much as 40%, or as much as 20%) relative to a virus particle of the coronavirus in the subject prior to being treated with the sugar or the derivative thereof. Thus, the N-glycosylation of the virus particle of the coronavirus can be decreased from 4% to 100%, for example, from 4% to 80%, from 4% to 60%, from 4% to 40%, from 4% to 20%, from 8% to 100%, from 8% to 80%, from 8% to 60%, from 8% to 40%, from 8% to 20%, from 12% to 100%, from 12% to 80%, from 12% to 60%, from 12% to 40%, from 12% to 20%, from 16% to 100%, from 16% to 80%, from 16% to 60%, from 16% to 40%, from 16% to 20%, from 20% to 100%, from 20% to 80%, from 20% to 60%, or from 20% to 40%.

In some embodiments, the N-glycosylation of the spike (S) protein of the virus particle of the coronavirus is downregulated by at least 4% (e.g., at least 8%, at least 12%, at least 16%, or at least 20%) relative to a virus particle of the coronavirus in the subject prior to being treated. In some embodiments, the N-glycosylation of the spike (S) protein of the virus particle of the coronavirus is downregulated by as much as 100% (e.g., as much as 80%, as much as 60%, as much as 40%, or as much as 20%) relative to a virus particle of the coronavirus in the subject prior to being treated. Thus, the N-glycosylation of the spike (S) protein of the virus particle of the coronavirus can be decreased from 4% to 100%, for example, from 4% to 80%, from 4% to 60%, from 4% to 40%, from 4% to 20%, from 8% to 100%, from 8% to 80%, from 8% to 60%, from 8% to 40%, from 8% to 20%, from 12% to 100%, from 12% to 80%, from 12% to 60%, from 12% to 40%, from 12% to 20%, from 16% to 100%, from 16% to 80%, from 16% to 60%, from 16% to 40%, from 16% to 20%, from 20% to 100%, from 20% to 80%, from 20% to 60%, or from 20% to 40%.

In some embodiments, the N-glycosylation of an envelope (E) protein of the virus particle of the coronavirus is downregulated by at least 4% (e.g., at least 8%, at least 12%, at least 16%, or at least 20%) relative to a virus particle of the coronavirus in the subject prior to being treated. In some embodiments, the N-glycosylation of an envelope (E) protein of the virus particle of the coronavirus is downregulated by as much as 100% (e.g., as much as 80%, as much as 60%, as much as 40%, or as much as 20%) relative to a virus particle of the coronavirus in the subject prior to being treated. Thus, the N-glycosylation of an envelope (E) protein of the virus particle of the coronavirus can be decreased from 4% to 100%, for example, from 4% to 80%, from 4% to 60%, from 4% to 40%, from 4% to 20%, from 8% to 100%, from 8% to 80%, from 8% to 60%, from 8% to 40%, from 8% to 20%, from 12% to 100%, from 12% to 80%, from 12% to 60%, from 12% to 40%, from 12% to 20%, from 16% to 100%, from 16% to 80%, from 16% to 60%, from 16% to 40%, from 16% to 20%, from 20% to 100%, from 20% to 80%, from 20% to 60%, or from 20% to 40%.

In some embodiments, the N-glycosylation of a membrane (M) protein of the virus particle of the coronavirus is downregulated by at least 4% (e.g., at least 8%, at least 12%, at least 16%, or at least 20%) relative to a virus particle of the coronavirus in the subject prior to being treated. In some embodiments, the N-glycosylation of a membrane (M) protein of the virus particle of the coronavirus is downregulated by as much as 100% (e.g., as much as 80%, as much as 60%, as much as 40%, or as much as 20%) relative to a virus particle of the coronavirus in the subject prior to being treated. Thus, the N-glycosylation of a membrane (M) protein of the virus particle of the coronavirus can be decreased from 4% to 100%, for example, from 4% to 80%, from 4% to 60%, from 4% to 40%, from 4% to 20%, from 8% to 100%, from 8% to 80%, from 8% to 60%, from 8% to 40%, from 8% to 20%, from 12% to 100%, from 12% to 80%, from 12% to 60%, from 12% to 40%, from 12% to 20%, from 16% to 100%, from 16% to 80%, from 16% to 60%, from 16% to 40%, from 16% to 20%, from 20% to 100%, from 20% to 80%, from 20% to 60%, or from 20% to 40%.

In some embodiments, the N-glycosylation of a nucleocapsid (N) protein of the virus particle of the coronavirus is downregulated by at least 4% (e.g., at least 8%, at least 12%, at least 16%, or at least 20%) relative to a virus particle of the coronavirus in the subject prior to being treated. In some embodiments, the N-glycosylation of a nucleocapsid (N) protein of the virus particle of the coronavirus is downregulated by as much as 100% (e.g., as much as 80%, as much as 60%, as much as 40%, or as much as 20%) relative to a virus particle of the coronavirus in the subject prior to being treated. Thus, the N-glycosylation of a nucleocapsid (N) protein of the virus particle of the coronavirus can be decreased from 4% to 100%, for example, from 4% to 80%, from 4% to 60%, from 4% to 40%, from 4% to 20%, from 8% to 100%, from 8% to 80%, from 8% to 60%, from 8% to 40%, from 8% to 20%, from 12% to 100%, from 12% to 80%, from 12% to 60%, from 12% to 40%, from 12% to 20%, from 16% to 100%, from 16% to 80%, from 16% to 60%, from 16% to 40%, from 16% to 20%, from 20% to 100%, from 20% to 80%, from 20% to 60%, or from 20% to 40%.

The degree of N-glycosylation can be determined by any suitable method, many of which will be readily apparent to a person of ordinary skill in the art. For example, the degree of N-glycosylation can be determined using mass spectrometry (MS) analysis and/or lectin-array analysis. The sample for analysis can be the spike (S) protein, envelope (E) protein, membrane (M) protein, and/or nucleocapsid (N) protein of the coronavirus (e.g., SARS-CoV-2) prepared in a specific expression system (e.g., cultured cells treated with the sugar or the derivative thereof). Alternatively, or additionally, the sample for analysis can be (i) virions obtained from a cell culture of a biological sample, (ii) cell extract obtained from the virus (e.g., infected cells), (iii) tissue extract obtained from the virus (e.g., infected tissue), or (iv) a biological sample (e.g., nasopharyngeal swabs, oropharyngeal swabs, sputum, saliva, blood, pleural effusion, urine, feces, and/or anal swabs) from a subject. Using MS analysis, the sample can be enzymatically or chemically digested to release the glycans or glycopeptides, and the resulting glycans and/or glycopeptides can be subjected to MS, liquid chromatography/MS (LC/MS), or LC/MS-MS analysis. The degree of N-glycosylation can be quantified by normalization of the LC or MS peaks to a standard glycan (e.g., a glycan of a virus particle of the coronavirus in the subject prior to being treated). Using lectin-array analysis, the sample can be fluorescently labeled or reacted with an antibody (e.g., anti-SARS-CoV-2 spike antibody) for detection. The sample can be processed with an antibody (e.g., anti-SARS-CoV-2 spike antibody) with a carrier (e.g., a bead or a surface) to enrich protein(s), viral particles, and/or other analytes for analysis. The degree of N-glycosylation can be reflected by the binding signals of N-glycan recognizing lectins (e.g., Calsepa, ECL, BanLec) and/or the sialic acid-binding lectins (e.g., SNA, MAL2) with or without neuraminidase (e.g., neuraminidase from Clostridium perfringens) treatment.

Alternatively, or additionally, in some embodiments, the invention provides a method of ameliorating and/or preventing a coronavirus infection in a subject, the method comprising downregulating the degree of O-glycosylation of a virus particle of the coronavirus. The method (e.g., administering the therapeutically effective amount of the sugar or a derivative thereof) can downregulate the degree (i.e., the prevalence) of any suitable O-glycan of the virus particle of the coronavirus. For example, the method may comprise downregulating O-glycosylation of a spike protein, a membrane glycoprotein, an envelope protein, a nucleocapsid protein, or a combination thereof of the virus particle of the coronavirus. In certain embodiments, the method comprises downregulating O-glycosylation of a spike protein of the virus particle of the coronavirus.

The O-glycan can be any suitable O-linked glycan of the virus particle of the coronavirus. For example the O-glycan can be O-GalNAc O-glycans, O-GlcNAc O-glycans, O-Mannose O-glycans, O-Galactose O-glycans, O-Fucose O-glycans, O-Glucose O-glycans, or a combination thereof. Common O-linked glycans will be readily apparent to a person of ordinary skill in the art. In some embodiments, the O-glycan is an O-linked glycan selected from Tn antigen, STn antigen, core 1 type of O-glycans, core 2 type of O-glycans, core 3 type of O-glycans, core 4 type of O-glycans, core 5 type of O-glycans, core 6 type of O-glycans, core 7 type of O-glycans, and a combination thereof.

As used herein, O-glycosylation refers to the addition or reduction (i.e., abundance) of oligosaccharides with a N-Acetylgalactosamine (GalNAc) residue covalently attached to the oxygen atom of a serine (Ser) or threonine (Thr) residue of a protein of the virus particle of the coronavirus. For example, the one or more O-glycans can be attached to a spike protein, a membrane glycoprotein, an envelope protein, a nucleocapsid protein, or a combination thereof.

In some embodiments, the method comprises downregulating O-glycosylation of a virus particle of the coronavirus. As used herein, downregulating O-glycosylation refers to a decrease in the abundance of one or more O-glycans covalently attached to a serine (Ser) or threonine (Thr) residue of a protein of a viral progeny of coronavirus relative to the level of the same O-glycan(s) intrinsically present on a protein of a parental virus of the coronavirus (e.g., SARS-CoV-2). For example, downregulating O-glycosylation can refer to a decrease in O-glycosylation present on or within a viral progeny of the coronavirus relative to the level of the O-glycosylation present on or within a virus particle of the parental coronavirus in the subject prior to being treated with the sugar or the derivative thereof. Similarly, downregulating O-glycosylation can refer to a decrease in O-glycosylation present on or within viral progeny of the coronavirus relative to the level of the O-glycosylation present on or within virus particles of the parental coronavirus in the subject prior to being treated with the sugar or the derivative thereof. In other words, downregulating O-glycosylation can refer to a decrease in one or more O-glycan(s) covalently attached to a protein of a viral progeny of the coronavirus relative to the level of the same O-glycan(s) present on a protein of a parental virus particle of the coronavirus in the subject prior to being treated with the sugar or the derivative thereof. Similarly, downregulating O-glycosylation can refer to a decrease in one or more O-glycan(s) covalently attached to a protein of viral progeny of the coronavirus relative to the level of the same O-glycan(s) present on a protein of parental virus particles of the coronavirus in the subject prior to being treated with the sugar or the derivative thereof.

The level of O-glycosylation can be downregulated by any suitable amount. For example, the O-glycosylation of the virus particle of the coronavirus can be downregulated by at least 4% (e.g., at least 8%, at least 12%, at least 16%, or at least 20%) relative to a virus particle of the coronavirus in the subject prior to being treated with the sugar or the derivative thereof. In some embodiments, the O-glycosylation of the virus particle of the coronavirus can be downregulated by as much as 100% (e.g., as much as 80%, as much as 60%, as much as 40%, or as much as 20%) relative to a virus particle of the coronavirus in the subject prior to being treated with the sugar or the derivative thereof. Thus, the O-glycosylation of the virus particle of the coronavirus can be decreased from 4% to 100%, for example, from 4% to 80%, from 4% to 60%, from 4% to 40%, from 4% to 20%, from 8% to 100%, from 8% to 80%, from 8% to 60%, from 8% to 40%, from 8% to 20%, from 12% to 100%, from 12% to 80%, from 12% to 60%, from 12% to 40%, from 12% to 20%, from 16% to 100%, from 16% to 80%, from 16% to 60%, from 16% to 40%, from 16% to 20%, from 20% to 100%, from 20% to 80%, from 20% to 60%, or from 20% to 40%.

In some embodiments, the O-glycosylation of the spike (S) protein of the virus particle of the coronavirus is downregulated by at least 4% (e.g., at least 8%, at least 12%, at least 16%, or at least 20%) relative to a virus particle of the coronavirus in the subject prior to being treated. In some embodiments, the O-glycosylation of the spike (S) protein of the virus particle of the coronavirus is downregulated by as much as 100% (e.g., as much as 80%, as much as 60%, as much as 40%, or as much as 20%) relative to a virus particle of the coronavirus in the subject prior to being treated. Thus, the O-glycosylation of the spike (S) protein of the virus particle of the coronavirus can be decreased from 4% to 100%, for example, from 4% to 80%, from 4% to 60%, from 4% to 40%, from 4% to 20%, from 8% to 100%, from 8% to 80%, from 8% to 60%, from 8% to 40%, from 8% to 20%, from 12% to 100%, from 12% to 80%, from 12% to 60%, from 12% to 40%, from 12% to 20%, from 16% to 100%, from 16% to 80%, from 16% to 60%, from 16% to 40%, from 16% to 20%, from 20% to 100%, from 20% to 80%, from 20% to 60%, or from 20% to 40%.

In some embodiments, the O-glycosylation of an envelope (E) protein of the virus particle of the coronavirus is downregulated by at least 4% (e.g., at least 8%, at least 12%, at least 16%, or at least 20%) relative to a virus particle of the coronavirus in the subject prior to being treated. In some embodiments, the O-glycosylation of an envelope (E) protein of the virus particle of the coronavirus is downregulated by as much as 100% (e.g., as much as 80%, as much as 60%, as much as 40%, or as much as 20%) relative to a virus particle of the coronavirus in the subject prior to being treated. Thus, the O-glycosylation of an envelope (E) protein of the virus particle of the coronavirus can be decreased from 4% to 100%, for example, from 4% to 80%, from 4% to 60%, from 4% to 40%, from 4% to 20%, from 8% to 100%, from 8% to 80%, from 8% to 60%, from 8% to 40%, from 8% to 20%, from 12% to 100%, from 12% to 80%, from 12% to 60%, from 12% to 40%, from 12% to 20%, from 16% to 100%, from 16% to 80%, from 16% to 60%, from 16% to 40%, from 16% to 20%, from 20% to 100%, from 20% to 80%, from 20% to 60%, or from 20% to 40%.

In some embodiments, the O-glycosylation of a membrane (M) protein of the virus particle of the coronavirus is downregulated by at least 4% (e.g., at least 8%, at least 12%, at least 16%, or at least 20%) relative to a virus particle of the coronavirus in the subject prior to being treated. In some embodiments, the O-glycosylation of a membrane (M) protein of the virus particle of the coronavirus is downregulated by as much as 100% (e.g., as much as 80%, as much as 60%, as much as 40%, or as much as 20%) relative to a virus particle of the coronavirus in the subject prior to being treated. Thus, the O-glycosylation of a membrane (M) protein of the virus particle of the coronavirus can be decreased from 4% to 100%, for example, from 4% to 80%, from 4% to 60%, from 4% to 40%, from 4% to 20%, from 8% to 100%, from 8% to 80%, from 8% to 60%, from 8% to 40%, from 8% to 20%, from 12% to 100%, from 12% to 80%, from 12% to 60%, from 12% to 40%, from 12% to 20%, from 16% to 100%, from 16% to 80%, from 16% to 60%, from 16% to 40%, from 16% to 20%, from 20% to 100%, from 20% to 80%, from 20% to 60%, or from 20% to 40%.

In some embodiments, the O-glycosylation of a nucleocapsid (N) protein of the virus particle of the coronavirus is downregulated by at least 4% (e.g., at least 8%, at least 12%, at least 16%, or at least 20%) relative to a virus particle of the coronavirus in the subject prior to being treated. In some embodiments, the O-glycosylation of a nucleocapsid (N) protein of the virus particle of the coronavirus is downregulated by as much as 100% (e.g., as much as 80%, as much as 60%, as much as 40%, or as much as 20%) relative to a virus particle of the coronavirus in the subject prior to being treated. Thus, the O-glycosylation of a nucleocapsid (N) protein of the virus particle of the coronavirus can be decreased from 4% to 100%, for example, from 4% to 80%, from 4% to 60%, from 4% to 40%, from 4% to 20%, from 8% to 100%, from 8% to 80%, from 8% to 60%, from 8% to 40%, from 8% to 20%, from 12% to 100%, from 12% to 80%, from 12% to 60%, from 12% to 40%, from 12% to 20%, from 16% to 100%, from 16% to 80%, from 16% to 60%, from 16% to 40%, from 16% to 20%, from 20% to 100%, from 20% to 80%, from 20% to 60%, or from 20% to 40%.

The degree of O-glycosylation can be determined by any suitable method, many of which will be readily apparent to a person of ordinary skill in the art. For example, the degree of O-glycosylation can be determined using mass spectrometry (MS) analysis and/or lectin-array analysis. The sample for analysis can be the spike (S) protein, envelope (E) protein, membrane (M) protein, and/or nucleocapsid (N) protein of the coronavirus (e.g., SARS-CoV-2) prepared in a specific expression system (e.g., cultured cells treated with the sugar or the derivative thereof). Alternatively, or additionally, the sample for analysis can be (i) virions obtained from a cell culture of a biological sample, (ii) cell extract obtained from the virus (e.g., infected cells), (iii) tissue extract obtained from the virus (e.g., infected tissue), or (iv) a biological sample (e.g., nasopharyngeal swabs, oropharyngeal swabs, sputum, saliva, blood, pleural effusion, urine, feces, and/or anal swabs) from a subject. Using MS analysis, the sample can be enzymatically or chemically digested to release the glycans or glycopeptides, and the resulting glycans and/or glycopeptides can be subjected to MS, liquid chromatography/MS (LC/MS), or LC/MS-MS analysis. The degree of O-glycosylation can be quantified by normalization of the LC or MS peaks to a standard glycan (e.g., a glycan of a virus particle of the coronavirus in the subject prior to being treated). Using lectin-array analysis, the sample can be fluorescently labeled or reacted with an antibody (e.g., anti-SARS-CoV-2 spike antibody) for detection. The sample can be processed with an antibody (e.g., anti-SARS-CoV-2 spike antibody) with a carrier (e.g., a bead or a surface) to enrich protein(s), viral particles, or other analytes for analysis. The degree of O-glycosylation can be reflected by the binding signals of O-glycan recognizing lectins (e.g., PNA, VVL) and/or the sialic acid-binding lectins (e.g., SNA, MAL2) with or without neuraminidase (e.g., neuraminidase from Clostridium perfringens) treatment.

### Administration and Dosing

For any of the methods described herein, the sugar or a derivative thereof (e.g., D-mannose) can be administered by any suitable means for any suitable duration of time as a single compound or as a pharmaceutical formulation. For example, the sugar or a derivative thereof can be administered orally, rectally, transmucosally, intestinally, parenterally, intramuscularly, subcutaneously, intradermally, intramedullaryly, intrathecally, intraventricularly, intravenously, intraperitoneally, intranasally, intraocularly, inhalationally, insufflationally, topically, cutaneously, transdermally, intra-arterially, or a combination thereof. In some embodiments, the sugar or a derivative thereof (e.g., D-mannose) is administered orally (e.g., as a single compound or as a pharmaceutical formulation). In other embodiments, the sugar or a derivative thereof (e.g., D-mannose) is administered via injection intramuscularly, subcutaneously, or intravenously (e.g., as a single compound or as a pharmaceutical formulation). For example, the sugar or a derivative thereof (e.g., D-mannose) can be administered intravenously via a peripheral vein (e.g., in the hand or arm); the superior vena cava, the inferior vena cava, or within the right atrium of the heart (e.g., a central IV); or into a subclavian, internal jugular, or a femoral vein, and advanced toward the heart until it reaches the superior vena cava or right atrium (e.g., a central venous line).

The methods can include treating coronavirus in a subject comprising administering from about 0.01 mg/kg to about 20 g/kg of the sugar or a derivative thereof (e.g., D-mannose) to the subject. In this regard, the methods can include administering the sugar or a derivative thereof (e.g., D-mannose) to provide a dose of from about 0.01 mg/kg to about 10 g/kg, about 0.01 mg/kg to about 5 g/kg, 0.01 mg/kg to about 2.5 g/kg, about 0.01 mg/kg to about 1 g/kg, about 0.01 mg/kg to about 500 mg/kg, about 0.01 mg/kg to about 100 mg/kg, about 0.1 mg/kg to about 5 g/kg, 0.1 mg/kg to about 2.5 g/kg, about 0.1 mg/kg to about 1 g/kg, about 0.1 mg/kg to about 500 mg/kg, about 0.1 mg/kg to about 100 mg/kg, about 1 mg/kg to about 5 g/kg, 1 mg/kg to about 2.5 g/kg, about 1 mg/kg to about 1 g/kg, about 1 mg/kg to about 500 mg/kg, about 1 mg/kg to about 100 mg/kg, about 10 mg/kg to about 5 g/kg, 10 mg/kg to about 2.5 g/kg, about 10 mg/kg to about 1 g/kg, about 10 mg/kg to about 500 mg/kg, or about 10 mg/kg to about 100 mg/kg.

In some embodiments, administering the therapeutically effective amount of the sugar or a derivative thereof (e.g., D-mannose) achieves a plasma concentration of at least 2 times greater (e.g., at least 3 times greater, at least 4 times greater, at least 5 times greater, at least 10 times greater, or at least 20 times greater) than a plasma concentration of said sugar or said derivative thereof (e.g., D-mannose) prior to administration. In other words, administering the therapeutically effective amount of the sugar or a derivative thereof (e.g., D-mannose) can achieve a plasma concentration of from 2 to 100 times greater, for example, 2 to 50 times greater, 2 to 25 times greater, 3 to 100 times greater, 3 to 50 times greater, 3 to 25 times greater, 4 to 100 times greater, 4 to 50 times greater, 4 to 25 times greater, 5 to 100 times greater, 5 to 50 times greater, 5 to 25 times greater, 10 to 100 times greater, 10 to 50 times greater, or 10 to 25 times greater than the average physiological plasma concentration of said sugar or said derivative thereof (e.g., D-mannose).

The increased plasma concentration of the sugar or a derivative thereof (e.g., D-mannose) can be maintained for any suitable amount of time. For example, the increased plasma concentration of the sugar or a derivative thereof (e.g., D-mannose) can be maintained for a period of 1 hour to 24 hours (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours) before the next administration.

In some embodiments, the sugar or a derivative thereof (e.g., D-mannose) is administered four or more times daily, three times a day, twice daily, once daily, or every other day. In other embodiments, the sugar or a derivative thereof (e.g., D-mannose) is administered from about every 3 to about every 45 days (e.g., about every 3 days, about every 4 days, about every 5 days, about every 6 days, about every 7 days, about every 8 days, about every 9 days, about every 10 days, about every 11 days, about every 12 days, about every 13 days, about every 14 days, about every 15 days, about every 16 days, about every 17 days, about every 18 days, about every 19 days, about every 20 days, about every 21 days, about every 22 days, about every 23 days, about every 24 days, about every 25 days, about every 26 days, about every 27 days, about every 28 days, about every 29 days, about every 30 days, about every 31 days, about every 32 days, about every 33 days, about every 34 days, about every 35 days, about every 36 days, about every 37 days, about every 38 days, about every 39 days, about every 40 days, about every 41 days, about every 42 days, about every 43 days, about every 44 days, or about every 45 days).

In some embodiments, the sugar or a derivative thereof (e.g., D-mannose) is administered during fasting or dietary restriction in which the plasma concentration of D-glucose is (i) less than 60 mg/dL, 65 mg/dL, 70 mg/dL, 75 mg/dL, 80 mg/dL, 85 mg/dL, 90 mg/dL, 95 mg/dL, or 100 mg/dL for non-diabetic, (ii) less than 105 mg/dL, 110 mg/dL, 115 mg/dL, 120 mg/dL, or 125 mg/dL for prediabetic, and (iii) less than 130 mg/dL, 140 mg/dL, 150 mg/dL, 160 mg/dL, 170 mg/dL, 180 mg/dL, 190 mg/dL, 200 mg/dL, 250 mg/dL, or 300 mg/dL for diabetic.

The sugar or a derivative thereof (e.g., D-mannose) can be administered to the subject as an initial loading dose followed by one or more maintenance doses. For example, sugar or a derivative thereof (e.g., D-mannose) can be administered as a loading dose to the subject at about 5 g/kg, about 2.5 g/kg, about 1 g/kg, about 0.5 g/kg, or about 100 mg/kg. The loading dose may be a higher or lower dose than the one or more maintenance doses.

The loading dose may be administered to the patient using a similar or different suitable means than the one or more maintenance doses.

In some embodiments, the sugar or a derivative thereof (e.g., D-mannose) is administered intravenously (e.g., IV infusion). In some embodiments, sugar or a derivative thereof (e.g., D-mannose) is administered to the subject intravenously over about 1 to about 240 minutes. In this regard, the sugar or a derivative thereof (e.g., D-mannose) can be administered over about 5 to about 55 minutes, over about 10 to about 50 minutes, over about 15 to about 45 minutes, over about 20 to about 40 minutes, over about 25 to about 35 minutes, over about 30 minutes to the subject, over about 30 to about 90 minutes, over about 35 to about 85 minutes, over about 40 to about 80 minutes, over about 45 to about 75 minutes, over about 50 to about 70 minutes, over about 55 to about 65 minutes, over about 60 minutes, over about 90 to about 150 minutes, over about 95 to about 145 minutes, over about 100 to about 140 minutes, over about 105 to about 135 minutes, over about 110 to about 130 minutes, over about 115 to about 125 minutes, over about 120 minutes, over about 150 to about 210 minutes, over about 155 to about 205 minutes, over about 160 to about 200 minutes, over about 165 to about 195 minutes, over about 170 to about 190 minutes, over about 175 to about 185 minutes, over about 180 minutes, over about 210 to about 270 minutes, over about 215 to about 265 minutes, over about 220 to about 260 minutes, over about 225 to about 255 minutes, over about 230 to about 250 minutes, over about 235 to about 245 minutes, or over about 240 minutes.

The sugar or a derivative thereof (e.g., D-mannose) can be administered to the subject for any suitable length of time. For example, the sugar or a derivative thereof (e.g., D-mannose) can be administered to the subject one time or multiple times. If the sugar or a derivative thereof (e.g., D-mannose) is administered multiple times, the sugar or a derivative thereof (e.g., D-mannose) can be administered for a duration of from about 1 day to about month to about 12 months (e.g., from about 1 day to about 6 months, about 1 day to about 3 months, about 1 day to about 1 month, about 1 day to about 4 weeks, about 1 day to about 3 weeks, about 1 day to about 2 weeks, about 1 day to about 1 week, from about 1 week to about 12 months, from about 1 week to about 6 months, about 1 week to about 3 months, about 1 week to about 1 month, about 1 week to about 4 weeks, about 1 week to about 3 weeks, or about 1 week to about 2 weeks).

In an exemplary process for oral administration of the sugar or a derivative thereof, D-mannose is orally administered to a subject (e.g., a human) by a regimen effective to inhibit virus multiplication in host cells and prevent viral S protein and ACE2 receptor interaction upon infection. The exemplary regimen includes administering a therapeutically effective amount of D-mannose to a subject by a single dose or multiple doses, for example, by orally ingesting 0.5 grams D-mannose/kg body weight/day by a single dose or orally ingesting 5 doses per day with a total of 1.0 gram D-mannose/kg body weight. The exemplary regimen also includes a sufficient frequency and a duration of the treatment, for example, ingesting 0.5 grams D-mannose/kg body weight/day by a single does for 12 days or ingesting 0.5 grams D-mannose/kg body weight/day, every other day for 14 days.

In an exemplary process for parenteral administration of the sugar or a derivative thereof, D-mannose is parenterally administered to a subject (e.g., a human) by a regimen effective to inhibit virus multiplication in host cells and prevent viral S protein and ACE2 receptor interaction upon infection. The exemplary regimen includes administering a therapeutically effective amount of D-mannose to a subject by a rapid infusion or a prolonged infusion by an injection device. In a non-limiting aspect, the “rapid infusion” refers to the 0.5 to 10.0 grams of D-mannose/kg body weight from a pharmaceutical composition of the present invention is administered into an antecubital vein of a subject (e.g., a human) through an injection device in 3, 4, 5, 6, 7, 8, 9, or 10 minutes within 1 day. In a non-limiting aspect, the “prolonged infusion” refers to the 0.5 to 10.0 grams of D-mannose/kg body weight from a pharmaceutical composition of the present invention is administered into an antecubital vein of a subject (e.g., a human) through an injection device in 1, 2, 3, 4, 5, 6, 7, 8, 9, 10, 11, 12, 13, 14, 15, 16, 17, 18, 19, 20, 21, 22, 23, or 24 hours. Depending on the severity of the infection, the frequency and duration of the treatment can be adjusted. In another non-limiting aspect, the pharmaceutical composition of the invention can be intravenously administered, either by rapid or prolonged infusion at day 1, followed by day 2, day 3 and/or thereafter in an amount that can be approximately the same, more or less than the total amount infused at day 1, by either rapid or prolonged infusion, wherein the subsequent administration can be separated by at least 1, 2, 3, 4, 5, 6, 7, 8, 9, 10, 11, 12, 13, or 14 days (e.g., administered at day 1, day 3, day 5 and/or thereafter).

### Pharmaceutical Compositions, Combinations, and Kits

In any of the methods described herein, the sugar or a derivative thereof (e.g., D-mannose) can be administered as a single compound or as a pharmaceutical formulation. When the sugar or a derivative thereof (e.g., D-mannose) is administered as a pharmaceutical formulation, the sugar or a derivative thereof (e.g., D-mannose) can be combined with any suitable pharmaceutically acceptable excipient and/or pharmaceutically acceptable carrier.

For example, the sugar or a derivative thereof (e.g., D-mannose) can be combined with a suitable carrier such as maltodextrin, starch, cellulose, food-grade silica, flow agents, and acidulants (e.g., citric acid, malic acid and/or ascorbic acid). The composition can be prepared in a wet (e.g., emulsions and liquid concentrate) or dry (e.g., tablets, capsules, and powders) form suitable for administration (e.g., oral administration). In an exemplary embodiment, the composition can be prepared as a dry formulation for delivering the sugar or a derivative thereof (e.g., D-mannose) at a dosage of about 1.0 to 10.0 grams (e.g., 1.0 to 10.0 grams of D-mannose per tablet, capsule, or powder pack). In another exemplary embodiment, the composition can be prepared as a wet formulation for delivering the sugar or a derivative thereof (e.g., D-mannose), the wet formulation comprising 10 (wt./vol %) to 90 (wt./vol %) of the sugar or a derivative thereof (e.g., D-mannose) (e.g., 10 to 90 grams of D-mannose prepares in 100 mL of water or other buffering and stabilizing fluids). Other components can be added to enhance the palatability of the composition. For example, the sugar or a derivative thereof can be combined with natural and/or artificial flavors, nutritive and/or non-nutritive sweeteners, salts, acids, or other suitable ingredients.

Alternatively, or additionally, the sugar or a derivative thereof (e.g., D-mannose) can be admixed with a pharmaceutically acceptable carrier or excipient (e.g., sterile water) as 10%, 15%, 20%, 25%, 30%, 35%, 40%, 45%, 50%, 55%, 60%, 65%, 70%, 75%, 80%, 85%, 90%, 95% solution (wt./vol) suitable for administration (e.g., intramuscular, subcutaneous or intravenous). See, e.g., Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.

In some embodiments, the pharmaceutical composition comprising the sugar or a derivative thereof (e.g., D-mannose) is sterile. For example, the composition comprising the sugar or a derivative thereof (e.g., D-mannose) can be sterilized and pyrogen-free as a 5 (wt./vol %) to 90% (wt./vol %) solution in sterile water.

The sugar or a derivative thereof (e.g., D-mannose) can be administered in associated with one or more additional therapeutic agents. As used herein, the phrase “in association with” means that the additional therapeutic agent(s) can be formulated along with the sugar or a derivative thereof (e.g., D-mannose) in a single formulation or can be administered at the same time as a combination therapy. In other words, the sugar or a derivative thereof (e.g., D-mannose) and the additional therapeutic agent(s) can be administered simultaneously or sequentially as a single formulation or as multiple separate formulations in accordance with the administration techniques described herein.

A list of additional therapeutic agents suitable for administration with the sugar or a derivative thereof (e.g., D-mannose) includes, but is not limited to: an isolated recombinant antibody or antigen-binding fragment that specifically binds to a coronavirus spike protein (CoV-S), an isolated recombinant antibody or antigen-binding fragment that specifically binds TMPRSS2, an isolated recombinant antibody or antigen-binding fragment that specifically binds a chemical component of coronavirus, which prevents its entry into the host cells, an anti-inflammatory agent (e.g., sarilumab, tocilizumab, or gimsilumab), an antimalarial agent (e.g., chloroquine or hydroxychloroquine), an anti-viral drug (e.g., cationic steroid antimicrobial, leupeptin, aprotinin, ribavirin, remdesivir, lopinavir-ritonavir, umifenovir, favipiravir, oseltamivir, molnupiravir, nirmatrelvir, ritonavir, or interferon-alpha2b), a vaccine (e.g., whole-pathogen vaccines such as inactivated/killed virus vaccines or a live attenuated virus vaccines, subunit vaccines such as conjugate vaccines, toxoid vaccines, recombinant protein vaccines, virus-like particles vaccines, nanoparticles vaccines, or nucleic acid vaccines such as DNA plasmid vaccines, mRNA vaccines, and recombinant vector vaccines), a supporting agent (e.g., Chinese herbal medicine, botanical extracts, azithromycin, NSAID, zinc, vitamin c, corticosteroids, nitric oxide, epoprostenol, sirolimus, anakinra, nitazoxanide, tizoxanide, niclosamide, ivermectin, colchicine, indomethacin, or thiazolidinediones), any other anti-viral agent (i.e., and agent that provides an increase in survival of a virus-infected animal after administration to the virus-infected animal). Additional therapeutic agents suitable for administration with the sugar or a derivative thereof can be found in the Physicians' Desk Reference 2003 (Thomson Healthcare; 57th edition, Nov. 1, 2002).

The invention also provides a kit comprising the sugar or a derivative thereof (e.g., D-mannose) in association with one or more further therapeutic agents for use in the treatment of a coronavirus infection in a subject. The sugar or a derivative thereof (e.g., D-mannose) and one or more further therapeutic agents can be formulated as a single composition or separately in two or more compositions (e.g., made in a pharmaceutical composition or comes with a pharmaceutically acceptable carrier).

The kit can include the sugar or a derivative thereof (e.g., D-mannose) in a pharmaceutical composition, either in wet or dry format in one container (e.g., a dry formulation in the form of a pill, capsule, or powder pack or a wet formulation in a sterile glass or plastic vial), and a further therapeutic agent, either in wet or dry format in another container (e.g., a dry formulation in the form of a pill, capsule, or powder pack or a wet formulation in a sterile glass or plastic vial). Additionally, or alternatively, the kit can include a combination, including the sugar or a derivative thereof (e.g., D-mannose) in a pharmaceutical composition, either in wet or dry format, in combination with one or more further therapeutic agents, formulated together in a pharmaceutical composition in a single container.

In embodiments where the kit includes a pharmaceutical composition for parenteral administration to a subject, the kit can further include an injection device. The injection device is any device that introduces the sugar or a derivative thereof (e.g., D-mannose) or a pharmaceutical composition comprising the sugar or a derivative thereof (e.g., D-mannose) into the body of the subject (e.g., a human). The injection device may be a syringe prefilled with a pharmaceutical composition such as an auto-injector for rapid infusion. For example, the injection device can include a cylinder or barrel for holding fluid to be injected (e.g., the sugar or a derivative thereof (e.g., D-mannose) or a pharmaceutical composition comprising the sugar or a derivative thereof (e.g., D-mannose)), a needle for piecing skin and/or blood vessels for injection of the fluid, and a plunger for pushing the fluid out through the needle bore. Alternatively, the injection device may be an intravenous (IV) injection device for prolonged infusion. Such devices can include a cannula or trocar/needle that is attached to a tube, which is connected to a bag or reservoir for holding fluid (e.g., the sugar or a derivative thereof (e.g., D-mannose) or a pharmaceutical composition comprising the sugar or a derivative thereof (e.g., D-mannose)) to be introduced into the body of a subject (e.g., a human) through the cannula or trocar/needle. The IV device may be inserted into a peripheral vein (e.g., in the hand or arm); the superior vena cava, inferior vena cava, or within the right atrium of the heart (e.g., a central IV); or into a subclavian, internal jugular, or a femoral vein, and advanced toward the heart until it reaches the superior vena cava or right atrium (e.g., a central venous line).

In some embodiments, the kit further includes a package insert with all the information that aids patients and physicians in using the enclosed pharmaceutical compositions effectively and safely. For example, the following information can be included for a combination of the invention: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information, and patent information.

### Examples of Non-Limiting Embodiments of the Disclosure

Embodiments, including aspects, of the present subject matter described herein may be beneficial alone or in combination, with one or more other aspects or embodiments. Without limiting the foregoing description, certain non-limiting embodiments of the disclosure numbered 1-47 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

### EMBODIMENTS

(1) In embodiment (1) is presented a method of ameliorating and/or preventing a coronavirus infection in a subject, the method comprising administering to the subject a therapeutically effective amount of a sugar or a derivative thereof selected from D-mannose, fructose, neuraminic acid, mannosamine, glucosamine, galactosamine, a metabolite thereof, a prodrug thereof, or a combination thereof.

(2) In embodiment (2) is presented the method of embodiment (1), wherein the sugar or a derivative thereof is D-mannose, mannose-6-phosphate (Man-6-P), fructose-6-phosphate (Fruc-6-P), uridine 5′-diphospho-N-acetylglucosamine (UDP-GlcNAc), mannose-1-phosphate (Man-1-P), guanosine diphosphate mannose (GDP-Man), N-acetylglucosamine (GlcNAc), N-acetylgalactosamine (GalNAc), uridine 5′-diphospho-N-acetylgalactosamine (UDP-GalNAc), N-acetyl-D-mannosamine (ManNAc), N-acetyl-D-mannosamine-6-phosphate (ManNAc-6-P), N-acetylneuraminic-acid-9-phosphate (Neu5Ac-9-P), N-acetylneuraminic acid (Neu5Ac), cytidine-5′-monophospho-N-acetylneuraminic acid (CMP-Neu5Ac), acylated D-mannose, a metabolite thereof, a prodrug thereof, or a combination thereof.

(3) In embodiment (3) is presented the method of embodiment (1) or embodiment (2), wherein the coronavirus infection is a severe acute respiratory syndrome coronavirus 2 (SARS-CoV-2) related infection.

(4) In embodiment (4) is presented the method of embodiment (3), wherein the severe acute respiratory syndrome coronavirus 2 (SARS-CoV-2) related infection is COVID-19.

(5) In embodiment (5) is presented the method of embodiment (1) or embodiment (2), wherein the coronavirus infection is a severe acute respiratory syndrome coronavirus (SARS-CoV) related infection.

(6) In embodiment (6) is presented the method of embodiment (1) or embodiment (2), wherein the coronavirus infection is a Middle East respiratory syndrome (MERS) related infection.

(7) In embodiment (7) is presented the method of any one of embodiments (1)-(6), wherein the sugar or a derivative thereof is administered orally, rectally, transmucosally, intestinally, parenterally, intramuscularly, subcutaneously, intradermally, intramedullaryly, intrathecally, intraventricularly, intravenously, intraperitoneally, intranasally, intraocularly, inhalationally, insufflationally, topically, cutaneously, transdermally, intra-arterially, or a combination thereof.

(8) In embodiment (8) is presented the method of any one of embodiments (1)-(7), wherein the sugar or a derivative thereof is administered orally.

(9) In embodiment (9) is presented the method of any one of embodiments (1)-(7), wherein the sugar or a derivative thereof is administered intravenously.

(10) In embodiment (10) is presented the method of any one of embodiments (1)-(9), wherein the sugar or a derivative thereof is D-mannose, mannose-6-phosphate (Man-6-P), fructose-6-phosphate (Fruc-6-P), uridine 5′-diphospho-N-acetylglucosamine (UDP-GlcNAc), mannose-1-phosphate (Man-1-P), guanosine diphosphate mannose (GDP-Man), N-acetylglucosamine (GlcNAc), N-acetylgalactosamine (GalNAc), uridine 5′-diphospho-N-acetylgalactosamine (UDP-GalNAc), N-acetyl-D-mannosamine (ManNAc), N-acetyl-D-mannosamine-6-phosphate (ManNAc-6-P), N-acetylneuraminic-acid-9-phosphate (Neu5Ac-9-P), N-acetylneuraminic acid (Neu5Ac), cytidine-5′-monophospho-N-acetylneuraminic acid (CMP-Neu5Ac), acylated D-mannose, or a combination thereof.

(11) In embodiment (11) is presented the method of any one of embodiments (1)-(10), wherein the sugar or a derivative thereof is D-mannose.

(12) In embodiment (12) is presented the method of any one of embodiments (1)-(10), wherein the sugar or a derivative thereof is mannose-6-phosphate (Man-6-P), fructose-6-phosphate (Fruc-6-P), uridine 5′-diphospho-N-acetylglucosamine (UDP-GlcNAc), mannose-1-phosphate (Man-1-P), guanosine diphosphate mannose (GDP-Man), N-acetylglucosamine (GlcNAc), N-acetylgalactosamine (GalNAc), uridine 5′-diphospho-N-acetylgalactosamine (UDP-GalNAc), N-acetyl-D-mannosamine (ManNAc), N-acetyl-D-mannosamine-6-phosphate (ManNAc-6-P), N-acetylneuraminic-acid-9-phosphate (Neu5Ac-9-P), N-acetylneuraminic acid (Neu5Ac), cytidine-5′-monophospho-N-acetylneuraminic acid (CMP-Neu5Ac), or a combination thereof.

(13) In embodiment (13) is presented the method of any one of embodiments (1)-(10), wherein the sugar or a derivative thereof is an acylated D-mannose is of the formula:

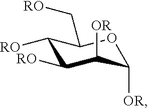

wherein each R independently is hydrogen or

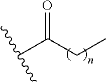

and n is an integer from 0 to 17, and at least one R is

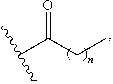

such as, for example, 1-O-acetyl-D-mannopyranose, 2-O-acetyl-D-mannopyranose, 3-O-acetyl-D-mannopyranose, 4-O-acetyl-D-mannopyranose, 6-O-acetyl-D-mannopyranose, 1,2-di-O-acetyl-D-mannopyranose, 1,3-di acetyl-D-mannopyranose, 1,4-di-O-acetyl-D-mannopyranose, 1,6-di-O-acetyl-D-mannopyranose, 2,3-di-O-acetyl-D-mannopyranose, 2,4-di-O-acetyl-D-mannopyranose, 2,6-di-O-acetyl-D-mannopyranose, 3,4-di-O-acetyl-D-mannopyranose, 3,6-di-O-acetyl-D-mannopyranose, 4,6-di-O-acetyl-D-mannopyranose, 1,2,3-tri-O-acetyl-D-mannopyranose, 1,2,4-tri-O-acetyl-D-mannopyranose, 1,2,6-tri-O-acetyl-D-mannopyranose, 1,3,4-tri acetyl-D-mannopyranose, 1,3,6-tri-O-acetyl-D-mannopyranose, 1,4,6-tri-O-acetyl-D-mannopyranose, 2,3,4-tri-O-acetyl-D-mannopyranose, 2,3,6-tri-O-acetyl-D-mannopyranose, 2,4,6-tri-O-acetyl-D-mannopyranose, 3,4,6-tri-O-acetyl-D-mannopyranose, 1,2,3,4-tetra-O-acetyl-D-mannopyranose, 1,2,3,6-tetra-O-acetyl-D-mannopyranose, 1,2,4,6-tetra-O-acetyl-D-mannopyranose, 1,3,4,6-tetra-O-acetyl-D-mannopyranose, 2,3,4,6-tetra-O-acetyl-D-mannopyranose, 1,2,3,4,6-penta-O-acetyl-D-mannopyranose, or a combination thereof.

(14) In embodiment (14) is presented the method of any one of embodiments (9)-(13), wherein administering the therapeutically effective amount of the sugar or a derivative thereof achieves a plasma concentration of at least 2 times greater than a plasma concentration of said sugar or said derivative thereof prior to administration.

(15) In embodiment (15) is presented the method of any one of embodiments (9)-(14), wherein administering the therapeutically effective amount of the sugar or a derivative thereof achieves a plasma concentration of at least 5 times greater than a plasma concentration of said sugar or said derivative thereof prior to administration.

(16) In embodiment (16) is presented the method of any one of embodiments (9)-(15), wherein administering the therapeutically effective amount of the sugar or a derivative thereof achieves a plasma concentration of at least 10 times greater than a plasma concentration of said sugar or said derivative thereof prior to administration.

(17) In embodiment (17) is presented the method of any one of embodiments (1)-(16), wherein administering the therapeutically effective amount of the sugar or a derivative thereof upregulates sialylation of a glycan of a virus particle of the coronavirus.

(18) In embodiment (18) is presented the method of any one of embodiments (1)-(17), wherein administering the therapeutically effective amount of the sugar or a derivative thereof upregulates sialylation of a glycan of a spike protein, a membrane glycoprotein, an envelope protein, a nucleocapsid protein, a phospholipid, or a combination thereof of the virus particle of the coronavirus.

(19) In embodiment (19) is presented the method of embodiment (17) or embodiment (18), wherein the glycan is an N-linked glycan selected from high-mannose N-glycans, complex N-glycans, hybrid N-glycans, or a combination thereof.

(20) In embodiment (20) is presented the method of any one of embodiments (17)-(19), wherein the glycan is an O-linked glycan selected from O-GalNAc O-glycans, O-GlcNAc O-glycans, O-Mannose O-glycans, O-Galactose O-glycans, O-Fucose O-glycans, O-Glucose O-glycans, or a combination thereof.

(21) In embodiment (21) is presented the method of any one of embodiments (17)-(20), wherein the glycan is a glycosphingolipid glycan selected from cerebrosides, gangliosides, globosides, or a combination thereof.

(22) In embodiment (22) is presented the method of any one of embodiments (17)-(21), wherein sialylation of the virus particle of the coronavirus is upregulated by at least 4% relative to a virus particle of the coronavirus in the subject prior to being treated with the sugar or the derivative thereof.

(23) In embodiment (23) is presented the method of any one of embodiments (17)-(22), wherein sialylation of the virus particle of the coronavirus is upregulated by at least 8% relative to a virus particle of the coronavirus in the subject prior to being treated with the sugar or the derivative thereof.

(24) In embodiment (24) is presented the method of any one of embodiments (17)-(23), wherein sialylation of the virus particle of the coronavirus is upregulated by at least 12% relative to a virus particle of the coronavirus in the subject prior to being treated with the sugar or the derivative thereof.

(25) In embodiment (25) is presented the method of any one of embodiments (1)-(24), wherein administering the therapeutically effective amount of the sugar or a derivative thereof downregulates N-glycosylation of a virus particle of the coronavirus.

(26) In embodiment (26) is presented the method of any one of embodiments (1)-(24), wherein administering the therapeutically effective amount of the sugar or a derivative thereof downregulates N-glycosylation of a spike protein, a membrane glycoprotein, an envelope protein, a nucleocapsid protein, a phospholipid, or a combination thereof of the virus particle of the coronavirus.

(27) In embodiment (27) is presented the method of embodiment (25) or embodiment (26), wherein administering the therapeutically effective amount of the sugar or a derivative thereof downregulates the prevalence of an N-linked glycan selected from high-mannose N-glycans, complex N-glycans, hybrid N-glycans, and a combination thereof.

(28) In embodiment (28) is presented the method of any one of embodiments (25)-(27), wherein N-glycosylation of the virus particle of the coronavirus is downregulated by at least 4% relative to a virus particle of the coronavirus in the subject prior to being treated with the sugar or the derivative thereof.

(29) In embodiment (29) is presented the method of any one of embodiments (25)-(28), wherein N-glycosylation of the virus particle of the coronavirus is downregulated by at least 8% relative to a virus particle of the coronavirus in the subject prior to being treated with the sugar or the derivative thereof.

(30) In embodiment (30) is presented the method of any one of embodiments (25)-(29), wherein N-glycosylation of the virus particle of the coronavirus is downregulated by at least 12% relative to a virus particle of the coronavirus in the subject prior to being treated with the sugar or the derivative thereof.

(31) In embodiment (31) is presented the method of any one of embodiments (1)-(30), wherein administering the therapeutically effective amount of the sugar or a derivative thereof downregulates O-glycosylation of a virus particle of the coronavirus.

(32) In embodiment (32) is presented the method of any one of embodiments (1)-(30), wherein administering the therapeutically effective amount of the sugar or a derivative thereof downregulates O-glycosylation of a spike protein, a membrane glycoprotein, an envelope protein, a nucleocapsid protein, a phospholipid, or a combination thereof of the virus particle of the coronavirus.

(33) In embodiment (33) is presented the method of embodiment (31) or embodiment (32), wherein administering the therapeutically effective amount of the sugar or a derivative thereof downregulates the prevalence of an O-linked glycan selected from Tn antigen, STn antigen, core 1 type of O-glycans, core 2 type of O-glycans, core 3 type of O-glycans, core 4 type of O-glycans, core 5 type of O-glycans, core 6 type of O-glycans, core 7 type of O-glycans, and a combination thereof.

(34) In embodiment (34) is presented the method of any one of embodiments (31)-(33), wherein O-glycosylation of the virus particle of the coronavirus is downregulated by at least 4% relative to a virus particle of the coronavirus in the subject prior to being treated with the sugar or the derivative thereof.

(35) In embodiment (35) is presented the method of any one of embodiments (31)-(34), wherein O-glycosylation of the virus particle of the coronavirus is downregulated by at least 8% relative to a virus particle of the coronavirus in the subject prior to being treated with the sugar or the derivative thereof.

(36) In embodiment (36) is presented the method of any one of embodiments (31)-(35), wherein O-glycosylation of the virus particle of the coronavirus is downregulated by at least 12% relative to a virus particle of the coronavirus in the subject prior to being treated with the sugar or the derivative thereof.

(37) In embodiment (37) is presented a method of ameliorating and/or preventing a coronavirus infection in a subject, the method comprising upregulating sialylation of a glycan of a virus particle of the coronavirus.

(38) In embodiment (38) is presented the method of embodiment (37), wherein the method comprises upregulating sialylation of a glycan of a spike protein, a membrane glycoprotein, an envelope protein, a nucleocapsid protein, a phospholipid, or a combination thereof of the virus particle of the coronavirus.

(39) In embodiment (39) is presented the method of embodiment (37) or embodiment (26), wherein the glycan is an N-linked glycan selected from high-mannose N-glycans, complex N-glycans, hybrid N-glycans, or a combination thereof.

(40) In embodiment (40) is presented the method of any one of embodiments (37)-(39), wherein the glycan is an O-linked glycan selected from O-GalNAc O-glycans, O-GlcNAc O-glycans, O-Mannose O-glycans, O-Galactose O-glycans, O-Fucose O-glycans, O-Glucose O-glycans, or a combination thereof.

(41) In embodiment (41) is presented the method of any one of embodiments (37)-(40), wherein the glycan is a glycosphingolipid glycan selected from cerebrosides, gangliosides, globosides, or a combination thereof.

(42) In embodiment (42) is presented a method of ameliorating and/or preventing a coronavirus infection in a subject, the method comprising downregulating N-glycosylation of a virus particle of the coronavirus.

(43) In embodiment (43) is presented the method of embodiment (42), wherein the method comprises downregulating N-glycosylation of a spike protein, a membrane glycoprotein, an envelope protein, a nucleocapsid protein, a phospholipid, or a combination thereof of the virus particle of the coronavirus.

(44) In embodiment (44) is presented the method of embodiment (42) or embodiment (43), wherein the method downregulates the prevalence of an N-linked glycan selected from high-mannose N-glycans, complex N-glycans, hybrid N-glycans, and a combination thereof.

(45) In embodiment (45) is presented a method of ameliorating and/or preventing a coronavirus infection in a subject, the method comprising downregulating O-glycosylation of a virus particle of the coronavirus.

(46) In embodiment (46) is presented the method of embodiment (45), wherein the method comprises downregulating O-glycosylation of a spike protein, a membrane glycoprotein, an envelope protein, a nucleocapsid protein, a phospholipid, or a combination thereof of the virus particle of the coronavirus.

(47) In embodiment (47) is presented the method of embodiment (45) or embodiment (46), wherein the method downregulates the prevalence of an O-linked glycan selected from Tn antigen, STn antigen, core 1 type of O-glycans, core 2 type of O-glycans, core 3 type of O-glycans, core 4 type of O-glycans, core 5 type of O-glycans, core 6 type of O-glycans, core 7 type of O-glycans, and a combination thereof.

(48) In embodiment (48) is presented the method of any one of embodiments (37)-(47), wherein the method comprises administering a therapeutically effective amount of a sugar or a derivative thereof to the subject, wherein the sugar or a derivative thereof is D-mannose, mannose-6-phosphate (Man-6-P), fructose-6-phosphate (Fruc-6-P), uridine 5′-diphospho-N-acetylglucosamine (UDP-GlcNAc), mannose-1-phosphate (Man-1-P), guanosine diphosphate mannose (GDP-Man), N-acetylglucosamine (GlcNAc), N-acetylgalactosamine (GalNAc), uridine 5′-diphospho-N-acetylgalactosamine (UDP-GalNAc), N-acetyl-D-mannosamine (ManNAc), N-acetyl-D-mannosamine-6-phosphate (ManNAc-6-P), N-acetylneuraminic-acid-9-phosphate (Neu5Ac-9-P), N-acetylneuraminic acid (Neu5Ac), cytidine-5′-monophospho-N-acetylneuraminic acid (CMP-Neu5Ac), acylated D-mannose, a metabolite thereof, a prodrug thereof, or a combination thereof.

(49) In embodiment (49) is presented the method of embodiment (48), wherein the sugar or a derivative thereof is administered orally, rectally, transmucosally, intestinally, parenterally, intramuscularly, subcutaneously, intradermally, intramedullaryly, intrathecally, intraventricularly, intravenously, intraperitoneally, intranasally, intraocularly, inhalationally, insufflationally, topically, cutaneously, transdermally, intra-arterially, or a combination thereof.

(50) In embodiment (50) is presented the method of embodiment (48) or embodiment (49), wherein the sugar or a derivative thereof is administered orally.

(51) In embodiment (51) is presented the method of embodiment (48) or embodiment (49), wherein the sugar or a derivative thereof is administered intravenously.

(52) In embodiment (52) is presented the method of any one of embodiments (48)-(51), wherein the sugar or a derivative thereof is D-mannose, mannose-6-phosphate (Man P), fructose-6-phosphate (Fruc-6-P), uridine 5′-diphospho-N-acetylglucosamine (UDP-GlcNAc), mannose-1-phosphate (Man-1-P), guanosine diphosphate mannose (GDP-Man), N-acetylglucosamine (GlcNAc), N-acetylgalactosamine (GalNAc), uridine 5′-diphospho-N-acetylgalactosamine (UDP-GalNAc), N-acetyl-D-mannosamine (ManNAc), N-acetyl-D-mannosamine-6-phosphate (ManNAc-6-P), N-acetylneuraminic-acid-9-phosphate (Neu5Ac-9-P), N-acetylneuraminic acid (Neu5Ac), cytidine-5′-monophospho-N-acetylneuraminic acid (CMP-Neu5Ac), acylated D-mannose, or a combination thereof.

(53) In embodiment (53) is presented the method of any one of embodiments (48)-(51), wherein the sugar or a derivative thereof is D-mannose.

(54) In embodiment (54) is presented the method of any one of embodiments (48)-(51), wherein the sugar or a derivative thereof is mannose-6-phosphate (Man-6-P), fructose-6-phosphate (Fruc-6-P), uridine 5′-diphospho-N-acetylglucosamine (UDP-GlcNAc), N-acetyl-D-mannosamine (ManNAc), mannose-1-phosphate (Man-1-P), guanosine diphosphate mannose (GDP-Man), N-acetylglucosamine (GlcNAc), N-acetylgalactosamine (GalNAc), uridine 5′-diphospho-N-acetylgalactosamine (UDP-GalNAc), N-acetyl-D-mannosamine-6-phosphate (ManNAc-6-P), N-acetylneuraminic-acid-9-phosphate (Neu5Ac-9-P), N-acetylneuraminic acid (Neu5Ac), cytidine-5′-monophospho-N-acetylneuraminic acid (CMP-Neu5Ac), or a combination thereof.

(55) In embodiment (55) is presented the method of any one of embodiments (48)-(51), wherein the sugar or a derivative thereof is an acylated D-mannose is of the formula:

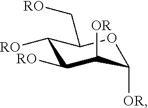

wherein each R independently is hydrogen or

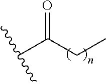

and n is an integer from 0 to 17, and at least one R is

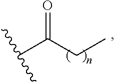

such as, for example, 1-O-acetyl-D-mannopyranose, 2-O-acetyl-D-mannopyranose, 3-O-acetyl-D-mannopyranose, 4-O-acetyl-D-mannopyranose, 6-O-acetyl-D-mannopyranose, 1,2-di-O-acetyl-D-mannopyranose, 1,3-di-O-acetyl-D-mannopyranose, 1,4-di-O-acetyl-D-mannopyranose, 1,6-di-O-acetyl-D-mannopyranose, 2,3-di-O-acetyl-D-mannopyranose, 2,4-di-O-acetyl-D-mannopyranose, 2,6-di-O-acetyl-D-mannopyranose, 3,4-di-O-acetyl-D-mannopyranose, 3,6-di-O-acetyl-D-mannopyranose, 4,6-di-O-acetyl-D-mannopyranose, 1,2,3-tri-O-acetyl-D-mannopyranose, 1,2,4-tri-O-acetyl-D-mannopyranose, 1,2,6-tri-O-acetyl-D-mannopyranose, 1,3,4-tri-O-acetyl-D-mannopyranose, 1,3,6-tri-O-acetyl-D-mannopyranose, 1,4,6-tri-O-acetyl-D-mannopyranose, 2,3,4-tri-O-acetyl-D-mannopyranose, 2,3,6-tri-O-acetyl-D-mannopyranose, 2,4,6-tri-O-acetyl-D-mannopyranose, 3,4,6-tri-O-acetyl-D-mannopyranose, 1,2,3,4-tetra-O-acetyl-D-mannopyranose, 1,2,3,6-tetra-O-acetyl-D-mannopyranose, 1,2,4,6-tetra-O-acetyl-D-mannopyranose, 1,3,4,6-tetra-O-acetyl-D-mannopyranose, 2,3,4,6-tetra-O-acetyl-D-mannopyranose, 1,2,3,4,6-penta-O-acetyl-D-mannopyranose, or a combination thereof.

(56) In embodiment (56) is presented the method of any one of embodiments (52)-(55), wherein administering the therapeutically effective amount of the sugar or a derivative thereof achieves a plasma concentration of at least 2 times greater than a plasma concentration of said sugar or said derivative thereof prior to administration.

(57) In embodiment (57) is presented the method of any one of embodiments (52)-(55), wherein administering the therapeutically effective amount of the sugar or a derivative thereof achieves a plasma concentration of at least 5 times greater than a plasma concentration of said sugar or said derivative thereof prior to administration.

(58) In embodiment (58) is presented the method of any one of embodiments (52)-(55), wherein administering the therapeutically effective amount of the sugar or a derivative thereof achieves a plasma concentration of at least 10 times greater than a plasma concentration of said sugar or said derivative thereof prior to administration.

(59) In embodiment (59) is presented the method of any one of embodiments (37)-(58), wherein sialylation of the virus particle of the coronavirus is upregulated by at least 4% relative to a virus particle of the coronavirus in the subject prior to being treated with the sugar or the derivative thereof.

(60) In embodiment (60) is presented the method of any one of embodiments (37)-(58), wherein sialylation of the virus particle of the coronavirus is upregulated by at least 8% relative to a virus particle of the coronavirus in the subject prior to being treated with the sugar or the derivative thereof.

(61) In embodiment (61) is presented the method of any one of embodiments (37)-(58), wherein sialylation of the virus particle of the coronavirus is upregulated by at least 12% relative to a virus particle of the coronavirus in the subject prior to being treated with the sugar or the derivative thereof.

(62) In embodiment (62) is presented the method of any one of embodiments (37)-(61), wherein N-glycosylation of the virus particle of the coronavirus is downregulated by at least 4% relative to a virus particle of the coronavirus in the subject prior to being treated with the sugar or the derivative thereof.

(63) In embodiment (63) is presented the method of any one of embodiments (37)-(61), wherein N-glycosylation of the virus particle of the coronavirus is downregulated by at least 8% relative to a virus particle of the coronavirus in the subject prior to being treated with the sugar or the derivative thereof.

(64) In embodiment (64) is presented the method of any one of embodiments (37)-(61), wherein N-glycosylation of the virus particle of the coronavirus is downregulated by at least 12% relative to a virus particle of the coronavirus in the subject prior to being treated with the sugar or the derivative thereof.

(65) In embodiment (65) is presented the method of any one of embodiments (37)-(64), wherein O-glycosylation of the virus particle of the coronavirus is downregulated by at least 4% relative to a virus particle of the coronavirus in the subject prior to being treated with the sugar or the derivative thereof.

(66) In embodiment (66) is presented the method of any one of embodiments (37)-(64), wherein O-glycosylation of the virus particle of the coronavirus is downregulated by at least 8% relative to a virus particle of the coronavirus in the subject prior to being treated with the sugar or the derivative thereof.

(67) In embodiment (67) is presented the method of any one of embodiments (37)-(64), wherein O-glycosylation of the virus particle of the coronavirus is downregulated by at least 12% relative to a virus particle of the coronavirus in the subject prior to being treated with the sugar or the derivative thereof.

(68) In embodiment (68) is presented the method of any one of embodiments (37)-(67), wherein the coronavirus infection is a severe acute respiratory syndrome coronavirus 2 (SARS-CoV-2) related infection.

(69) In embodiment (69) is presented the method of embodiment (68), wherein the severe acute respiratory syndrome coronavirus 2 (SARS-CoV-2) related infection is COVID-19.

(70) In embodiment (70) is presented the method of any one of embodiments (37)-(67), wherein the coronavirus infection is a severe acute respiratory syndrome coronavirus (SARS-CoV) related infection.

(71) In embodiment (71) is presented the method of any one of embodiments (37)-(67), wherein the coronavirus infection is a Middle East respiratory syndrome (MERS) related infection.

### EXAMPLES

The following examples further illustrate the invention but, of course, should not be construed as in any way limiting its scope.

### Example 1

This example demonstrates the beneficial cytoprotection and reduction in SARS-CoV-2 virus replication exhibited by the treatment with D-mannose in cultured cells.

Human lung epithelial carcinoma cell line (A549) was genetically engineered to overexpress angiotensin-converting enzyme 2 (ACE2), which is the primary entry receptor of SARS-CoV-2. Expression of ACE2 was validated by staining cells with rabbit anti-human-ACE2 antibody (10108-RP01, commercially available from Sino Biological Inc.) and anti-rabbit IgG-FITC antibody (Invitrogen F2765, commercially available from Thermo Fisher Scientific). Stained cells were analyzed by CytoFlex flow cytometer, commercially available from Beckman Coulter Diagnostics. Validated cells (A549 ACE2+) were cultured in the DMEM (Dulbecco's Modified Eagle's Medium with 4.5 g/L glucose, L-glutamine, and sodium pyruvate, 10% fetal bovine serum, 1% streptomycin and penicillin) supplemented with or without 100 mM D-mannose (M8574, commercially available from Sigma) for 10 days. During the 10-day period of time, cells were split 3 times and 25% of the cell population was kept for continuous culturing under the same conditions. After 10 days, cells were seeded into 96-well cell culture plates as a density of 8×10³ cells/well for SARS-CoV-2 infection.

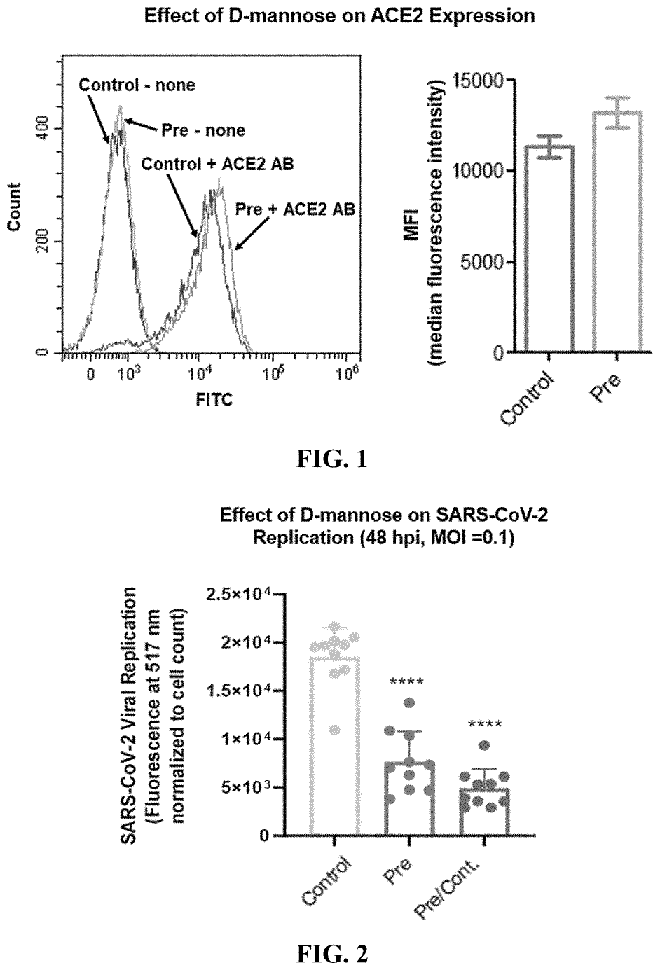

Before infection, ACE2 expression was analyzed for both D-mannose treated and non-treated cells to determine whether D-mannose treatment alters ACE2 expression. The same cells for the infection experiment were analyzed by flow cytometer. 3×10⁵ cells, either treated or non-treated, were stained by rabbit anti-human ACE2 antibody (10108-RP01, used at a concentration of 2.5 μg/mL in 1×PBS) for 1 hour at 4° C., and after incubation, were washed and a secondary goat anti-rabbit IgG (H+L) antibody—FITC (F2765, used as 1:200 in 1×PBS) was used to stain the cells for 1 hour at 4° C., and after incubation, were washed and analyzed by CytoFlex flow cytometer. The results are set forth in FIG. 1. As demonstrated by FIG. 1, D-mannose treatment did not significantly change ACE2 expression, although a negligible increase in ACE2 expression was observed.

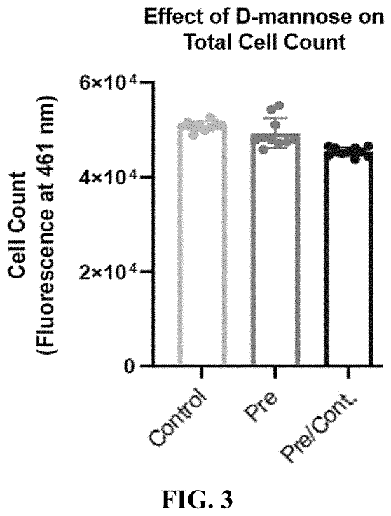

The D-mannose treated cells were divided into two groups. One group continuously received D-mannose (100 mM) in culture medium (2% fetal bovine serum), and for the other group, D-mannose was withdrawn from the culture medium (2% fetal bovine serum). Reporter mNeonGreen SARS-CoV-2 viruses were added to the cells with a multiplicity of infection (MOI) value=0.1. Reporter mNeonGreen SARS-CoV-2 virus was constructed from the virus strain (2019-nCoV/USA_WA1/2020) isolated from the first reported SARS-CoV-2 case in the US. Virus stocks were amplified in Vero E6 cells to Passage 1 (P1) with a titer of 9.7×10⁵ PFU/mL. At 48 hours post-infection, cells were imaged by the Celigo Imaging Cytometer, and SARS-CoV-2 replication was quantified using the mNeonGreen reporter fluorescence. Reporter expressions were normalized to total cell count quantified by Hoechst nuclear stain of total cells and then compared among treatment groups. Total cell counts were analyzed from uninfected culture to determine cytotoxicity. Compared with untreated cells, both groups of D-mannose-treated cells presented significant cytoprotection 48 hours post-infection, as evidenced by FIG. 2. FIG. 2 further shows that the group with continuous D-mannose treatment showed an almost 70% reduction in viral replication. In addition, the total count was measured to determine the effect on cell growth and cytotoxicity exhibited by D-mannose, and the results are set forth in FIG. 3. As demonstrated by FIG. 3, cells treated with D-mannose had a negligible difference in cell count. Thus, D-mannose provides antiviral activity against SARS-CoV-2 viruses with negligible growth inhibition and no observed cytotoxicity, as demonstrated by FIG. 2 and FIG. 3.

Data of flow cytometry was analyzed using CytExpert and Kaluza, both of which are commercially available from Beckman Coulter Diagnostics. Data from the infection experiment was analyzed using GraphPad Prism V9.1.2, and an Analysis of Variance with Multiple Comparisons test.

### Example 2

This example demonstrates the glycan-binding specificities of various lectins used to determine the degree of N-linked glycosylation, O-linked glycosylation, or sialylation of the coronavirus particles, including, for example, the spike (S) protein, an envelope (E) protein, a membrane (M) protein, or a nucleocapsid protein of the virus particle of the coronavirus.

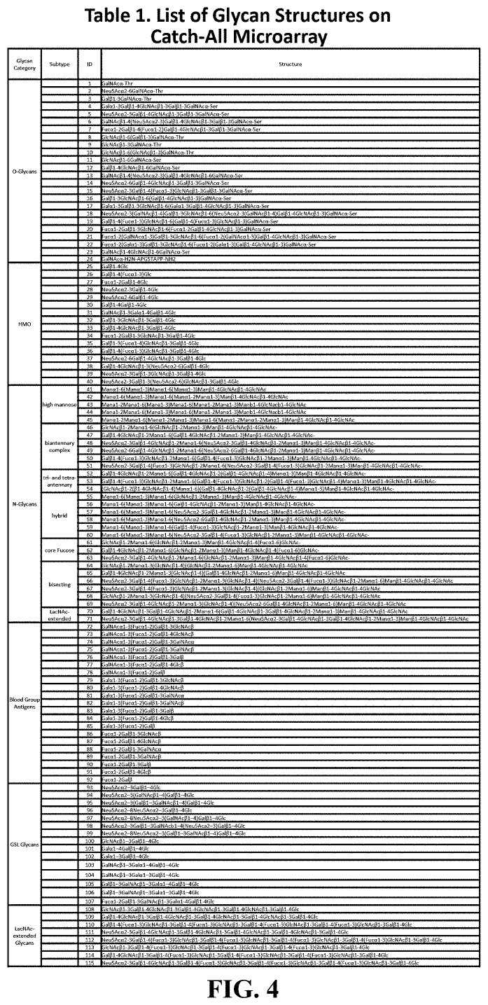

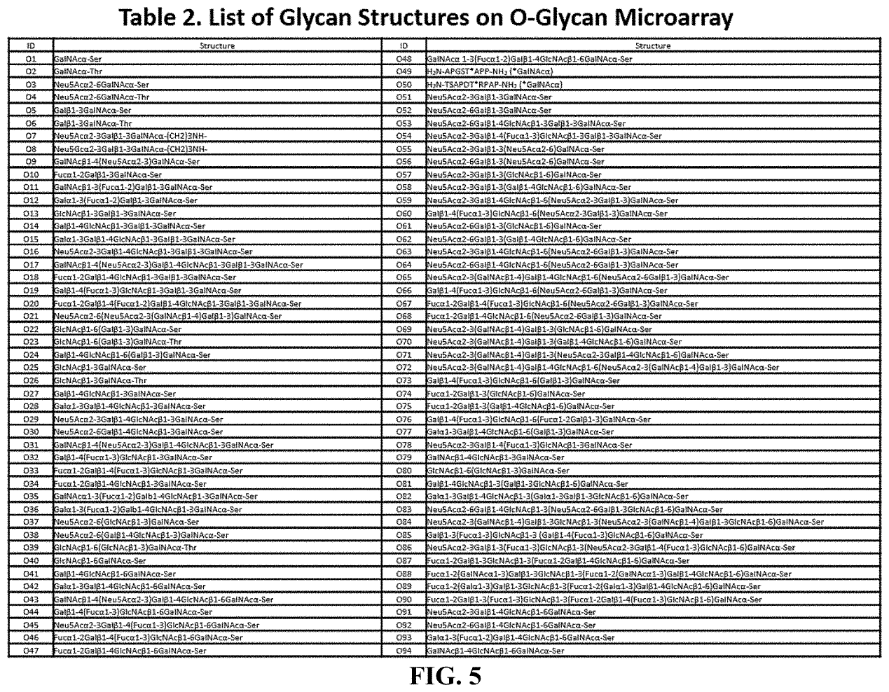

To generate Catch-All microarrays to determine the specificities of lectins, 115 diversified amino-tagged glycans were immobilized on a multivalent N-hydroxysuccinimide (NHS) coated glass slide by a microarray spotter, as described in Zhang (U.S. Patent Application Publication 2019/0128881). An O-glycan microarray was purchased from ZBiotech. As demonstrated by FIG. 4, the glycans of the Catch-All microarray represent diversified structures of O-glycans, N-glycans, human milk oligosaccharides (HMO), blood group antigens (BGA), Glycosphingolipid (GSL) glycans, and LacNAc—extended glycans. The structures of O-glycan microarrays are listed in FIG. 5.

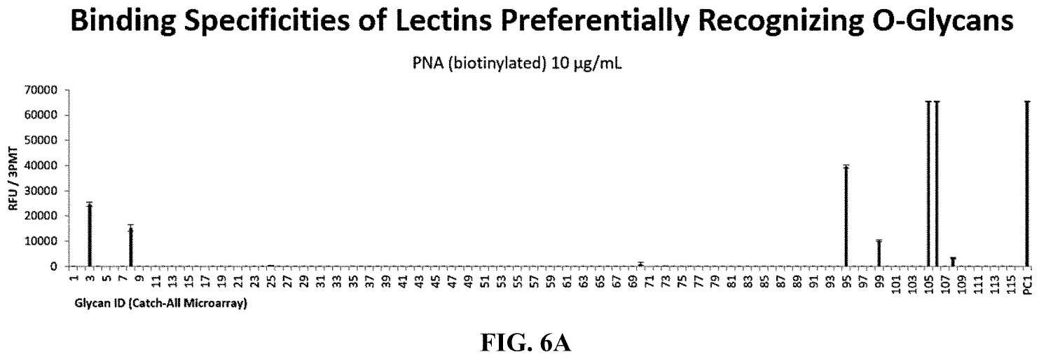

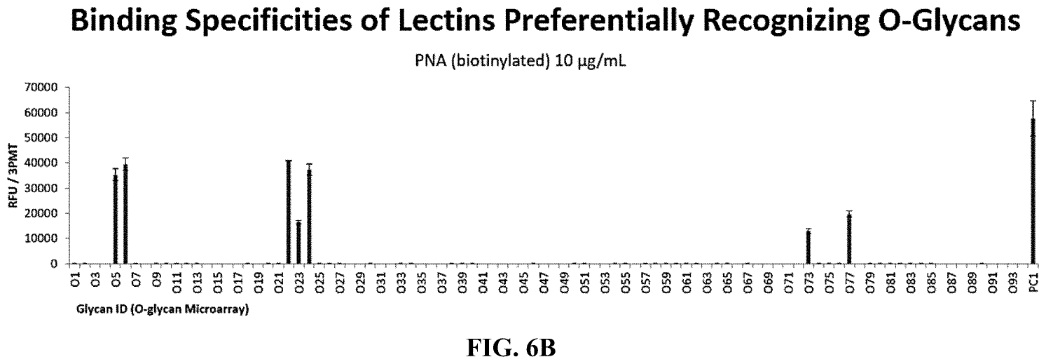

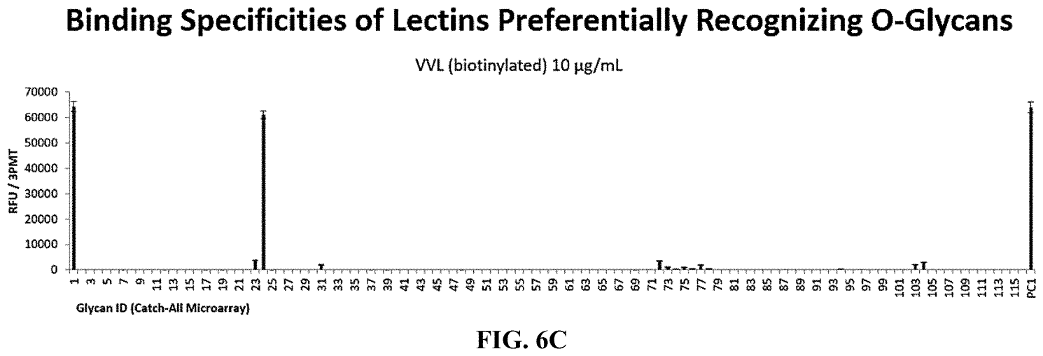

Specifically, biotinylated lectins were dissolved in TBS-T buffer (supplemented with 2 mM of CaCl₂) and MgCl₂) to make 10 μg/mL assay solutions. To determine the glycan-binding specificities, lectin assay solutions were incubated with Catch-All or O-glycan microarray slides for 1 hour at room temperature. After incubation, microarray slides were washed with TBS-T buffer and incubated with Streptavidin-Cy3 (Product #434315, commercially available from Thermo Fisher Scientific, and used as 2 μg/mL in TBS-T buffer supplemented with 2 mM of CaCl₂) and MgCl₂) for 1 hour at room temperature. Slides were then washed with TBS-T buffer and ultrapure water and scanned by a microarray slide scanner (InnoScan 710, commercially available from Innopsys) to obtain fluorescence signals. The results are set forth in FIGS. 6A-6C, FIGS. 7A-7D, FIGS. 8A and 8B, and FIGS. 9A-9D.

Lectins, which were assessed for their glycan-binding specificities, include: (1) biotinylated Calystegia Sepium (Calsepa) lectin (BA-8011-1, commercially available from EY Labs); (2) biotinylated Erythrina Cristagalli (ECL) lectin (B-1145-5, commercially available from Vector Laboratories); (3) biotinylated Maackia Amurensis Lectin I (MAL1) (B-1315-2, commercially available from Vector Laboratories); (4) biotinylated Maackia Amurensis Lectin II (MAL2) (B-1265-1, commercially available from Vector Laboratories); (5) biotinylated Musa Paradisiaca lectin (BanLec) (B-1415, commercially available from Vector Laboratories); (6) biotinylated Peanut Agglutinin (PNA) lectin (B-1075-5, commercially available from Vector Laboratories); (7) biotinylated Pisum Sativum Agglutinin (PSA) lectin (B-1055-5, commercially available from Vector Laboratories); (8) biotinylated Ralstonia solanacearum lectin (RSL) (L1258, commercially available from GLYcoDiag); (9) biotinylated Sambucus Nigra (SNA) lectin (B-1305-2, commercially available from Vector Laboratories); and (10) biotinylated Vicia Villosa (VVL) lectin (B-1235-2, commercially available from Vector Laboratories).

As demonstrated by FIGS. 6A-6C, PNA and VVL recognize specific O-linked glycans. More particularly, PNA preferentially recognizes T antigen, a galactosyl (β-1,3)N-acetylgalactosamine structure. In addition, PNA also recognizes Core 2 or extended Core 2 structures without terminal sialic acids. VVL preferentially recognizes Tn antigen, an α-N-acetylgalactosamine residue linked to serine or threonine of a protein. In addition, PNA also identifies other α- or β-linked terminal N-acetylgalactosamine.

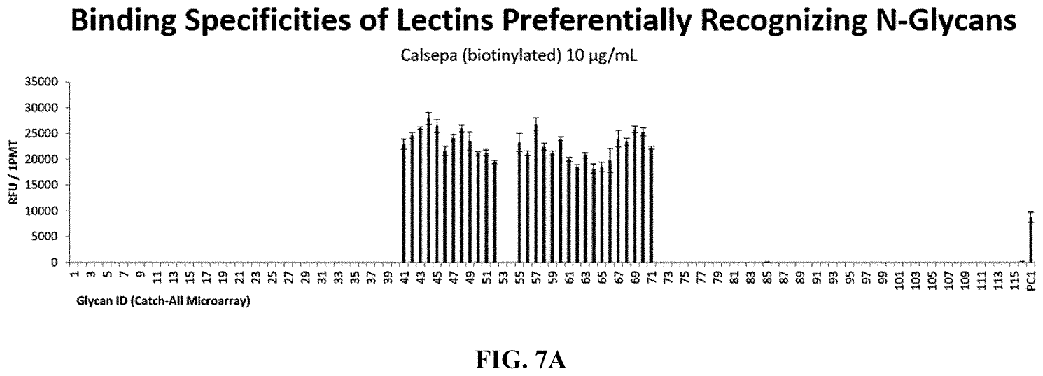

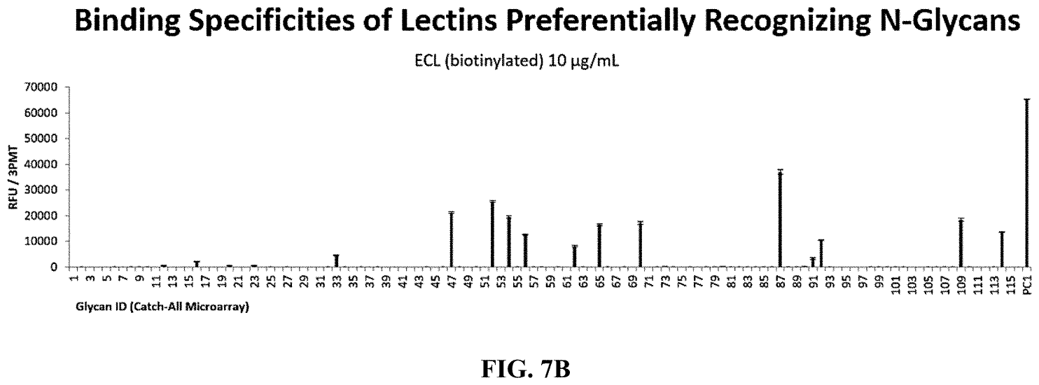

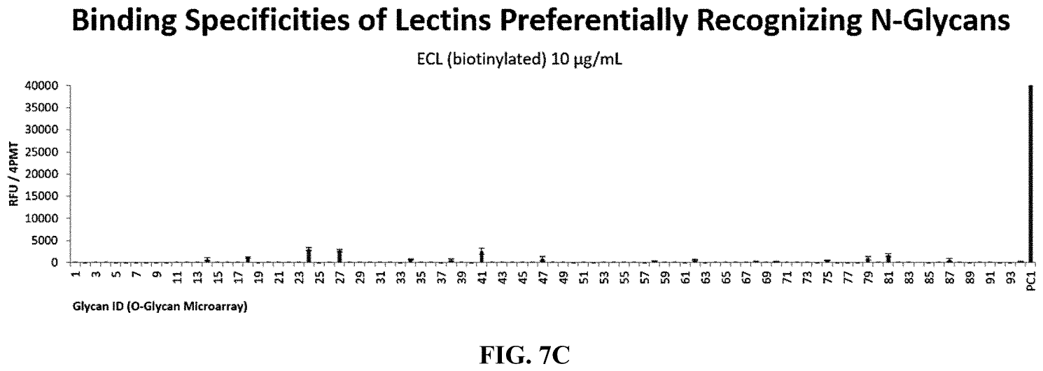

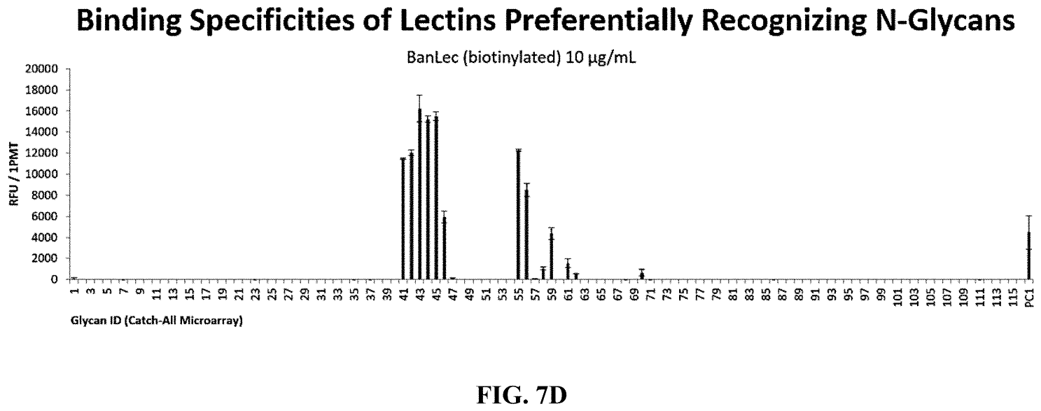

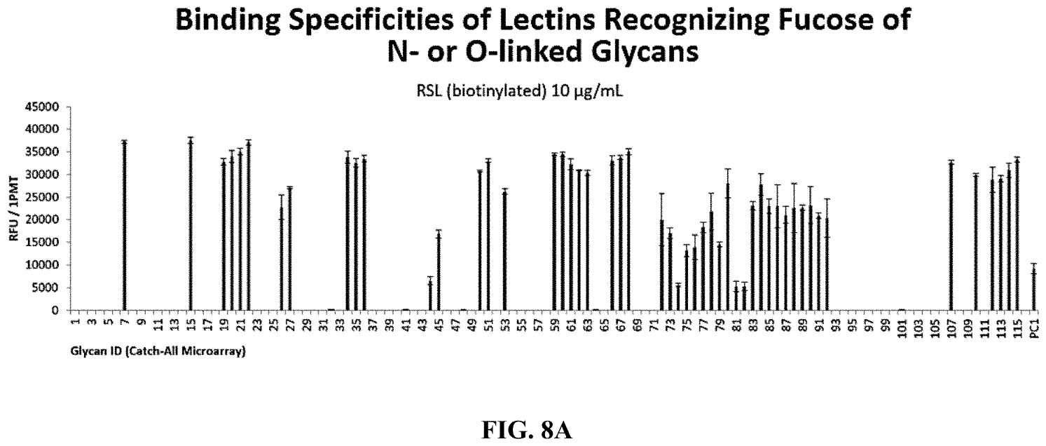

As demonstrated by FIGS. 7A-7D, Calsepa, ECL, and BanLec are N-glycan-binding lectins. More particularly, Calsepa generally binds with all N-linked glycans without differentiating a specific subtype of N-glycans with or without terminal sialic acids. ECL recognizes terminal type 2 LacNAc epitope of an N-glycan or O-glycan. The presence a fucose (Lewis X structure) or a terminal sialic acid on the epitope blocks the ECL binding. BanLec preferentially recognizes high-mannose N-glycans and hybrid N-glycans without terminal sialic acids.

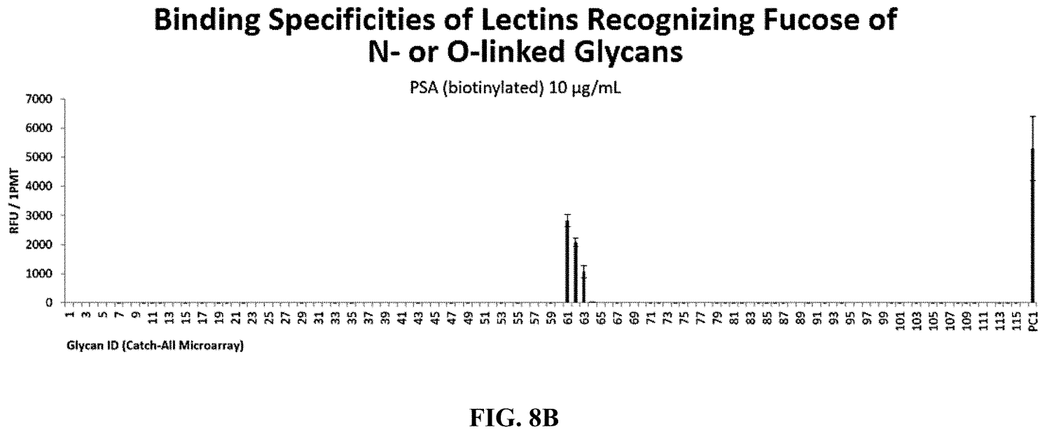

As demonstrated by FIGS. 8A and 8B, RSL and PSA are fucose-binding lectins. More particularly, RSL recognizes all fucose-containing glycans which include fucose-containing N-glycans, O-glycans, and blood group antigens. The presence of terminal sialic acids doesn't significantly affect the binding of RSL to these glycans. In comparison with the broad binding specificities of RSL, PSA only recognizes core fucose of N-glycans with or without terminal sialic acids.

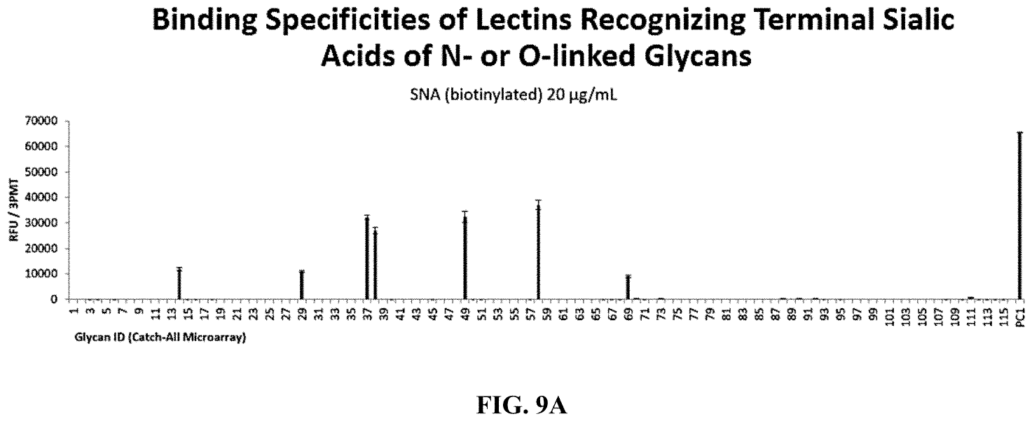

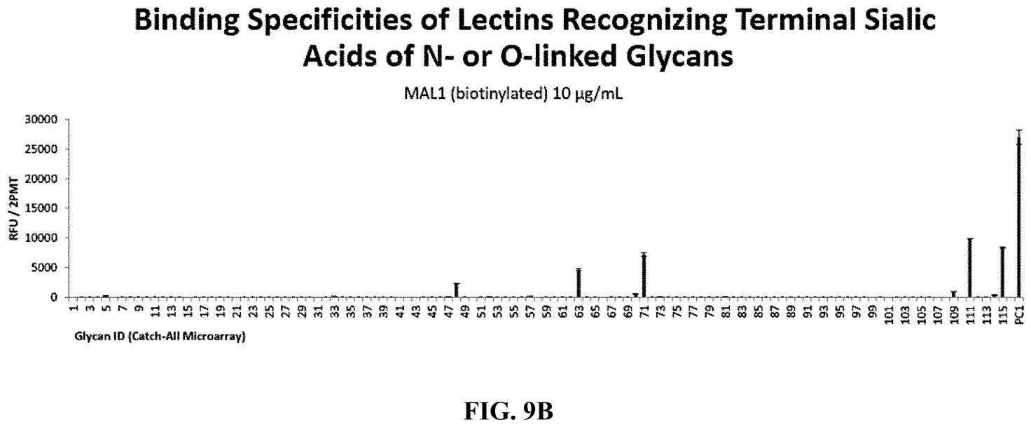

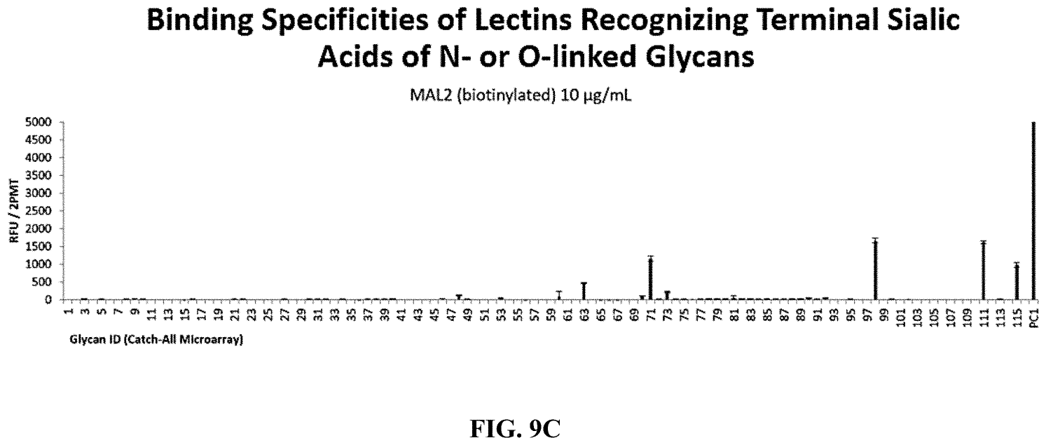

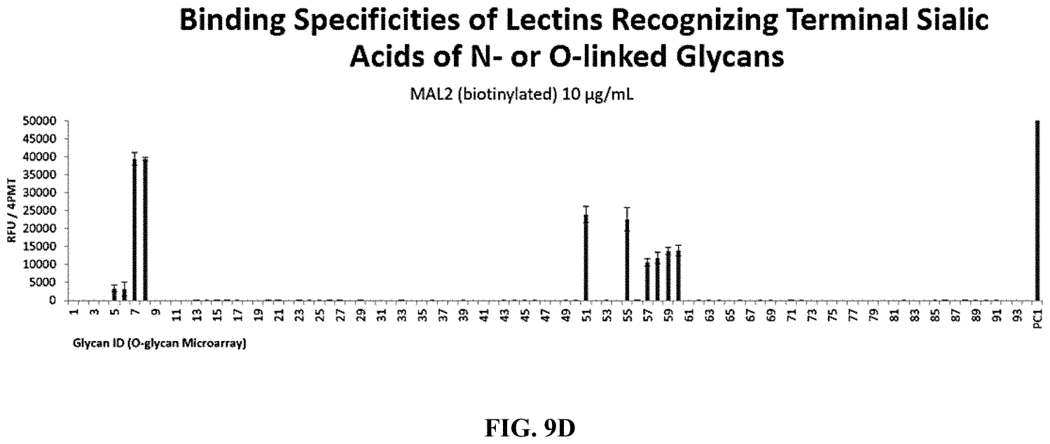

As demonstrated by FIGS. 9A-9D, SNA, MAL1 and MAL2 are sialoglycan-specific lectins. More particularly, SNA recognizes terminal α-2,6 sialic acids (either Neu5Ac or Neu5Gc). In stark contrast, MAL1 and MAL2 preferentially recognize terminal α-2,3 sialic acids (either Neu5Ac or Neu5Gc). In that regard, the presence of fucose (SLeX or Sda antigen) blocks the MAL binding.

### Example 3

This example demonstrates the ability to prevent SARS-CoV-2 S Trimer-ACE2 interaction, exhibited by the presence of sialic acids on the SARS-CoV-2 S protein.

To investigate whether sialic acids of SARS-CoV-2 S protein inhibit ACE2 binding, recombinant SARS-CoV-2 S Trimers were immobilized on a solid surface (glass slide). The recombinant SARS-CoV-2 S protein (SPN-C52H9, commercially available from Acro Biosystems) was expressed in HEK293T cells with T4 fibritin trimerization motif and purified with a polyhistidine (His) tag at the C-terminus. Proline substitutions (F817P, A892P, A899P, A942P, K986P, V987P) and alanine substitutions (R683A and R685A) were introduced to stabilize the trimeric prefusion state of S protein and abolish the furin cleavage site. Recombinant S Trimers (0.2 or 0.4 μg/μL) were immobilized on an NHS-functionalized slide by a microarray spotter, as described in U.S. Patent Application Publication 2019/0128881. A batch of array slides with immobilized S trimers were generated.

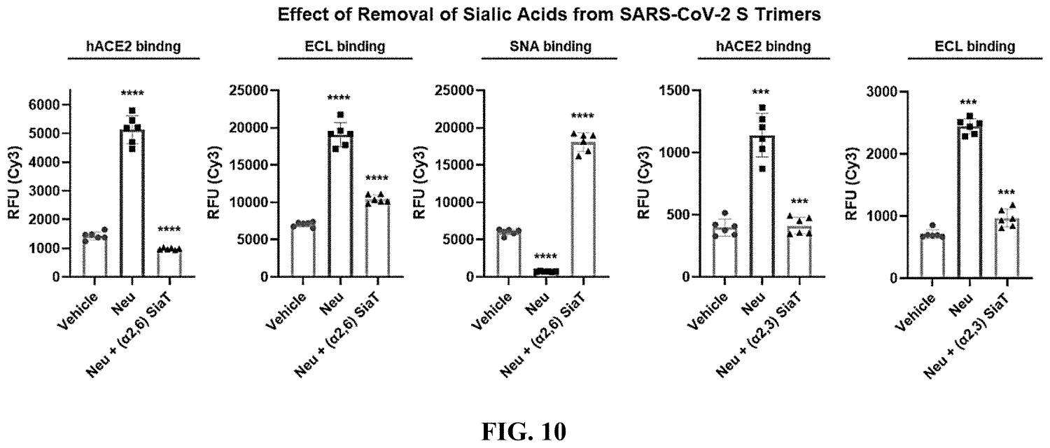

The slide was treated with neuraminidase (11585886001—from Clostridium perfringens, commercially available from Roche, and used as 50 mU/100 μL in 1×PBS) for 8 hours at room temperature and then washed, and incubated with recombinant human ACE2 (0108-H08H, commercially available from Sino Biological Inc., and used as 4 μg/mL in 1× PBS) for 1 hour at room temperature. After incubation, the slide was washed and a secondary rabbit anti-human ACE2 antibody (10108-RP01, commercially available from Sino Biological Inc., and used at a concentration of 2 μg/mL) was added and incubated for an additional 1 hour. After incubation, the slide was washed and a tertiary goat anti-rabbit IgG (H+L) Cy3 antibody (ab6838, commercially available from ABCAM, and used as 4 μg/mL) was added and incubated for 1 hour at room temperature. After incubation, the slide was washed and scanned by a scanner (InnoScan 710, commercially available from Innopsys) to obtain fluorescence signals. The results are set forth in FIG. 10.

As demonstrated by the results set forth in FIG. 10, reduction of sialic acids from SARS-CoV-2 S trimers increased S protein—ACE2 binding by an average of 328%.

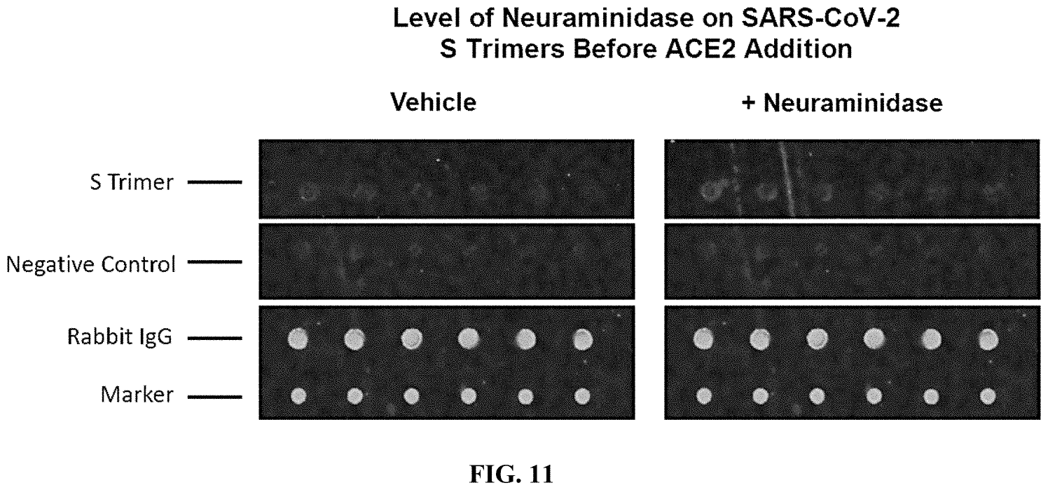

To determine whether neuraminidase treatment left neuraminidase on the S trimers that potentially interferes with S trimer—ACE2 interaction, a neuraminidase—processed slide was incubated with a rabbit anti-neuraminidase antibody (11680-RP01, Clostridium perfringens, commercially available from Sino Biological Inc., and used as 5 μg/mL in TBS-T) for 1 hour at room temperature. After incubation, the slide was washed and incubated with a secondary goat anti-rabbit IgG (H+L) Cy3 antibody (ab6838, commercially available from ABCAM, and used as 4 μg/mL). After incubating with the secondary antibody for 1 hour at room temperature, the slide was washed and scanned to obtain fluorescence signals. The results are set forth in FIG. 11.

As demonstrated by the results set forth in FIG. 11, there was no neuraminidase detected besides a negligible background. Thus, the observed enhancement of binding is a result of removing sialic acids from S trimers.

To further validate the foregoing observation, sialic acids were restored back to the S trimers after the neuraminidase (Clostridium perfringens) treatment, and S trimers with restored sialic acids were evaluated for ACE2 binding. To enzymatically restore the sialic acids back to the S trimers, the neuraminidase—processed slide from above was further incubated with α2,6-sialyltransferase I (ST6GAL1) or α2,3-sialyltransferase III (ST3GAL1) in TBS-T buffer supplemented with 10 mM CMP-Neu5Ac at room temperature overnight. The α2,6-sialyltransferase I was obtained from GlycoExpression Technologies and was used as 15 μL/100 μL of the reaction. The α2,3-sialyltransferase III (ST3GAL1) was obtained from GlycoExpression Technologies and was used as 3 μg/100 μL of the reaction. After incubation and washing, recombinant human ACE2, rabbit anti-human ACE2 antibody, and goat anti-rabbit IgG (H+L) Cy3 antibody were sequentially added and incubated with the slide as described above. The results are set forth in FIG. 10.

As demonstrated by FIG. 10, restoring sialic acids of S trimers reduced S protein—ACE2 binding to the level similar to the binding of native S trimers. This result further indicates that sialic aids of S trimmer protein prevent S trimer—ACE2 binding.

Neuraminidase from Clostridium perfringens cleaves terminal sialic acid residues that are α2,3-, α2,6-, or α2,8-linked to Gal, GlcNAc, GalNAc, AcNeu, GlcNeu, oligosaccharides, glycolipids, or glycoproteins. To validate whether neuraminidase treatment successfully removed sialic acids from S trimers, the neuraminidase—processed slide was incubated with biotinylated—Erythrina Cristagalli (ECL) lectin or Sambucus Nigra (SNA) lectin for 1 hour at room temperature. The biotinylated—ECL (B-1145-5, commercially available from Vector Laboratories) was used as 5 μg/mL in carbo-free buffer supplemented with 2 mM CaCl₂) and MgCl₂. The biotinylated—SNA (B-1305-2, commercially available from Vector Laboratories) was used as 3 μg/mL in carbo-free buffer supplemented with 2 mM CaCl₂) and MgCl₂. After incubation, the slide was washed and Streptavidin—Cy3 (434315, commercially available from Thermo Fisher Scientific, and used as 2 μg/mL in carbo-free buffer supplemented with 2 mM CaCl₂) and MgCl₂) was added and incubated for 1 hour at room temperature. After incubation, the slide was washed and scanned by a scanner (InnoScan 710, commercially available from Innopsys) to obtain fluorescence signals. The results are set forth in FIG. 10.

As demonstrated by FIG. 10, neuraminidase treatment significantly enhanced ECL binding while reducing SNA binding to the S trimers. In that regard, ECL recognizes terminal galactose without sialic acids and SNA binds preferentially to α2,6-linked sialic acids. Therefore, this result verified that sialic acids were removed from S trimers.

To further determine whether sialyltransferase treatment restored the sialic acids to the S trimers, the neuraminidase—processed and then sialyltransferase—incubated slide was analyzed by the same methods described above. The results are also set forth in FIG. 10.

As demonstrated by FIG. 10, the neuraminidase—processed, sialyltransferase treated slides reduced ECL binding, while increasing SNA binding. This result verified that sialyltransferase restored sialic acids back to the S trimers.

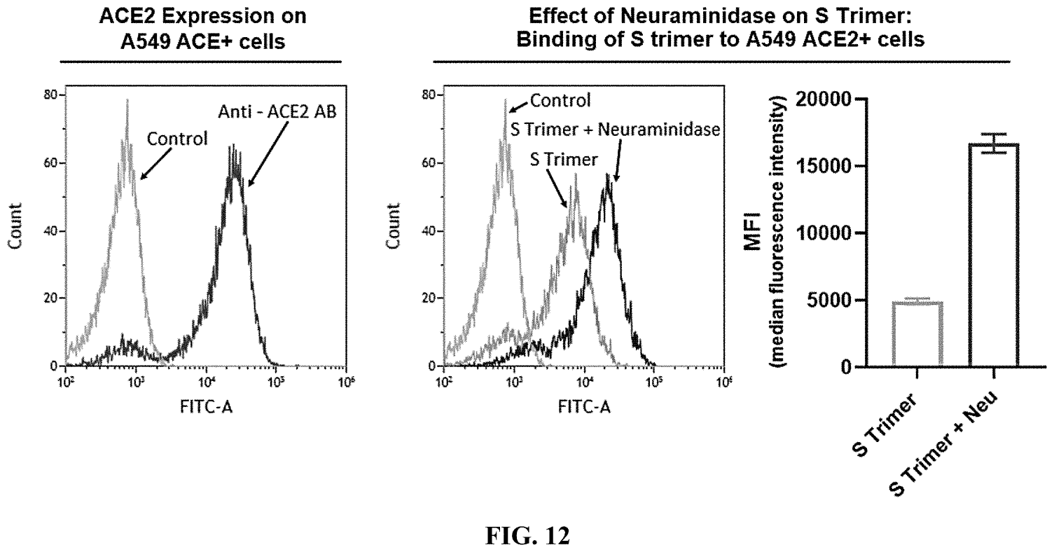

To validate the findings in live cells, cell bindings of native S trimers and S trimers processed with neuraminidase (Clostridium perfringens) were evaluated and compared. S trimers (SPN-C52H9, commercially available from Acro Biosystems, and used as 4 μg/100 μL) were mixed with vehicle (1×PBS) or vehicle containing neuraminidase (11585886001, commercially available from Roche, and used as 50 mU/100 μL in 1×PBS). The mixtures were incubated at room temperature for 12 hours. After incubation, both mixtures, plus a vehicle only control were used to stain A549 ACE2+ cells for flow cytometry analysis. 3×10⁵ A549 ACE2+ cells were incubated with the mixtures or vehicle only control (100 μL) for 1 hour at room temperature. After incubation, the cells were washed and a mouse anti-S protein antibody (GTX632604, 1A9, commercially available from GeneTex, and used as 1:200) was used to stain the cells for 1 hour at 4° C., and after incubation, the cells were washed and a secondary goat anti-mouse IgG (H+L) antibody—FITC (F2761, commercially available from Invitrogen, and used as 8 μg/mL in 1×PBS) was used to stain the cells for 1 hour at 4° C. After incubation, the cells were washed and analyzed by flow cytometer (CytoFLEX, serial #BB51412, commercially available from Beckman Coulter). The results are set forth in FIG. 12.

As demonstrated by FIG. 12, the neuraminidase—processed S trimers present higher affinity for the A549 ACE2+ cells as compared to the native S trimers. Together, these results indicate that the upregulation of sialic acids on SARS-CoV-2 S protein prevents SARS-CoV-2 S protein—ACE2 binding.

Data of flow cytometry was analyzed using CytExpert and Kaluza, both of which are commercially available from Beckman Coulter Diagnostics. Fluorescent signals of array slides were collected using Mapix (Innopsys). Data from the binding experiment was analyzed using GraphPad Prism V9.1.2 and an Analysis of Variance with Multiple Comparisons test.

### Example 4

This example demonstrates the ability to prevent SARS-CoV-2 S Trimer—ACE2 interaction, exhibited by the reduction of N-glycans on the SARS-CoV-2 S protein.

Recombinant SARS-CoV-2 S Trimers and RBD protein were immobilized on the multivalent N-hydroxysuccinimide (NHS) coated glass slide by a microarray spotter. The recombinant SARS-CoV-2 S protein (SPN-C52H9, commercially available from Acro Biosystems) was expressed in HEK293T cells with T4 fibritin trimerization motif and purified with a polyhistidine (His) tag at the C-terminus. Proline substitutions (F817P, A892P, A899P, A942P, K986P, V987P) and alanine substitutions (R683A and R685A) were introduced to stabilize the trimeric prefusion state of S protein and abolish the furin cleavage site. RBD protein was expressed in the HEK293T cells. Recombinant S Trimers and RBD (0.2 or 0.4 μg/μL) were immobilized on an NHS-functionalized slide by a microarray spotter, as described in Zhang (U.S. Patent Application Publication 2019/0128881), and a batch of array slides with immobilized S trimers and RBD were generated.

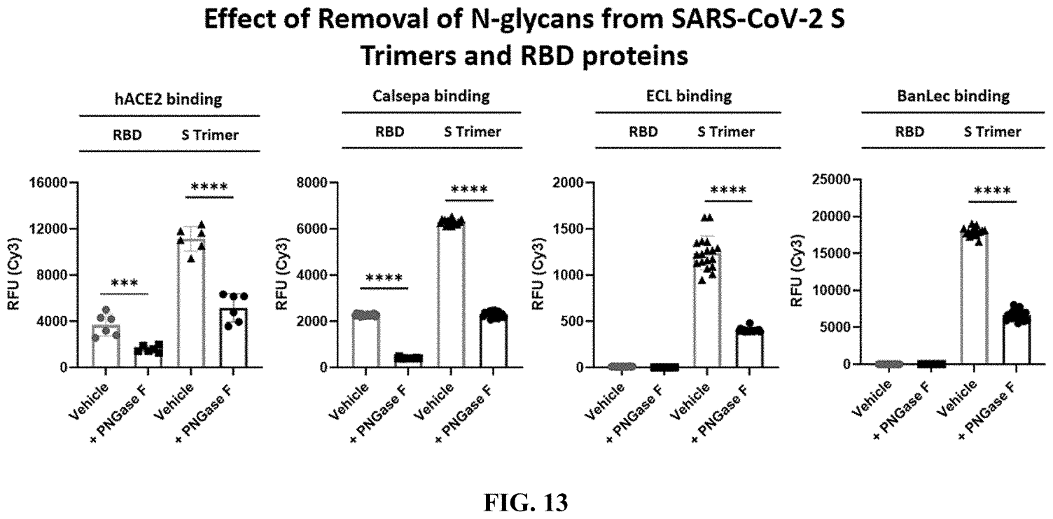

The slides were treated with PNGase F (P0704S, commercially available from NEB, and used as 500 U/50 μL in 1×PBS for a single subarray) for 8 hours at room temperature and then washed, and incubated with recombinant human ACE2 (0108-H08H, commercially available from Sino Biological Inc., and used as 4 μg/mL in 1×PBS) for 1 hour at room temperature. After incubation, the slides were washed and a secondary rabbit anti-human ACE2 antibody (10108-RP01, commercially available from Sino Biological Inc., and used at a concentration of 2 μg/mL) was added and incubated for an additional 1 hour. After incubation, the slides were washed and a tertiary goat anti-rabbit IgG (H+L) Cy3 antibody (ab6838, commercially available from ABCAM, and used as 4 μg/mL) was added and incubated for 1 hour at room temperature. After incubation, the slides were washed and scanned by a scanner (InnoScan 710, commercially available from Innopsys) to obtain fluorescence signals, and the results are set forth in FIG. 13.

As demonstrated by the results set forth in FIG. 13, reduction of N-glycans from SARS-CoV-2 S trimers significantly reduced S protein—ACE2 binding by an average of 48% and RBD-ACE2 binding by an average of 43%.

To validate whether PNGase F treatment successfully removed N-glycans from RBD and S trimers, the PNGase F—processed slides were incubated with biotinylated—Calystegia Sepium (Calsepa) lectin, Erythrina Cristagalli (ECL) lectin or Musa Paradisiaca lectin (BanLec) for 1 hour at room temperature. The biotinylated—Calsepa (BA-8011-1, commercially available from EY Labs) was used as 5 μg/mL in carbo-free buffer supplemented with 2 mM CaCl₂) and MgCl₂. The biotinylated—ECL (B-1145-5, commercially available from Vector Laboratories) was used as 5 μg/mL in carbo-free buffer supplemented with 2 mM CaCl₂) and MgCl₂. The biotinylated—BanLec (B-1415, commercially available from Vector Laboratories) was used as 5 μg/mL in carbo-free buffer supplemented with 2 mM CaCl₂) and MgCl₂. After incubation, the slides were washed and Streptavidin—Cy3 (434315, commercially available from Thermo Fisher Scientific, and used as 2 μg/mL in carbo-free buffer supplemented with 2 mM CaCl₂) and MgCl₂) was added and incubated for 1 hour at room temperature. After incubation, the slides were washed and scanned by a scanner (InnoScan 710, commercially available from Innopsys) to obtain fluorescence signals, and the results are set forth in FIG. 13.

As demonstrated by FIG. 13, PNGase F treatment significantly reduced Calsepa, ECL and BanLec bindings to the S trimers. In that regard, Calsepa generally binds with all types of N-glycans. ECL recognizes terminal type 2 LacNAc epitope of N-glycans, and BanLec preferentially recognizes high-mannose and hybrid types of N-glycans. Therefore, this result verified that majority of N-glycans were removed from SARS-CoV-2 S trimers.

FIG. 13 also shows that PNGase F treatment also significantly reduced Calsepa binding to RBD. Unlike S trimers, there are no ECL and Banlec bindings observed for RBD. This finding suggests that the recombinant RBD lacks high-mannose and/or hybrid types of N-glycans or the high-mannose and/or hybrid types of N-glycans are terminated with sialic acids. In addition, N-glycans with terminal type 2 LacNAc epitope are also absent on RBD or these N-glycans are terminated with sialic acids. In that regard, Calsepa generally recognizes all types of N-glycans. Therefore, this result verified that majority of N-glycans were removed from RBD.

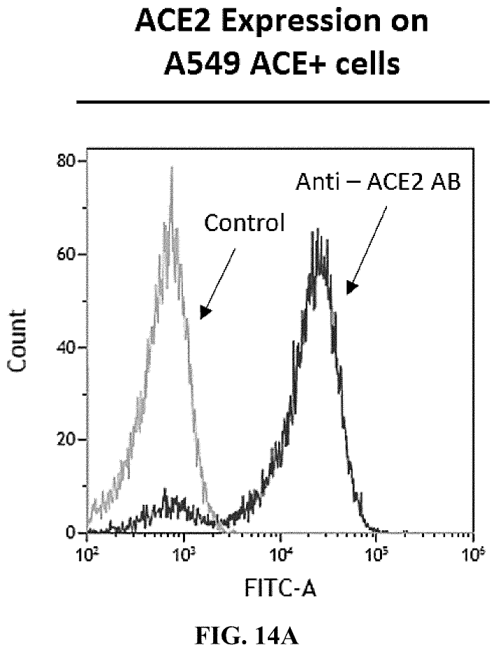

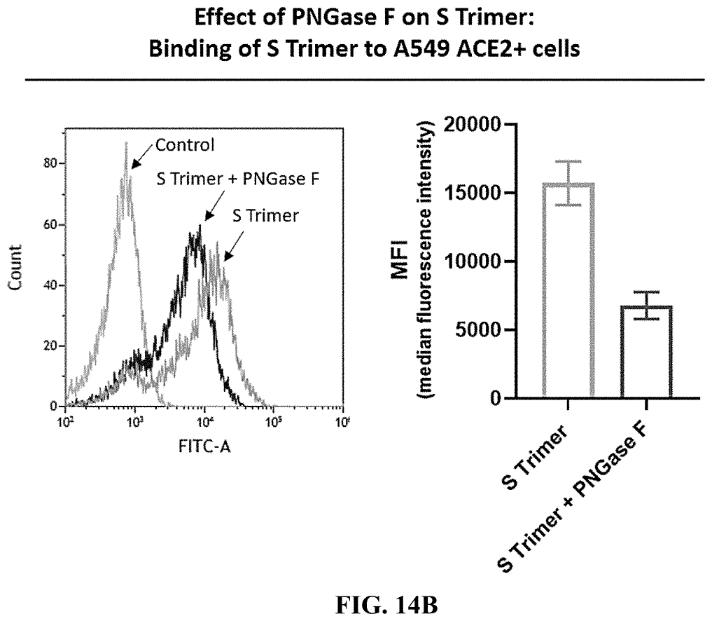

To validate these findings in live cells, cell bindings of native S trimers and S trimers processed with PNGase F were evaluated and compared. S trimers (SPN-C52H9, commercially available from Acro Biosystems, and used as 6 μg/100 μL) were mixed with vehicle (1×PBS) or vehicle containing PNGase F (P0704S, commercially available from NEB, and used as 1000 U/100 μL in 1×PBS). The same amount of native S Trimers and processed S Trimers were used to stain A549 ACE2+ cells for flow cytometry analysis. 3×105 A549 ACE2+ cells were incubated with the processed S Trimers or native S Trimers from vehicle control for 1 hour at room temperature. After incubation, the cells were washed and a mouse anti-S protein antibody (GTX632604, 1A9, commercially available from GeneTex, and used as 1:200) was used to stain the cells for 1 hour at 4° C., and after incubation, the cells were washed and a secondary goat anti-mouse IgG (H+L) antibody—FITC (F2761, commercially available from Invitrogen, and used as 8 μg/mL in 1×PBS) was used to stain the cells for 1 hour at 4° C. After incubation, the cells were washed and analyzed by flow cytometer (CytoFLEX, serial #BB51412, commercially available from Beckman Coulter), and the results are set forth in FIGS. 14A and 14B.

As demonstrated by FIGS. 14A and 14B, the PNGase F-processed S trimers present reduced binding to A549 ACE2+ cells as compared to the native S trimers. Together, these results indicate that decreasing the prevalence of N-glycans on SARS-CoV-2 S protein prevents SARS-CoV-2 S protein—ACE2 binding.

Data of flow cytometry was analyzed using CytExpert and Kaluza, both of which are commercially available from Beckman Coulter Diagnostics. Fluorescent signals of array slides were collected using Mapix (Innopsys). Data from the binding experiment was analyzed using GraphPad Prism V9.1.2 and an Analysis of Variance with Multiple Comparisons test.

### Example 5

This example demonstrates the ability to prevent SARS-CoV-2 Receptor Binding Domain (RBD)-ACE2 interaction, exhibited by the reduction of O-glycans on the RBD protein.

Recombinant SARS-CoV-2 S trimer and RBD proteins were immobilized on the multivalent N-hydroxysuccinimide (NHS) coated glass slide. The recombinant SARS-CoV-2 S protein (SPN-C52H9, commercially available from Acro Biosystems) was expressed in HEK293T cells and the recombinant RBD protein was expressed in the HEK293T cells. Recombinant S trimers and RBD (0.2 or 0.4 μg/μL) were immobilized on an NHS-functionalized slide by a microarray spotter, as described in Zhang (U.S. Patent Application Publication 2019/0128881), and a batch of array slides with immobilized S trimers and RBD were generated.

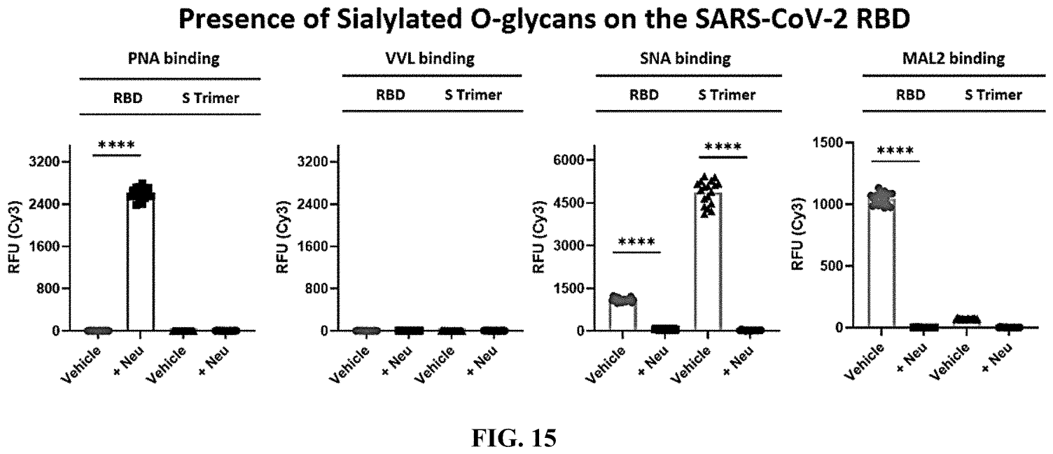

The slides were then treated with vehicle (1×PBS) or neuraminidase (11585886001—from Clostridium perfringens, commercially available from Roche, used as 50 mU/60 μL in 1×PBS per subarray) for 8 hours at room temperature. After treatment, the slides were washed and incubated with biotinylated Peanut Agglutinin (PNA) lectin (B-1075-5, commercially available from Vector Laboratories), biotinylated Vicia Villosa (VVL) lectin (B-1235-2, commercially available from Vector Laboratories), biotinylated Sambucus Nigra (SNA) lectin (B-1305-2, commercially available from Vector Laboratories) or biotinylated Maackia Amurensis Lectin II (MAL2) (B-1265-1, commercially available from Vector Laboratories). The biotinylated—PNA was used as 10 μg/mL in carbo-free buffer supplemented with 2 mM CaCl₂) and MgCl₂. The biotinylated—VVL was used as 10 μg/mL in carbo-free buffer supplemented with 2 mM CaCl₂) and MgCl₂. The biotinylated—SNA was used as 5 μg/mL in carbo-free buffer supplemented with 2 mM CaCl₂) and MgCl₂. The biotinylated-MAL2 was used as 5 μg/mL in carbo-free buffer supplemented with 2 mM CaCl₂) and MgCl₂. After incubation, the slides were washed and Streptavidin—Cy3 (434315, commercially available from Thermo Fisher Scientific, and used as 2 μg/mL in carbo-free buffer supplemented with 2 mM CaCl₂) and MgCl₂) was added and incubated for 1 hour at room temperature. After incubation, the slides were washed and scanned by a scanner (InnoScan 710, commercially available from Innopsys) to obtain fluorescence signals, and the results are set forth in FIG. 15.

As demonstrated by FIG. 15, neuraminidase treatment reduced SNA and MAL2 bindings to the RBD, while significantly enhanced PNA binding. In that regard, PNA recognizes T antigen and core 2 glycans without sialic acids and MAL2 and SNA binds to α2,3 and α2,6-linked sialic acids. Therefore, these results verified the presence of O-glycans on the recombinant RBD protein expressed in the HEK293 cells. Unlike PNA, VVL preferentially recognizes Tn antigen. Since there were no VVL bindings before and after neuraminidase treatment, Tn and STn antigen are not present on the RBD protein. Together, these results indicate that the O-glycans on the recombinant RBD protein are sialylated T and core 2 types of O-glycans. In contrast, neuraminidase treatment didn't enhance PNA and VVL bindings to the S trimers. Therefore, the O-glycans are absent from S trimers or they are not recognized by the lectins because of their hidden spatial conformations.

To validate whether O-glycans on the RBD influence ACE2 binding, recombinant RBD proteins were immobilized on the multivalent N-hydroxysuccinimide (NHS) coated glass slide, and the recombinant RBD protein was expressed in the HEK293T cells. Recombinant RBD protein (0.6 μg/μL) was immobilized on an NHS-functionalized slide by a microarray spotter, as described in U Zhang (U.S. Patent Application Publication 2019/0128881), and a batch of array slides with immobilized RBD were generated.

The slides were then treated with a panel of enzymes to deglycosylate the O-glycans on the RBD proteins. Neuraminidase (11585886001—from Clostridium perfringens, commercially available from Roche) was used as 50 mU/60 μL in 1×PBS per subarray; al-2,3,4,6 Fucosidase (P0748S, commercially available from New England Biolabs) was used as 8 U/60 μL in 1×PBS per subarray; β1-4 Galactosidase S (P0745S, commercially available from New England Biolabs) was used as 16 U/60 μL in 1×PBS per subarray; β-N-Acetylglucosaminidase S (P0744S, commercially available from New England Biolabs) was used as 8 U/60 μL in 1×PBS per subarray; and O-Glycosidase (P0733S, commercially available from New England Biolabs) was used as 200 mU/60 μL in 1×PBS per subarray.

Neuraminidase (Clostridium perfringens) cleaves terminal sialic acids that are α2,3-, α2,6-, or α2,8-linked to Gal, GlcNAc, GalNAc, AcNeu, GlcNeu, oligosaccharides, glycolipids, or glycoproteins. α1-2,3,4,6 Fucosidase hydrolyzes α1-2, α1-3, α1-4 and α1-6 linked fucose residues from oligosaccharides. β1-4 Galactosidase S hydrolyzes β1-4 linked galactose residues from oligosaccharides. β-N-Acetylglucosaminidase S hydrolyzes terminal, non-reducing β-N-Acetylglucosamine residues from oligosaccharides. O-Glycosidase removes Core 1 and Core 3 O-linked disaccharides from glycoproteins. In this regard, a combination of these enzymes was used to break down core 1, core 2, and their extended O-glycans on the SARS-CoV-2 RBD proteins.

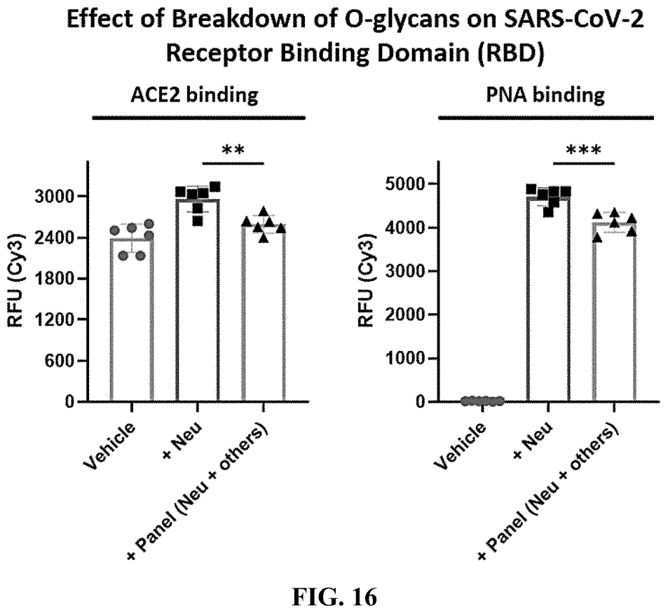

After treating the slides with the panel of enzymes for 8 hours at 37° C., the slides were washed, and incubated with recombinant human ACE2 (0108-H08H, commercially available from Sino Biological Inc., and used as 4 μg/mL in 1×PBS) for 1 hour at room temperature. After incubation, the slides were washed and a secondary rabbit anti-human ACE2 antibody (10108-RP01, commercially available from Sino Biological Inc., and used at a concentration of 2 μg/mL) was added and incubated for an additional 1 hour. After incubation, the slides were washed and a tertiary goat anti-rabbit IgG (H+L) Cy3 antibody (ab6838, commercially available from ABCAM, and used as 4 μg/mL) was added and incubated for 1 hour at room temperature. After incubation, the slides were washed and scanned by a scanner (InnoScan 710, commercially available from Innopsys) to obtain fluorescence signals, and the results are set forth in FIG. 16.

As demonstrated by the results set forth in FIG. 16, removal of sialic acids enhanced SARS-CoV-2 RBD-ACE2 binding. While sialic acids were removed, further enzymatic breakdown of O-glycans on the RBD protein moderately reduced RBD-ACE2 binding.

To validate whether O-glycans were removed from RBD, the enzyme—processed slides were incubated with biotinylated Peanut Agglutinin (PNA) lectin for 1 hour at room temperature. The biotinylated Peanut Agglutinin (PNA) lectin (B-1075-5, commercially available from Vector Laboratories) was used as 10 μg/mL in carbo-free buffer supplemented with 2 mM CaCl₂) and MgCl₂. After incubation, the slides were washed and Streptavidin—Cy3 (434315, commercially available from Thermo Fisher Scientific, and used as 2 μg/mL in carbo-free buffer supplemented with 2 mM CaCl₂) and MgCl₂) was added and incubated for 1 hour at room temperature. After incubation, the slides were washed and scanned by a scanner (InnoScan 710, commercially available from Innopsys) to obtain fluorescence signals, and the results are set forth in FIG. 16.

As demonstrated by FIG. 16, PNA binding was moderately reduced after enzyme treatment. In that regard, PNA preferentially recognizes T antigen, Core 2, and extended Core 2 O-glycan structures without terminal sialic acids. Therefore, this result verified that some O-glycans were removed from RBD protein.

### Example 6

This example demonstrates the ability to enhance the sialylation of cultured cells, exhibited by the treatment with D-mannose.

Representative cells from various tissue origins were treated with 100 mM D-mannose for 10 days and then stained by lectins for flow cytometry analysis. MCF7 (adenocarcinoma cells from mammary gland), PANC1 (epithelioid carcinoma cells from pancreas), and A549 (carcinoma cells from lung) cells were cultured in DMEM (Dulbecco's Modified Eagle's Medium with 4.5 g/L glucose, L-glutamine, and sodium pyruvate, 10% fetal bovine serum, 1% streptomycin, and penicillin) supplemented with or without 100 mM D-mannose (M8574, commercially available from Sigma) for 10 days. During the 10-day period of time, cells were split 3-4 times and 25% of the cell population was kept for continuous culturing under the same conditions. After 10 days, the D-mannose—treated and non-treated cells—were stained with biotinylated lectins. Lectin staining solutions were prepared by adding each lectin to the carbo-free buffer supplemented with 2 mM CaCl₂) and MgCl₂. Staining solution (100 μL) was used to stain 3.0×10⁵ cells for 1 hour at 4° C. MCF7 cells were stained with PNA (2 μg/mL), ECL (1.0 μg/mL), SNA (1.0 μg/mL), and MAL2 (0.8 μg/mL). PANC1 cells were stained with PNA (2.5 μg/mL), ECL (0.8 μg/mL), SNA (0.8 μg/mL), and MAL2 (0.5 μg/mL). A549 cells were stained with PNA (5 μg/mL), ECL (2 μg/mL), SNA (1 μg/mL), and MAL2 (6 μg/mL). Biotinylated-PNA (B-1075-5), ECL (B-1145-5), SNA (B-1305-2), and MAL2 (B-1265-1) were obtained from Vector Laboratories.

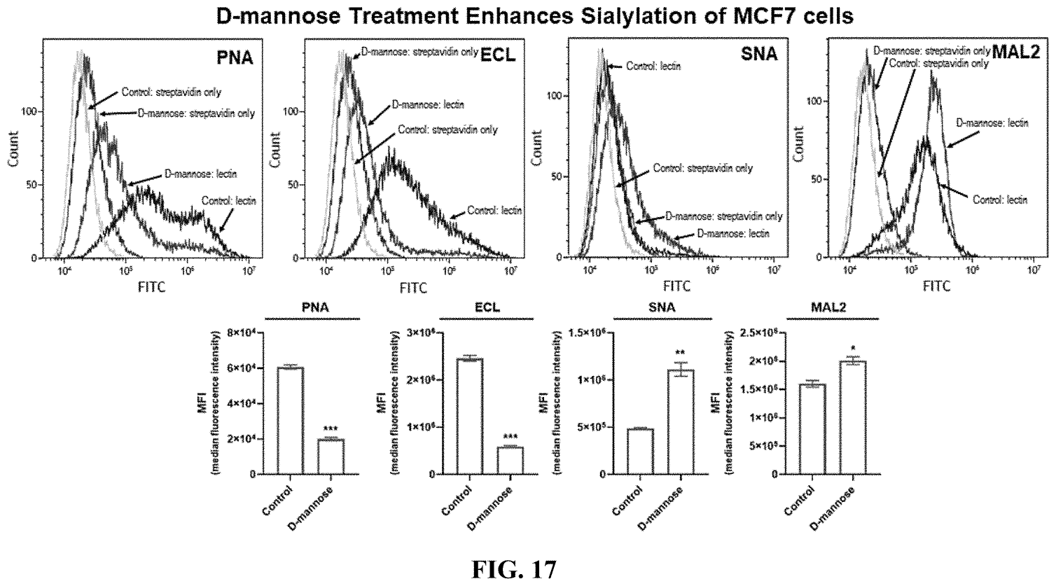

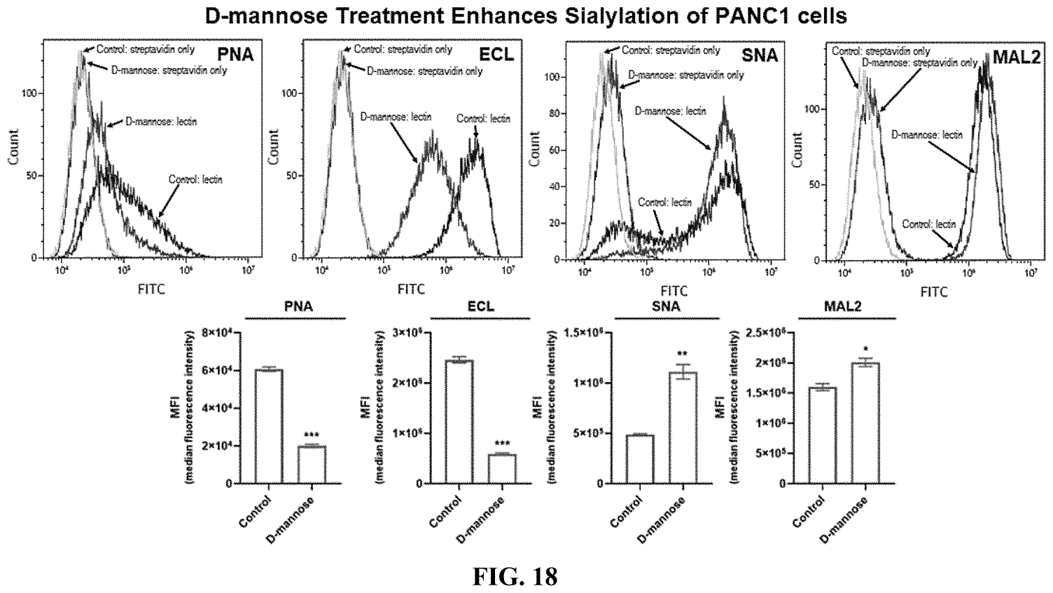

After incubation, the cells were washed and incubated with Streptavidin—FITC (10053373, commercially available from BD Pharmingen, and used as 10 μg/mL in carbo-free buffer supplemented with 2 mM CaCl₂) and MgCl₂) for 1 hour at 4° C. After incubation, the cells were washed and used for flow cytometry analysis, and the results are set forth in FIGS. 17-19.

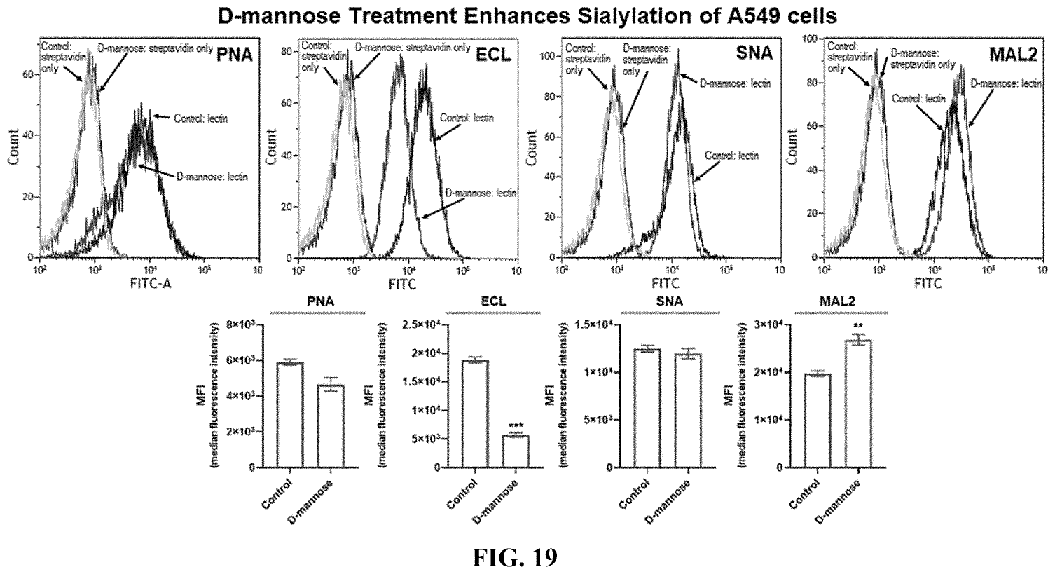

As demonstrated by FIGS. 17 and 18, for MCF7 cells and PANC1 cells, D-mannose treatment generally reduced PNA and ECL binding, while increasing SNA and MAL2 binding. This result indicates that D-mannose treatment enhanced the presence of α(2,3)- and α(2,6)-linked sialic acids on the cell membrane glycoconjugates. As demonstrated by FIG. 19, for A549 cells, D-mannose treatment significantly reduced ECL binding, while slightly enhancing MAL2 binding. D-mannose treatment had minimal to no influence on PNA and SNA binding. This result indicates that sialic acids were mostly added to the N-glycans by the α(2,3) linkage and possibly with polysialic acids. Together, these results indicate that D-mannose treatment enhanced sialylation process of cultured cells.

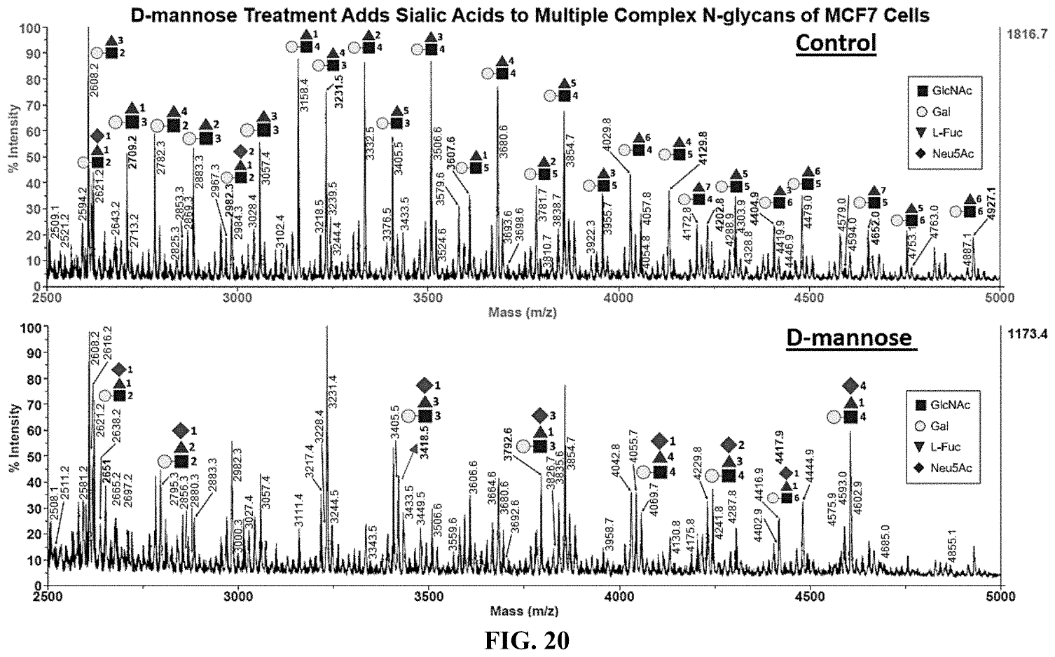

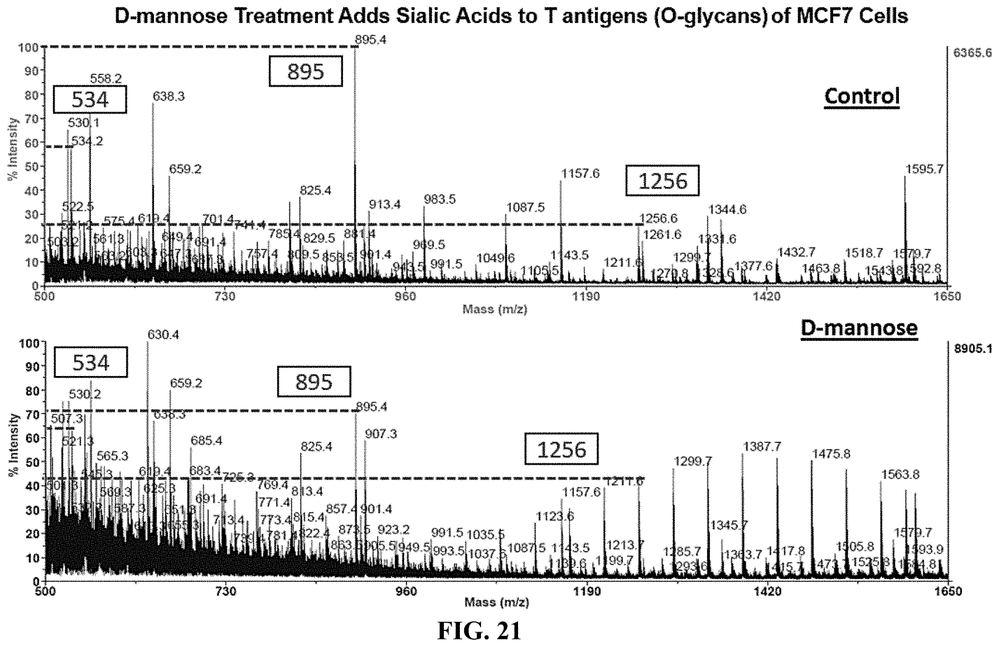

To further validate these results, the cell surface O- or N-glycans from the D-mannose—treated or non-treated cells—were analyzed by mass spectrometry. MCF7 and PNAC1 cells were evaluated. For each sample, an equal number of cells (4.0×10⁶) were washed in 1×PBS for three times. The resulting cell pellets were then washed with PBS buffer and were dissolved in a lysis buffer (1% Triton X-100 in TBS) and cells were lysed using sonication. After sonication, the samples were centrifuged, and the supernatant fractions were reduced using DTT and alkylated using iodoacetamide. The samples were then dialyzed against water, which was changed every 4-6 hours, at 4° C. for 48 hours to remove residual urea. The sample solutions were then concentrated without drying out the samples and again reconstituted in 50 mM ammonium bicarbonate. The N-glycans were released from the proteins by adding N-glycosidase F (PNGase F) at 37° C. for 12 hours. The released N-glycans were filtered through a 10 KDa cutoff filter. O-glycoproteins from the top of the filter were subjected to β-elimination. For β-elimination, the O-glycoproteins were treated with a mixture of 50 mM NaOH solution and sodium borohydride (NaBH₄) solution in 50 mM NaOH solution. The resulting samples were heated to 45° C. for 18 hours, cooled, neutralized by 10% acetic acid, passed through a Dowex H+ resin column and C18 column, lyophilized, and the borates were removed under the stream of nitrogen using methanol and acetic acid mixture. The released O-linked oligosaccharides were permethylated by using methyl iodide in a DMSO/NaOH mixture. The reaction was quenched with water and the reaction mixture was extracted with methylene chloride and dried. The dried glycans were re-dissolved in methanol and profiled by MALDI-TOF. The results for the N-glycans of MCF7 cells are set forth in FIG. 20 and the results for the O-glycans of MCF7 and PNAC1 cells are set forth in FIGS. 21-23, respectively.

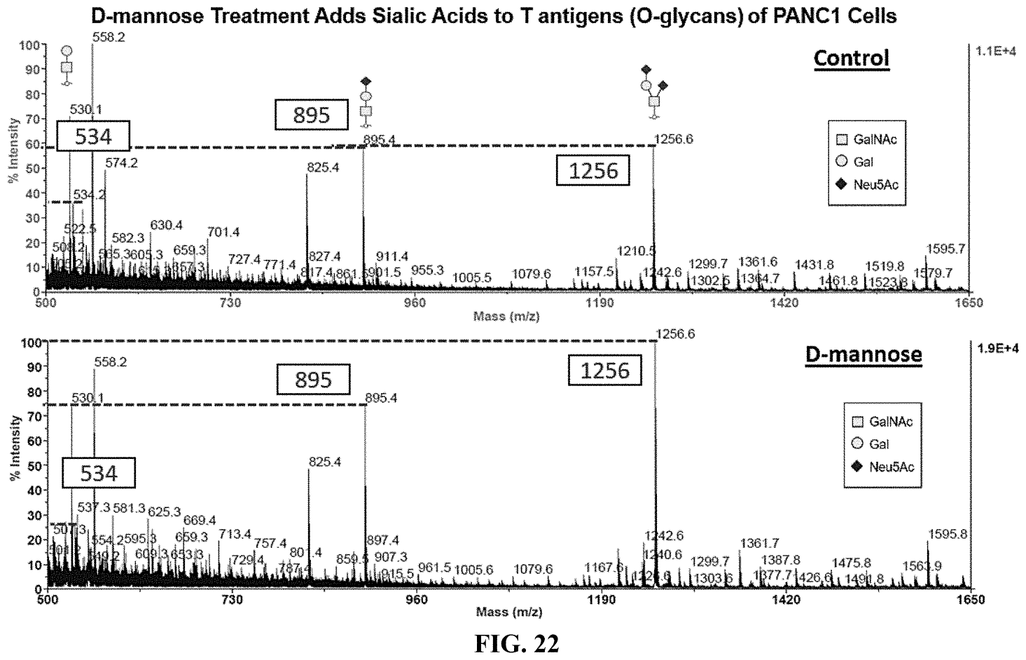

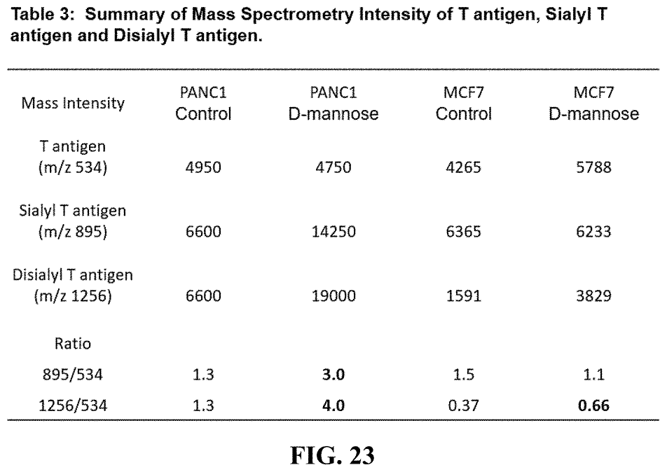

As demonstrated by FIG. 20, for MCF7 cells, sialic acids were added to multiple N-glycans. As demonstrated by FIGS. 21 and 23, for O-glycans, D-mannose treatment significantly enhanced the disialyl T antigen/T antigen ratio, indicating that more sialic acids were added to the T antigens. As demonstrated by FIGS. 22 and 23, for PANC1 cells, both the sialyl T antigen/T antigen and the disialyl T antigen/T antigen ratios were significantly increased, indicating the enhanced presence of sialic acids on T antigens. Together, these results indicate that D-mannose treatment enhanced the cellular sialylation process and increased presence of sialic acids on various glycans.

Data of flow cytometry was analyzed using Kaluza, commercially available from Beckman Coulter Diagnostics, GraphPad Prism V9.1.2, and an Analysis of Variance with Multiple Comparisons test.

### Example 7

This example demonstrates the ability to reduce N-glycosylation and/or O-glycosylation of cultured cells, exhibited by the treatment with D-mannose or a derivative of D-mannose.

Representative cells from various tissue origins were treated with 100 mM D-mannose for 10 days and then stained by lectins for flow cytometry analysis. LN18 (glioblastoma cells from brain) and MDA-MB-231 (adenocarcinoma cells from mammary gland) cells were cultured in DMEM (Dulbecco's Modified Eagle's Medium with 4.5 g/L glucose, L-glutamine, and sodium pyruvate, 10% fetal bovine serum, 1% streptomycin, and penicillin) supplemented with or without 100 mM D-mannose (M8574, commercially available from Sigma) for 10 days. During the 10-day period of time, cells were split 3-4 times and 25% of the cell population was kept for continuous culturing under the same conditions. After 10 days, the D-mannose—treated and non-treated cells—were stained with biotinylated lectins. Lectin staining solutions were prepared by adding each lectin to the carbo-free buffer supplemented with 2 mM CaCl₂) and MgCl₂. Staining solution (100 μL) was used to stain 3.0×10⁵ cells for 1 hour at 4° C. LN18 cells were stained with PNA (1.8 μg/mL), ECL (1.4 μg/mL), SNA (1.6 μg/mL), and MAL2 (0.8 μg/mL). MDA-MB-231 cells were stained with PNA (1.5 μg/mL), ECL (1.5 μg/mL), SNA (0.8 μg/mL), and MAL2 (0.8 μg/mL). Biotinylated—PNA (B-1075-5), ECL (B-1145-5), SNA (B-1305-2), and MAL2 (B-1265-1) were obtained from Vector Laboratories.

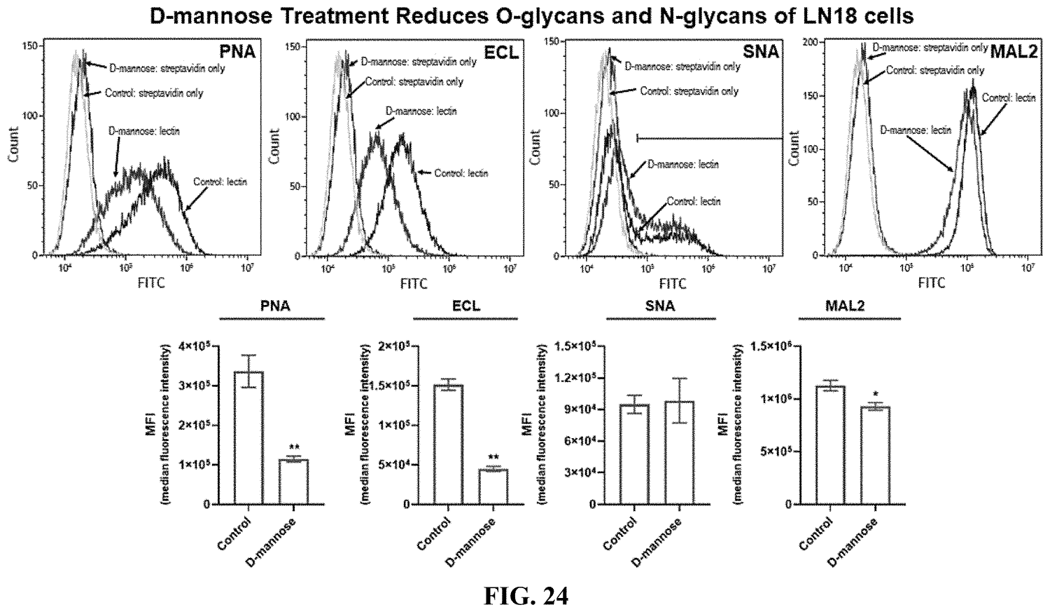

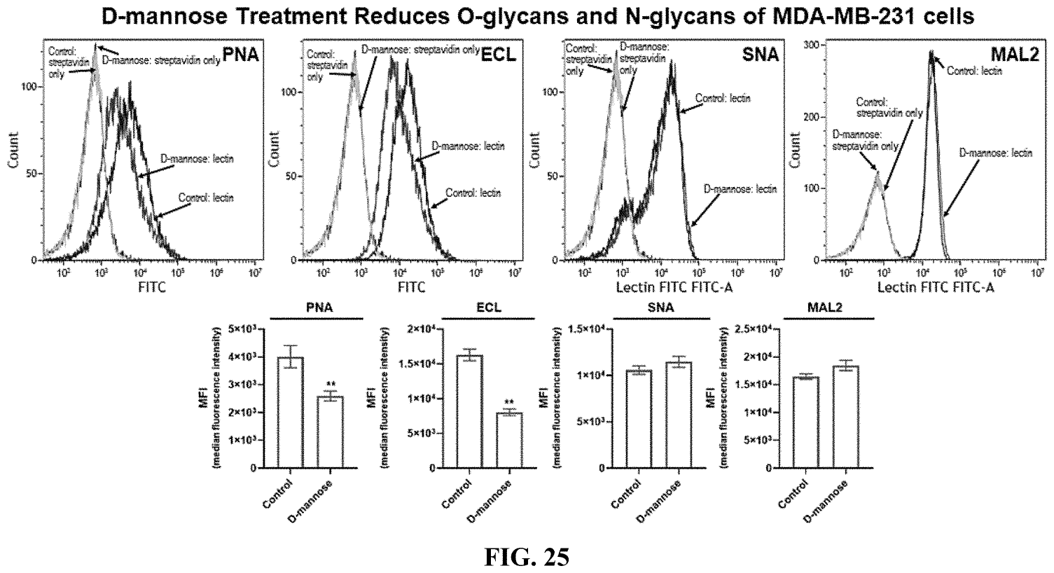

After incubation, the cells were washed and incubated with Streptavidin—FITC (10053373, commercially available from BD Pharmingen, and used as 10 μg/mL in carbo-free buffer supplemented with 2 mM CaCl₂) and MgCl₂) for 1 hour at 4° C. After incubation, the cells were washed and used for flow cytometry analysis, and the results are set forth in FIGS. 24 and 25.

As demonstrated by FIGS. 24 and 25, for LN18 cells and MDA-MB-231 cells, D-mannose treatment significantly reduced PNA and ECL bindings, while having no effect on enhancing SNA and MAL2 bindings. This result indicates that D-mannose treatment attenuated glycosylation process of these cells and reduced the presence of N-glycans and O-glycans on the cell surface.

To investigate whether 1,4-di-O-acetyl-D-mannopyranose reduces N- and O-glycosylation of cultured cells, A375 (malignant melanoma cells from skin) cells were treated with 5 mM 1,4-di-O-acetyl-D-mannopyranose for 3 days and then stained by lectins for flow cytometry analysis. A375 cells were cultured in DMEM (Dulbecco's Modified Eagle's Medium with 4.5 g/L glucose, L-glutamine, and sodium pyruvate, 10% fetal bovine serum, 1% streptomycin, and penicillin) supplemented with or without 5 mM 1,4-di-O-acetyl-D-mannopyranose for 3 days. After 3 days, the 1,4-di-O-acetyl-D-mannopyranose—treated and non-treated cells—were stained with biotinylated lectins. Lectin staining solutions were prepared by adding each lectin to the carbo-free buffer supplemented with 2 mM CaCl₂) and MgCl₂. Staining solution (100 μL) was used to stain 3.0×10⁵ cells for 1 hour at 4° C. A375 cells were stained with PNA (10 μg/mL), ECL (15 μg/mL), SNA (6 μg/mL), and MAL2 (3 μg/mL). Biotinylated—PNA (B-1075-5), ECL (B-1145-5), SNA (B-1305-2), and MAL2 (B-1265-1) were obtained from Vector Laboratories.

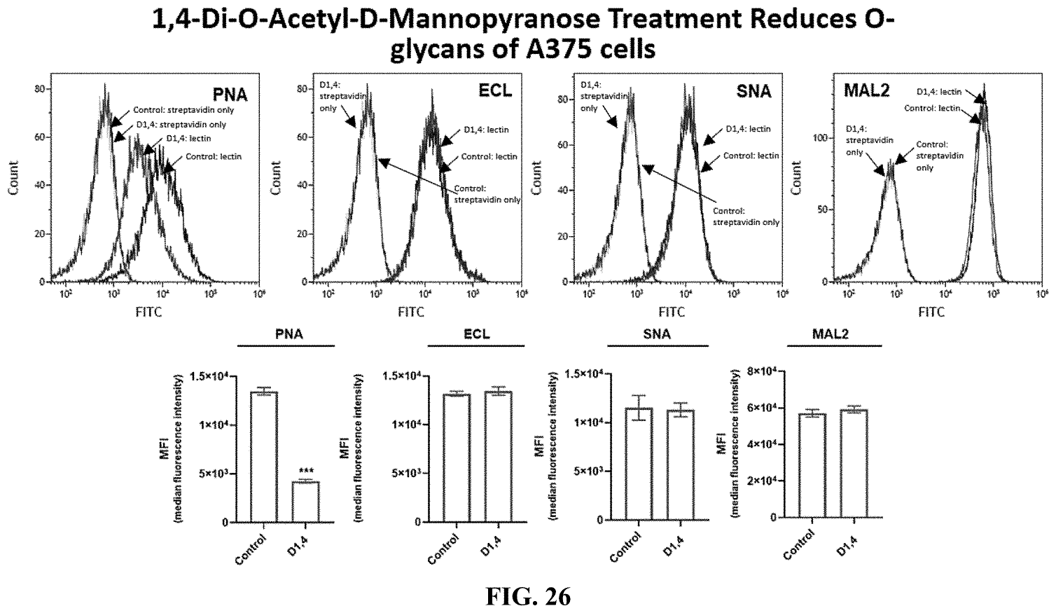

After incubation, the cells were washed and incubated with Streptavidin—FITC (10053373, commercially available from BD Pharmingen, and used as 10 μg/mL in carbo-free buffer supplemented with 2 mM CaCl₂) and MgCl₂) for 1 hour at 4° C. After incubation, the cells were washed and used for flow cytometry analysis, and the results are set forth in FIG. 26.

As demonstrated by FIG. 26, for A375 cells, 1,4-di-O-acetyl-D-mannopyranose treatment significantly reduced PNA bindings, while having no effect on ECL, SNA and MAL2 bindings. This result indicates that 1,4-di-O-acetyl-D-mannopyranose treatment attenuated O-glycosylation process of A375 cells and reduced the presence of O-glycans on the cell surface.

To investigate whether 1,2,3,4-tetra-O-Acetyl-D-mannopyranose reduces N- and O-glycosylation of cultured cells, MCF7 (adenocarcinoma cells from mammary gland) cells were treated with 1 mM 1,2,3,4-tetra-O-Acetyl-D-mannopyranose for 10 days and then stained by lectins for flow cytometry analysis. MCF7 cells were cultured in DMEM (Dulbecco's Modified Eagle's Medium with 4.5 g/L glucose, L-glutamine, and sodium pyruvate, 10% fetal bovine serum, 1% streptomycin, and penicillin) supplemented with or without 1 mM 1,2,3,4-tetra-O-Acetyl-D-mannopyranose for 10 days. During the 10-day period of time, cells were split 2 times and 50% of the cell population was kept for continuous culturing under the same conditions. After 10 days, the 1,2,3,4-tetra-O-Acetyl-D-mannopyranose—treated and non-treated cells—were stained with biotinylated lectins. Lectin staining solutions were prepared by adding each lectin to the carbo-free buffer supplemented with 2 mM CaCl₂) and MgCl₂. Staining solution (100 μL) was used to stain 3.0×10⁵ cells for 1 hour at 4° C. MCF7 cells were stained with PNA (5 μg/mL), ECL (5 μg/mL), SNA (1.0 μg/mL), and MAL2 (10 μg/mL). Biotinylated—PNA (B-1075-5), ECL (B-1145-5), SNA (B-1305-2), and MAL2 (B-1265-1) were obtained from Vector Laboratories.

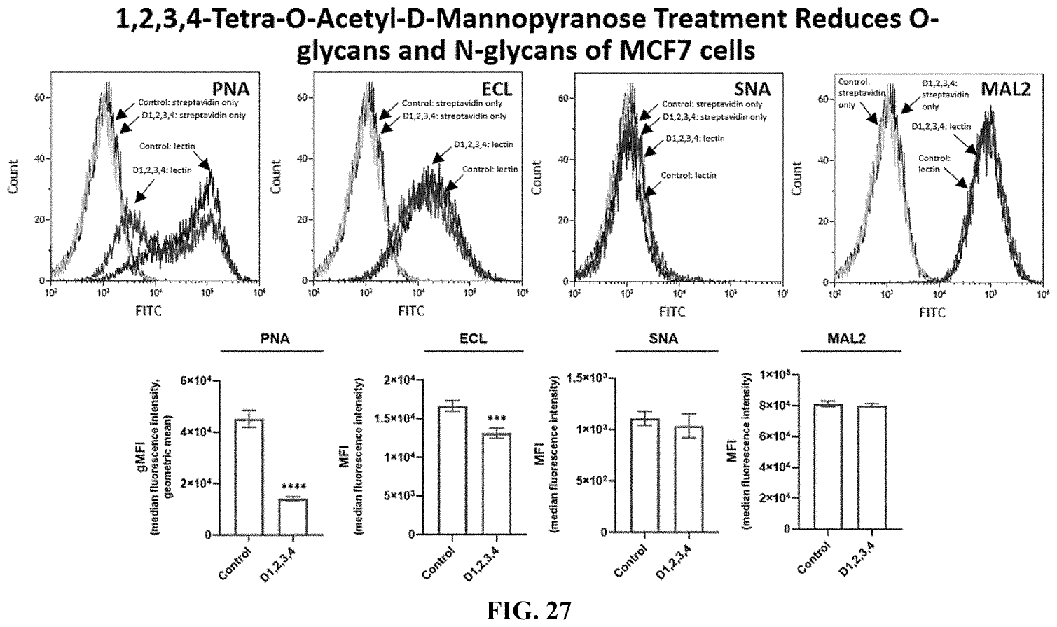

After incubation, the cells were washed and incubated with Streptavidin—FITC (10053373, commercially available from BD Pharmingen, and used as 10 μg/mL in carbo-free buffer supplemented with 2 mM CaCl₂) and MgCl₂) for 1 hour at 4° C. After incubation, the cells were washed and used for flow cytometry analysis, and the results are set forth in FIG. 27.

As demonstrated by FIG. 27, for MCF7 cells, 1,2,3,4-tetra-O-Acetyl-D-mannopyranose treatment significantly reduced PNA bindings and moderately reduced ECL binding, while having no effect on SNA and MAL2 bindings. This result indicates that 1,2,3,4-tetra-O-Acetyl-D-mannopyranose treatment attenuated O- and N-glycosylation process of MCF7 cells and reduced the presence of O-glycans and N-glycans on the cell surface.

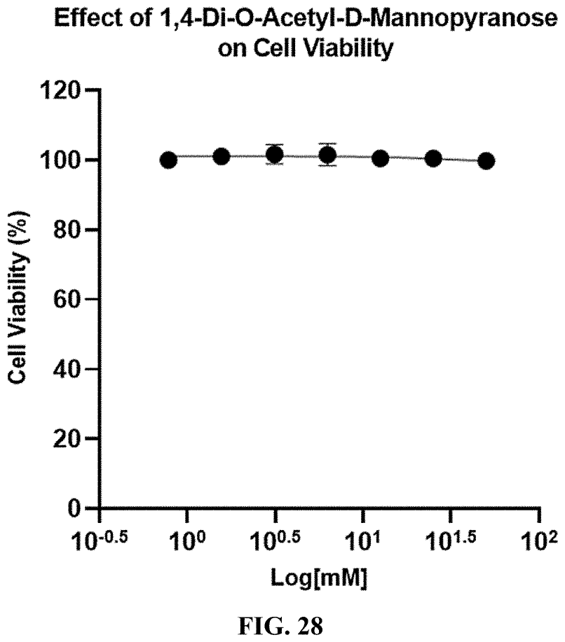

To determine the cytotoxic effect of 1,4-di-O-acetyl-D-mannopyranose, a cell viability assay was performed. A375 cells were seeded into 96-well cell culture plate as a density of 6500 cells/well. After leaving the cells overnight, the cells were treated with vehicle or 50 mM, 25 mM, 12.5 mM, 6.25 mM, 3.1 mM, 1.56 mM and 0.78 mM of 1,4-di-O-acetyl-D-mannopyranose. After 72 hours, cell viability was evaluated by Cell Counting Kit-8 (CK04, commercially available from Dojindo Molecular Technologies), and the results are set forth in FIG. 28.

As demonstrated by FIG. 28, treatment with 1,4-di-O-acetyl-D-mannopyranose did not have any influence on cell viability. This result indicates that 1,4-di-O-acetyl-D-mannopyranose does not have a cytotoxic effect on the cultured cells.

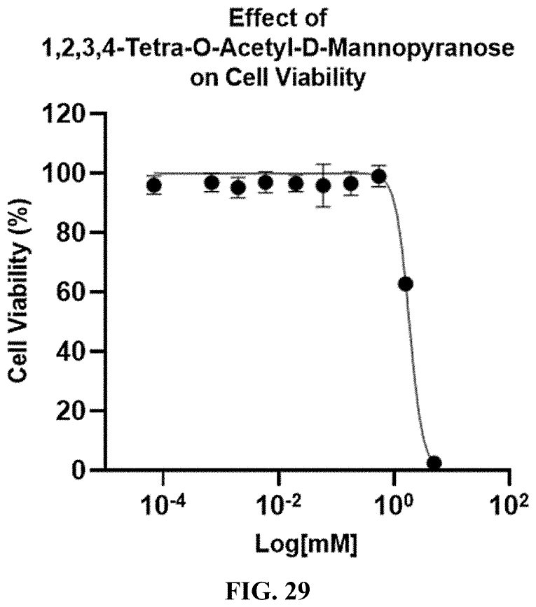

To determine the cytotoxic effect of 1,2,3,4-tetra-O-Acetyl-D-mannopyranose, a cell viability assay was performed. MCF7 cells were seeded into 96-well cell culture plate as a density of 10000 cells/well. After leaving the cells overnight, the cells were treated with vehicle or 5 mM, 1.6 mM, 0.55 mM, 0.18 mM, 0.06 mM, 0.02 mM, 0.006 mM, 0.002 mM, 0.0007 mM, and 0.00007 mM of 1,2,3,4-tetra-O-Acetyl-D-mannopyranose. After 72 hours, cell viability was evaluated by Cell Counting Kit-8 (CK04, commercially available from Dojindo Molecular Technologies), and the results are set forth in FIG. 29.

As demonstrated by FIG. 29, treatment with 1,2,3,4-tetra-O-Acetyl-D-mannopyranose induced cell toxicity at a higher concentration while having no impact on cell viability at concentrations less than 0.7 mM.

### Example 8

This example demonstrates the ability to reduce N-glycosylation of SARS-CoV-2 S1 protein expressed in the cultured cells, exhibited by the treatment with D-mannose.

SARS-CoV-2 is fully reliant on the protein synthesis machinery of host cells and subverts host glycosylation machinery to express and glycosylate its own viral glycoproteins. To investigate whether D-mannose influences glycosylation of SARS-CoV-2 S protein expressed in the cultured cells, a plasmid was generated to express SARS-CoV-2 S1 protein under D-mannose treatment. The plasmid was designed for expressing codon-optimized SARS-CoV-2 S1 protein with a C-terminal human Fc-tag. This plasmid encodes S1 protein from the original isolate first identified in Wuhan, and contains a SV40 enhancer, a human EF1α-HTLV composite promoter and an exogenous signal sequence to maximize protein secretion and production.

Representative host cells were treated with 100 mM D-mannose for 7 days and then transfected with SARS-CoV-2 S1 expression plasmid. HEK293F cells were cultured in DMEM (Dulbecco's Modified Eagle's Medium with 4.5 g/L glucose, L-glutamine, and sodium pyruvate, 10% fetal bovine serum, 1% streptomycin, and penicillin) supplemented with or without 100 mM D-mannose (M8574, commercially available from Sigma) for 7 days. During the 7-day period of time, cells were split 2-3 times and 25% of the cell population was kept for continuous culturing under the same conditions. After 7 days, the D-mannose—treated and non-treated cells were transfected with SARS-CoV-2 S1 expression plasmid by using PureFection transfection reagent (LV750A, commercially available from System Biosciences). After sitting overnight, chemically defined and protein-free culture medium (12338018, commercially available from Thermo Fisher Scientific) was replaced to collect SARS-CoV-2 S1 protein expressing in the host cells. For D-mannose treated cells, D-mannose was supplemented during the entire process of cell transfection and protein expression. At the end of the experiment, expression of S1 proteins were validated and the concentrations of the proteins were determined.

To capture the FC-fusion SARS-CoV-2 S1 proteins on microarray slides for functional glycosylation analysis, goat anti-human IgG Fc antibody (ab97221, commercially available from abcam) was immobilized on the epoxysilane coated microarray slides (commercially available from SCHOTT). The Goat anti-human IgG Fc antibody (0.5 μg/μL) was immobilized on the epoxy coated slide by a microarray spotter, as described in Zhang (U.S. Patent Application Publication 2019/0128881), and a batch of capture microarray slides were generated. Vehicle control and SARS-CoV-2 S1 proteins (70 μg/mL) were incubated with the capture microarray overnight at room temperature.

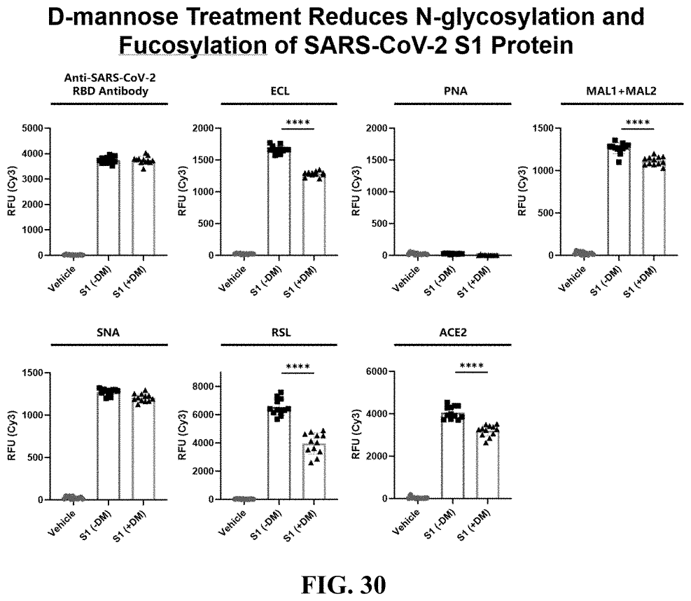

To validate the presence of S1 protein on the capture microarray, the slides were washed and incubated with mouse anti-RBD antibody (MAB10540, commercially available from R&D) for 1 hour at room temperature. The mouse anti-RBD antibody was used as 2 μg/mL in TBS-T buffer. After incubation, the slides were washed and goat anti-mouse IgG (AF555) (A21422, commercially available from Thermo Fisher Scientific, and used as 5 μg/mL in TBS-T buffer) was added and incubated for 1 hour at room temperature. After incubation, the slides were washed and scanned by a scanner (InnoScan 710, commercially available from Innopsys) to obtain fluorescence signals, and the results are set forth in FIG. 30.

As demonstrated by FIG. 30, nearly equal amount of S1 protein were captured by the immobilized anti-human IgG Fc antibody. As expected, there is no presence of S1 protein in the vehicle control.

To analyze glycosylation of captured S1 proteins, S1 protein—captured slides were incubated with biotinylated—Erythrina Cristagalli lectin (ECL), Peanut Agglutinin (PNA) lectin, Maackia Amurensis Lectin I (MAL1) and Maackia Amurensis Lectin II (MAL2), Sambucus Nigra (SNA) lectin, or Ralstonia solanacearum lectin (RSL) for 1 hour at room temperature. The biotinylated—ECL (B-1145-5, commercially available from Vector Laboratories) was used as 5 μg/mL in carbo-free buffer supplemented with 2 mM CaCl₂) and MgCl₂. The biotinylated—PNA (B-1075-5, commercially available from Vector Laboratories) was used as 10 μg/mL in carbo-free buffer supplemented with 2 mM CaCl₂) and MgCl₂. The biotinylated—MAL1 and MAL2 (B-1315-2 and B-1265-1, commercially available from Vector Laboratories) were used as 10 μg/mL for each lectin in carbo-free buffer supplemented with 2 mM CaCl₂) and MgCl₂. The biotinylated—SNA (BA-6802-1, commercially available from EY Laboratories) was used as 10 μg/mL in carbo-free buffer supplemented with 2 mM CaCl₂) and MgCl₂. The biotinylated—RSL (L1258, commercially available from GLYcoDiag) was used as 4 μg/mL in carbo-free buffer supplemented with 2 mM CaCl₂) and MgCl₂. After incubation, the slide was washed and Streptavidin—Cy3 (434315, commercially available from Thermo Fisher Scientific, and used as 2 μg/mL in carbo-free buffer supplemented with 2 mM CaCl₂) and MgCl₂) was added and incubated for 1 hour at room temperature. After incubation, the slide was washed and scanned by a scanner (InnoScan 710, commercially available from Innopsys) to obtain fluorescence signals. The results are set forth in FIG. 30.

As demonstrated by FIG. 30, ECL and RSL bindings were significantly reduced for the S1 protein expressed in HEK293F cells under D-mannose treatment. Although there was no change for SNA binding, MAL1 and MAL2 bindings were moderately reduced. No PNA binding was observed. In that regard, ECL recognizes terminal type 2 LacNAc epitope of an N-glycan and RSL recognizes all fucose-containing N-glycans. Therefore, this result verified that D-mannose altered glycan biosynthesis of host cells to reduce the presence of N-glycans and fucose-containing N-glycans on the SARS-CoV-2 S1 protein.

To evaluate ACE2 binding, S1 protein—captured slides were incubated with recombinant human ACE2 (0108-H08H, commercially available from Sino Biological Inc., and used as 10 μg/mL in TBS-T buffer) for 1 hour at room temperature. After incubation, the slide was washed and a secondary rabbit anti-human ACE2 antibody (10108-RP01, commercially available from Sino Biological Inc., and used at a concentration of 4 μg/mL) was added and incubated for an additional 1 hour. After incubation, the slide was washed and a tertiary goat anti-rabbit IgG (H+L) Cy3 antibody (ab6838, commercially available from ABCAM, and used as 4 μg/mL) was added and incubated for 1 hour at room temperature. After incubation, the slide was washed and scanned by a scanner (InnoScan 710, commercially available from Innopsys) to obtain fluorescence signals. The results are set forth in FIG. 30.

As demonstrated by the results set forth in FIG. 30, reduced ACE2 binding was observed for the S1 protein expressed in the HEK293F cells under D-mannose treatment. Together, these results show biological activity of D-mannose in modulating glycosylation of SARS-CoV-2 viral glycoproteins.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of ameliorating and/or preventing a coronavirus infection in a subject, the method comprising administering to the subject a therapeutically effective amount of a sugar or a derivative thereof selected from D-mannose, fructose, neuraminic acid, mannosamine, glucosamine, galactosamine, a metabolite thereof, a prodrug thereof, or a combination thereof.

2. The method of claim 1, wherein the sugar or a derivative thereof is D-mannose, mannose-6-phosphate (Man-6-P), fructose-6-phosphate (Fruc-6-P), uridine 5′-diphospho-N-acetylglucosamine (UDP-GlcNAc), mannose-1-phosphate (Man-1-P), guanosine diphosphate mannose (GDP-Man), N-acetylglucosamine (GlcNAc), N-acetylgalactosamine (GalNAc), uridine 5′-diphospho-N-acetylgalactosamine (UDP-GalNAc), N-acetyl-D-mannosamine (ManNAc), N-acetyl-D-mannosamine-6-phosphate (ManNAc-6-P), N-acetylneuraminic-acid-9-phosphate (Neu5Ac-9-P), N-acetylneuraminic acid (Neu5Ac), cytidine-5′-monophospho-N-acetylneuraminic acid (CMP-Neu5Ac), acylated D-mannose, a metabolite thereof, a prodrug thereof, or a combination thereof.

3. The method of claim 1, wherein the coronavirus infection is a severe acute respiratory syndrome coronavirus 2 (SARS-CoV-2) related infection.

4. The method of claim 3, wherein the severe acute respiratory syndrome coronavirus 2 (SARS-CoV-2) related infection is COVID-19.

5. The method of claim 1, wherein the coronavirus infection is a severe acute respiratory syndrome coronavirus (SARS-CoV) related infection.

6. The method of claim 1, wherein the coronavirus infection is a Middle East respiratory syndrome (MERS) related infection.

7. The method of claim 1, wherein the sugar or a derivative thereof is administered orally, rectally, transmucosally, intestinally, parenterally, intramuscularly, subcutaneously, intradermally, intramedullaryly, intrathecally, intraventricularly, intravenously, intraperitoneally, intranasally, intraocularly, inhalationally, insufflationally, topically, cutaneously, transdermally, intra-arterially, or a combination thereof.

8. The method of claim 1, wherein the sugar or a derivative thereof is administered orally.

9. The method of claim 1, wherein the sugar or a derivative thereof is administered intravenously.

10. The method of claim 1, wherein the sugar or a derivative thereof is D-mannose.

11. The method of claim 1, wherein the sugar or a derivative thereof is mannose-6-phosphate (Man-6-P), fructose-6-phosphate (Fruc-6-P), uridine 5′-diphospho-N-acetylglucosamine (UDP-GlcNAc), mannose-1-phosphate (Man-1-P), guanosine diphosphate mannose (GDP-Man), N-acetylglucosamine (GlcNAc), N-acetylgalactosamine (GalNAc), uridine 5′-diphospho-N-acetylgalactosamine (UDP-GalNAc), N-acetyl-D-mannosamine (ManNAc), N-acetyl-D-mannosamine-6-phosphate (ManNAc-6-P), N-acetylneuraminic-acid-9-phosphate (Neu5Ac-9-P), N-acetylneuraminic acid (Neu5Ac), cytidine-5′-monophospho-N-acetylneuraminic acid (CMP-Neu5Ac), or a combination thereof.

12. The method of claim 1, wherein the sugar or a derivative thereof is an acylated D-mannose is of the formula: 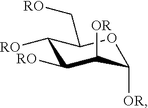
wherein each R independently is hydrogen or 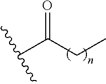
and n is an integer from 0 to 17, and at least one R is

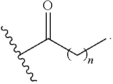

13. The method of claim 9, wherein administering the therapeutically effective amount of the sugar or a derivative thereof achieves a plasma concentration of at least 2 times greater than a plasma concentration of said sugar or said derivative thereof prior to administration.

14. The method of claim 1, wherein administering the therapeutically effective amount of the sugar or a derivative thereof upregulates sialylation of a glycan of a virus particle of the coronavirus.

15. The method of claim 14, wherein sialylation of the virus particle of the coronavirus is upregulated by at least 4% relative to a virus particle of the coronavirus in the subject prior to being treated with the sugar or the derivative thereof.

16. The method of claim 1, wherein administering the therapeutically effective amount of the sugar or a derivative thereof downregulates N-glycosylation of a virus particle of the coronavirus.

17. The method of claim 16, wherein N-glycosylation of the virus particle of the coronavirus is downregulated by at least 4% relative to a virus particle of the coronavirus in the subject prior to being treated with the sugar or the derivative thereof.

18. The method of claim 1, wherein administering the therapeutically effective amount of the sugar or a derivative thereof downregulates O-glycosylation of a virus particle of the coronavirus.

19. The method of claim 18, wherein O-glycosylation of the virus particle of the coronavirus is downregulated by at least 4% relative to a virus particle of the coronavirus in the subject prior to being treated with the sugar or the derivative thereof.

20. A method of ameliorating and/or preventing a coronavirus infection in a subject, the method comprising upregulating sialylation of a glycan, downregulating N-glycosylation, and/or downregulating O-glycosylation of a virus particle of the coronavirus.
